SEQUENCE LISTING (6 sequences)

SEQ ID NO: 1 - DNA, 21004 bp, Shewanella baltica
cagagcaccactatgccttcaagccttcgccttgtctaaagcgcgttgaactcccgctgaatgaacagatatttaatacgattggtattaaactggctgataacaagataacgcgcagaaagaattatggctaaacgcaatcgcagatcttagcggtttaggtgagtttactggcgcgaagaaagcactctctttcgccgttaataatcagcaagtaagctctctatgctgctcaactgcgcggttagcgttatgggaagcgcggcattaccggcatgcgctaagctaatggcaaagcgatactcctcggtcaaacgaccaaaatgcgtttgccaatgcaaggagtcagctaaatccagtgtgtgtattgggatttgtagcgggacttgtaatggaatatgtggataaacattaagcattgaagctacgccgcaggcttgcctcaatccggcatcttggcatgataactcaagcgattctgccgccaattgctcaaacggcttaagctcaaaggcaaaggatttgagcgactgcgctaaacctaaaccagtcgccttaatataggactcctttagcgcccataaatcgaaaaagcgctgtcgctgactcgcttcatcggcgagagcgagcaaggctgcagtttcttggggagaaaaatagtgatttaggattggatagatatccgtcttcggccgcgacctttcaatatccaccccaaataaaccagactcaacgccatcaaactgagccacaccaatcaataaccaatcgccgctatggcttaggttaaactcgagaccagtttgctgccctaatgtcgccgttaaactcggtttgcccttagcgccatattcaaaacaccactcatgtggcgataaatcagcatagcgagataacacggtccgtagcgccgccctcacctgtaacccctttatttgcaccttgggatcgcgataacgcctgacctttgcgagttcatcatcacttaaccaagactcggcaagtgatgtttgcgccgctgaaatatcgttcagtggaataaaaaatagatccacattcacgccgtttttgctagatgttagagagtcggtcattcgagagcctagctgtacaaagccctgcggctgatgagttatcattggctggctttttggagagcagttagcaacattcttatctggttcattattgtctaggttcatcattatctgagttcattatgcatcttaaatagttccaactaagccctatttcaattcttagccgcactctaataagagcaattcttagaacgactattaactctatcctaagcgcaaaattttgctgcaattgcgccaaggttcccccccaaaaaaaattagcaggattagccacacagcctcattcatgactaaagggacgcaagccaatattgggctattttaccccgataactcaaaaagctgccttcatcgccgctacaaatataaatcttccactatcaaagtattaaacagcccaagcggccagcgaaaacccaacataacgcacagaaaataccattactcaacgaaagccaataaattccagtcccagatgattgtatcgacccgaaacctcaggtaatctgcatccccattaagtgacacagatgataagcacaggttttaagatgagtgtcacgacattagtcataaatgtcagtcgtgactcagccaaaatcgcttatcagtcccaatagattatgtcggatgataattatttttaatggcaactcgcgactctcaatctaaagcgccgtcatacgcccccaagagcacgactccagtggcgcaaaaagacagcacacctcggcatcgcaacgccaccactacgcctgaaatgcgactctttatccaacaatcagatctgagcgtgagccagctggcaaagattttaaatattaccgaggcaacggtgcgtaagtggcgtaagcgtgagtccatcagcaacagttcgaacacgccccaccatctcaacacgacgctcacgccgatggaagaatacgtggtggtgggattacgttaccaactcagactcaccttagatagattgctccacgtcacccaaacctacattaatcccaatgtgtcgcgctccggccttgcccgttgcttaaagcgctacggcatatcgcggctcgatgagtttgaagcaccacaagtacccgaacgctattttaatcaattacctgtgactcaaggcagcgatatccaaacctatacggtcaaccccgaaaccttggccaaagccctcgcattgccgagtaccgacggcaatactgtggtgcaagtggtatcactcaccataccgccgcagcttacagagcaagctccaagctcagtgctattaggcatagatacggcaagtgattggatttacctcgatatttatcaagacagcaatactcaagcgaccaatagatatatcgcctatgtcctccgacatgggccgtttcatttacgaaagttgctcgttcgcaactatcacaccttcttagcccgttttcccggtgcccatggcacgccaaagacaaacgcggctgaaccccaaaacaaggtcaccgtatccaggtcgacccgtggagactctttatgagccataccccttctgtacctaattctgcgactgagtcaaaaaaagataaacgactcaacaaacgtttgaaagatatgcccgttgccatcgtcggcatggccagtatcttcgccaactcgcgttatttaaataaattttgggatttgatcagcgaaaaaatcgatgccattactgacatcccagacacccattggcgcgccgaagattactacgatgcagacaaaagcaaagccgataaaagctactgtaaacgcggtggttttttgcctgaagtcgacttcaacccaatggaatttggtctgccgcccaatattcttgagctaacagatacctcgcaactgctgtcgctgattgtcgccaaagaagtgctcgccgatgccaatctgcccaccgattatgatcgcgatcgtatcggcatcacgctaggcgtgggcggtggtcaaaaaatcagtcagagcttgaactcgcgcctgcaatatccagtattgaaaaaagtattcaaaagcagcggcctgagcgatgaagacagcgaaatgctgatcaaaaagttccaagaccaatatatccactgggaagaaaactctttcccagggtccttaggcaacgtgattgcaggccgtatcgccaaccgttttgattttggcggcatgaactgtgtggtcgatgctgcctgcgcgggctcacttgccgctatgcgtatggcgttgactgaactgaccgaaggtcgcagcgacatgatgatcaccggcggcgtctgtaccgacaactcgccgtccatgtatatgagcttctcaaaaacgcctgccttcactaccaatgaaaccattcaaccctttgatatcgattcaaagggcatgatgatcggcgaaggtattggcatggtagcacttaagcgccttgaagatgccgagcgcgatggcgaccggatttatgccgtcatcaaaggcgttggcgcctcatcggacggtaaatttaagagtatttatgcgccgcgccctgaaggccaagctaaagcattggagcgcgcctacgacgacgcgggttttgccccgcacagcattggcttagttgaagcccatggcacgggcactgccgcaggtgatgtggccgaattcaatggcttaaaatcggtatttgcccaaggcaacgacaccaatcaacatatcgcgttaggttcagtgaaatcccaagtgggccacactaaatcaaccgcaggtactgctggggtaatcaaagccgcgctggcgctgcaccacaaggtattacctgcgaccattaacgtcagcaagcctaatccaaaactgaatatcgaaagctcaccattctatttaaataccgaaacgcgcccttggctgcaacgcactgacggtacgccgcgccgtgctggcataagttcctttggttttggcggcactaacttccatctcgtattagaagaatacaaacccgagcacagccgtgacgagcaatatcgtcagcgcagtgtgccgcaaacgctattatttgccgccgccaataaagccgcgctgcttagcgagttaaaagctgcgctgagccaaagcttgaatacgaacgcgaataagagcagcgctgcgagcctgaacgctatcgctcagcaatatccgctacgcgcgctggcagacacagatgcccgtttaggttttgtggctaaggatatcgctcagctgcaagctcagttgaatcaagccatctctcatctagaaagcagtgcgcatttagaaagcgggctcctagataccagtgccagcgaggcgtggcagttaccttctgggatcaactaccgcgcaaaagctttagttgctaaagatgaatcaaagaaagtggccgctctgtttgccggtcaaggttcacagtacctgaacatgggccgcgaactcgcctgccatttccctgaaatgcgccaacaagtgatggccagcgataaggtatttgctcaccacggccaaacgccgttatccaacatcctttatcctattccggcattcgatgccgatgcggttaaagcccaagaagcggcgctgaccaataccctgttcgcccaaagcgccataggcgcggtttcaatggcgcaatattcactattaactcaggcaggttttgccccagatatggtggcgggtcacagctttggtgaattgtcagccctttgcgcggctggcgtgatttcgagtgttgattacgtcgaactggccttcgcccgtgggcacgccatggcgcaagtgccaagtgatactgacgctcaagtcgatttaggcaagatgtttgccatcatcctcaaacaaaagagtgatatcgatgcgcttaatcgctgtttagctcagtttgatggcgttaaaattgccaactacaacgcgccgacacaattagtcatcgcaggtggcacagagcaaactcagctggctgccaaagctattagcgaacagggctttaaagcgattgccctgcccgtctctggcgcgttccacacccccttggttgggcacgcacaaaagccatttgccaaggccattgataaagctaaattcagcgcgccttcaatcgcactttatgccaacggcacaggtcaattgcacccaagcgatggcaaagcgattaaagcagaattcaaacaacacatgctgcaatcggttcgttttagcgaacaactgcaggccatgtatgatgctggcgcccgtgtgtttgttgagtttggccctaagaacatactacaaaagctcgttgaaaataccttaagtgaacacttaaatgagctttgccttgtcagcatgaacccgaaccctaagggcgacagtgacagtcaattacgcttagccgctgtgcaactcgcggtagccggtgtggcgttaactgaggttgacccctatcaagccgtcacgtcacaggagattgccgagcgtgaagcgccatcggcgatgaacatcaaactcaccgccactaaccacatcagtgccgcaacccaagctaagatggcgaaatctctcgccacaggcagcgtgactagccaagtgcaatatgtggatcgcatcgttgaaacgattctcgaaaaagaagtcgaaaaaatcgttcagaaagaagtcatagtcgaaaaagtggttgagaaaataatagaagtggaagcgaatcaagtggcagctgttgaaatgaaacaaaaactcctaagcgtgacgcaaggcttaaatcaccaacaagcgacggcgcagatgagcccaagcacagcaaacgtcagcggcgatgcgttaacggcatttttcagcgcacaatctcaagcggcgcagttacatcaacagtttttagccatcccgcagcaatatggcgatactttcacgacactgatgacagaacaagccaagatggcgagccttggcattgcaatcccagaaagcctgcaacgctcgatggaaatgttccaccagcatcaagcgcaaactctgcaaagccacgccgaatttatgcaactgcaaagcagtagcagccaagcggccttggccatgttaaacaatgcgccgattaactttaccccagcggttgcaagtcagccacaagcaactgtacctgttgctcctgcacctgttgctgcatcgacagtggcacacaatgccgcaccagtcgcggctcaggctgttgcgacaagaccagcggttagcacacccgttcctcctgttgtgcaaaccgctcccgttgcttatgcgcctgctgcgacagtacaagttgcgccagcagctcctgccgcacccgcattagtcatgcctgctgtggtgatgccagaagtgacacctgtggcaccaacaatgaacggtttaagtgctgaaaaagtacagcaaacaatgatggctgtggtggctggcaagacaggctatccgactgaaatgctggagctcagcatggatatggaagccgatctcggtatcgattcaattaagcgcgttgaaatcttaggcacagtgcaagacgagctgccgagtttacctgaactcagcccagaagatttggctgagtgccgtaccttgggtgaaatcgtaagctatatgaatagcaaattacctgcagcgggaactattgcttcgacagcagcgccagttgctcaaaccattgccgctcctgcatctaacgctttaagcgctgcattagttcaacaaactatgatggctgtggtggccgacaagacaggctaccccactgaaatgctggaacttggcatggatatggaagccgatttaggtatcgattctattaagcgcgttgagattttaggcacagtgcaagacgaactgccgggtttacccgaacttagcccagaagatttggccgaatgtcgcaccttaggtgaaatcgttacctatatgaactctaaactgccagcagcgggttcagttgtagtttctacaacagcacaagccgcgcctgccgacagcggtttaagcgctgagttagtgcattcttctgaaatcgagagcaccatgatggctgtggtggccgacaagacaggctatccgactgaaatgctagaactgagcatggatatggaagccgatctcggtatcgactccatcaagcgcgttgaaatcctaggcacagtgcaagacgaactgccgggcttacctgaacttagcccagaagatttggccgagtgtcgtaccttgggtgaaatcgttagctatatgaatagcaaattaccagcagcaggagttattgcttcgacagctgcgccagttgctcaaaccattgcagctcctgccgcaatcggtttaagcgctgcattagtgcaacaaactatgatggctgtggtggctgacaagacaggttaccccactgaaatgctggaactcagcatggatatggaagccgatttaggtatcgattctatcaagcgcgttgaaattttaggcacagtgcaagacgaacttcctggcttacctgaactcaatcctgaagatctagccgagtgtcgcaccttaggtgaaatcgttgactacatgaacagcaaactacctgcggttggctcgacttcaactataacagcagcacaggtcactgttaccgctacaataaataacggcttaagtgctgaaaaaatccaacaaaccatgatgtcagtagtggccgacaagacgggctaccccactgaaatgctggagctcagcatggatatggaagccgatttaggtatcgattctatcaagcgcgttgaaatcctaggcacagtacaagacgaactgccgggtttacctgagctaaatcctgaagatctggccgagtgtcgcaccttaggtgaaatcgttatgctcttttcgcaagatttagggcaaggcaagctagaccagaataatgcggcgaatgtggtaaacgtcgctgtcgcagttgaaccgactatcgatttgctaccccatgatttaccacaacatgatttaccaccacacagtgaggtggtgctaaaaaagttgccagcggcggctgagctaacgcaactatcgccacagcaatcgtcaaagcaatcagcgcaacaagctcaaacacgcgtttttgctaaagatgcctgcattattattagcgatgatggtcacaatgctggcgtacttgccgagaaattgcatgctcaaggtctaacggttgcggttgtgcgctcgccagaaagccttgtagccagtgcatcaccgctcaatagccacattgccagcttcacattggcggcgattgacgatatcagcatcagcgtagtaattaatgagatcaaaaccttaggtcaagttgcaggctttattcatctgcaaccacaacataaaacctcagccgatgcgaaaggtttagtgttgtccagtgccgccaaagcgtcggtcgagcaagcgttcttgttcgccaagcacttacagccgcttttaacaacagcagcaatcgccaatactggcagcagctttatcagcgtcagtcgtatcgacggtggttttggttatcttaaccacagccaaattgcccgcagcgagttaaaccaagcggcattggcaggtctgactaaaaccttgagccacgagtggccaagcgtgcattgccgcgccttagatatcgcgccagcacttgatgccaagcaactggccaatgcggtcatagctgagttattcgcaaccgataagcttttagaagtcggagtgagtgaaagtggtgtgagtgaagctggcgcaactgaaacgctggcacgcgtgaccttagttgcaggcaaggcagatacccgccacggcgcagctaacttgaccagtgctgataaaattctagtcactggcggcgcaaaaggcgtgacctttgaatgtgccttgagtctggcaaaacgcagcaaggcgcactttatccttgccggcagaagcagccaacaaaccattcccgcttgggcacaaggtaaaaataacagcgaacttaaagccgcagctattgcccatattcaaaacttgggcgagaaacctacaccaaaacaagtagatgccttagtatggccagttcaaagcagcctagaaattgcagccgcactagaagcctttactgcagtaggcgcgagtgccgagtatttaagtctcgatgtaaacaatccagacgcgatcgccagcacgatagtgccgatcaccgagttatcacctattaccggcatcatccatggtgcgggcgtactcgccgataaacacatccaagacaaaaccttagatgagttagcacgggtgtatggcactaaggtcacaggcattagcaatctgcttgccgcactggatttagataaagtaaaactgattgccctgttctcttcggcggcgggtttttacggtaataccggccaaagcgattacgccatgtccaacgacatactcaacaaagccgcactgcaactggcgcagcaactgccaaatgccaaagtgatgagcttcgattggggtccgtgggacggcggcatggtgaatcctgcgctgaaaaagatgtttatggatcgcggtgtttatgtaatcccgctaaaagcaggtgctgagttatttgcgagccaattactgagcaacacaggcgcacaattgctggtcggtactgacatgcaaggcagcgcacctcatgacgacacgcctaacgaagtacaagaaactgaaggtagtaatctaaaaaagcctgaagcggatctgaccactgactcgtcggatccgcatgctttgcttaacgctttaaatccaagcgcagtaaatataagcgctgtaaaactgcagcgcacactggaccctaaggcaatgatctttattgaagatcactgcattaacggtaatcccgtattaccgactgtgtgcgcgattcagtggatgcgtgaagccgcctttgatgtgctcaagcaaccagtcaaagtgcaaagctacaagctgttgaagggcattattttcgacgctatgacgttagaaaacggcgcgcccatcacgctagaacttgagcttgcaccgattgcgttaacggataaagccgcgaaagacacagacgagtacttgagtggacaattcagtgccttaatcagctttgaaggtcgtccgcaatatcaagccatcttagtgattgatgatgctcctagtgataacttagccactaatagcaaagcaactgcctttgacgcgcatagcttggcaggattttctgccatcacaaccgcaagcagcctttacagcgacggcacgcttttccacggcccaagactgcaaggtattgagtctgtggtgaagtttgacgatgcgagcttagtcgccaaggtgagtcttccccatgttgcgttagcggactgcgggagttttgtgcccaatctggcccctaaaggctcacaagcttttgcagaagatttgttgctgcaggcgatgctggtatgggcaagactcaaatatggcgcggcgagtttgccatcaagcattggtgagtttatctcccatgcgccgtttgcctttggcgatacagggtatctagtgctcgaggtggtaaaacactctggccgtgcccttgaagccaatattgcactttatcatcaagatggccgcttgagctgcgagatgaacaacgccaaagtcaccatcagcaaaaaccttaacggtgcattcttagcgaataaagttgctaataaaagcattgaaagcgtggaggcaaaagtcgagtgaacttagcctatcctttagcaatgacccataacggccaagctgaaacgtcgatagctgacaaggctattgctgacaaaagcgccaagccactgcgtattgcagtgttgcttggcgatgcggtcaaccttgactctcactcagcccaagtattagggacgtttactgaacgtgaaagagttcaaatttgcgctgccgatgccaaccaatcgacagtacataggccgacagctcatgaacccaaagagcatgaaccgtcagtgtatgagcaaaggtcgctcacagccttgttaggccaagcaacaacagccattgagcaaggcaagctcgttgaacttaaatttgaggatggtaatctaccccagtatctgtatttgctcgatggtttacgcgccgccaaactgcgtcttcacgcccacgcgtttattgctggctttgccgctggcaatgaggttacagatgttgcaaatgcggcgactgttgcaaataaggcgttagcggcagcaaagcgcagcccagctcaaaccgttcaacatcagactgtcgccaacacactcaacgaggcatttgttgcgcttcgccaaggcgtaaccgccctcgccgcgcgaacacaagcgccgcttaaaggcactactggtataaaacagacaaacgacaccaatcatcaaactggctattggtttagcgaccaacatcaagcgcgggtgttgtgtctaaatcttgtagcaaagacgtcacatcaagcggatgagtctcgaaatctaagccaaagcctagtgctgacccaaggcacacaactcgccgcgcccaaagcccttgtcgatgaaaaccggctgtttgtgccgataagtagtgacagtattaatgagttaaaagcaaagctgtttcagttgcttagttcactagacattggcgcaccagacacgtcatctgcatcacatcaactcacattctggtttgaacgctacgatgccaacgcaccactggcattagtcttgatggcggcatcaagtgatgatctcaaacttgaagccaaagccatgcttacggcgcttgaaaatgatgctattcgtcatcatggtcaacactttaagacaccagcgggtagctgctttaccgctaagccgctaggggatgcgggactgacttttgtgtatcctggcgttggcacggtttacgccaatatgttcaataacttgcatgagtatttccccgcgctgtatcaccagttagaacgtgaaggcgatttaagcgccatgctgcaatcaccgcagatttatgcggcagatgttaaaaccgcggctggcatgaacctaagtcagcaagcgattagtggcgtgggggccagttatctattcactaaactgttgacccaagtctttaatattaagcctaaaatggcgctcggttactcaatgggcgaagcagccatgtgggccagcctagatgtgtggcaaacaccgcacgctatgattaacgccactgaaaatagcgatattttcaaccatgcgatttctggtgagctaactgctgtgcgccgagcatggcagcttgcggatgatgaagccatagtgtggaacagctttgtggtgcgcgctgatagccatgaaatcaaggcattattgccagagtttcctcgcgcctacttagccatcacccaaggtgatacttgtgtcattgcaggctgcgaaacaagctgtaaagccctgcttgccacgttaggcaaacgcgggattgccgccaatcgcgtcacggcaatgcataccgcgcctgccctgttagtccatgggcaagtacaagatttctatactcaagcgctaaaacctgaggcactggaacctgatgcgctgaaagcggcagcgcaagattcgtctgttcgctttattagcgcagcgcaaactgcgccagtaatcgtggatagccacagcattggccgcgcgattgccgataccttttgttcgccactcgactttagtgcgctcattcaaaatgccactgagcaaggcgcaaggctgtttgtcgaagtgggcgccgataggcaaaccagtacactcatagataaaatcagccatgcccacgcaagccaaagctctgcgaacgcggcgacagccgccattgcctgcaatgccaaaggtgccgacgcgatcaccagcttactcaagtgtttagctcagctaataagccacagagtgccgctttcgctcacgccgcttattcagccattaagtgctaacgcagcccctttatcatcagcagtatcaccaaaaggagaaccccagtgagttctcagcattcccctacgattgataaaactaccgtgcctaccattgcgtcaaaccgcgcatcaaaaagcgcgtcaaaaattgcgatcgtcggcctcgcgactcagtatcctgacgccgataatcctcaaaccttttggcaaaatctgctggataaaaaagactctcgcagtcaaattagccgcgagaagctcaatgccaatcccgccgattaccaaggtgtgcagggtcaatctgaccgtttttactgtgataaaggcggctacatccaacacttccagtttgatgccaagggttatcaactgcctgagtccgcctttgacggtttagatgaaagctttttatgggcactcgattgcagtcgcaaagccctccaagacgcagggattgccccaagcgatgccgtactggcgcgcacaggtatcgtgatgggaaccttgtcgttccccacagcccgctccaacgaattatttttgcctctgtatcatcaaaccgttgaaaaggcgctgcaaaacaaactgaatcaaagcacttttcagctggctgattttaaccaagctcacgccgacaaagcattaaacgtcgagcaagcattaaacgtcgccaatggcgccgttgcccacacagcttcaaagctagtcagcgatgcactcggtttaggtggcactcagttaagcctggacgctgcctgcgctagctcggtttacgcacttaaactcgcctgcgattacctgaccacaggcaaggccgatatgatgcttgctggcgcggtatcgggcgcagatcccttctttatcaacatgggattctcgatttttcacgcctatcctgatcatggaatctcagcaccctttgatagcaacagcaagggcttattcgccggagaaggcgcaggggtgttagtattaaaacgcttagaagatgccgagcgcgatggcgataacatctatgccgtcgtcagcggcattggtttgtcgaacgacggtaaaggccagtttgtattaagccccaacagcaagggccaagtgcaagctttcgagcgcgcctatagtgcggcaaacacactgcccgccaatatcgaagtgattgaatgccacgccaccggcacgccgcttggggataaggtcgaactcgcctcgatggaacgtttcttcgaggacaaactcgcgggctctgcagtgccgctgatcggttcggcaaaatccaacttaggccatttgctcacagccgcaggcatgccggggatcatgaagatgatttttgccatgcgctcgggtcgactgccgccaagtattaacttatcagcgccgatatcctcgcctaagggcttgtttagcgaaaagaatctgccaacagaattacatgcttggcccgataaagccggaaactcccgccgccacgccggtgtttccgtgtttggttttggcggctgtaacgcgcatttgttgctggaatcctatgttgccaatacaaacaaaaagaatgaacaagccgcagctgctgtaagttatcagcacacgccattaaatatcattggcttagcgtcgcacttcggccctttatcctccattaatgcactggatagcacgattcaagctcggcaacatgcctttatcccgctgcccgctaaacgctggaaaggcttagacaaacaccctgatattctggccaacgttggtttaagcggaactggcctcgcagccgcaccacagggcgcgtatatcgaccagttcgattttgacttcctgcgttttaaagtgccgcccaatgaagatgaccgcctgatctcccagcaactgctgttgatcaaagtggcagatgaagcgattcgtgatgccaaccttaagcccggtggcaaggttgctgtattagtggcgatggaaactgaactcgaattacatcagttccgtggccgcgtaaatctgcacacgcaactggcagacagtcttaaaaagcaagggatcaccctcacacaagccgagtatctcgcccttgaaaaaatcgccatggacagtgtgctcgatgccgccaagctgaaccaatacaccagttttattggcaacatcatggcgtcacgcatcgcatccctttgggactttaatggccccgcctttaccatttcggcggcggaacaatcggtcgcccgctgtatcgatgtggccgaaaacctcttgtcccaagaatccttagatgccgtagtgatcgccgccgtcgatttaagtggcagtctagagcaagtgatcctcaaaaacgccgtatcaccagtggcatttaatgccactgacactggttggaaagtcggtgaaggtgcaggcgcactggtgctaactgctgaaaattcaaatactaatgctctacttaataatgccaatagcaacagctatggtcacatcagcggccaagtatttggcgcgatttgtgacatgcaaggtaacagcaacacagcgcgtatttgcgatgacttactaacccaagccaaggtgaatagcagccagattagcttgattgaaaccagtattgcggttgagcaacttgccgattcagagctggtactcaataccctgctgccgagtgtgaaccagcgcagccaagccgctgataccctaggccacaatttgccgcagcgggaatggcgagtattttgagcgccctgcttcagcttaaaaatcaagggcaattaaaaaaccaagcacagcaacaagctaatcaagtgcagcacgcgctcgttgccacgtttagccaaggtaaatgctcgcagttattgctcagtcaaagtgcgacgcaagcacacagtttgcagcaaaggcttgaacaagacttaacgctttctgagcaaaaacacttaattaaacaagtgacacttggtggccgcgatatctatcagcatatccttgatacgccgttggcggacatagatgcaataaagcaaaaagcccaagccatcactgcattgccaaagcgcagccaacgcaaacatttggcccaaatagcgagcaaagacacgagtggctttgcaacaagcagcccaaccacggctctacaaaaagagacattaagcagcatgccaattaatgccctaagcacgcccaatgacaacgcagctcaaacagagctaaaagacgctgcatttatacgtaatcagcaactcgcccgtgaagcccatttagcctttttacaaagccgtgcgcagggcctgaaactggccgatgccttgatgaaagcccagcttgccagcgagttagccgtcaatggccaagcgacgccggtacaacagcaagccactgttcaggcgccagtgtatgcatctgctcatacacctgagctagctccagttgttaactcagaggcaaacccagctgcgctttatccaaaccatgcaaaagtgcctctgtacacgccgccaacaccgataagcaagccttgcatttgggattatgcggatttagtcgagtacgccgaaggcgatattgctaacgtctttggccaagattacgccattatcgacagctattcgcgccgtgtacgcctgccaacgactgactatttgctggtatctcgggtaacgaaattaaacgcccagatgaaccaatatcagccttgcactatgaccacagaatacgacattcctgtggacgcgccgtatttggtcgatggccaaattccttgggccgtagcggttgaatcgggtcaatgcgacttgatgctgatcagctacttaggtatcgactttgaaaacaagggcgagcgcgtttatcgcctgctcgattgcaccctgaccttccttggcgacttaccgcgcggtggcgacaccctgcgctacgatatttcaatcaatcactttgcccgcaatggcgataccttgttgttcttcttctcctacgaatgtttcgtgggcgacaagctgatcctgaaaatggatggcggctgtgccggcttcttcaccgataaagaactagccgacggcaaaggcgttattcacaccgaagccgaaatcaaagcgcgcaacctcgccttgaacaatccgaataagccgcgctttaatccgttactcaactgcgcgcaaaaccaatttgattacagccaaatccataaactgctcggcgccgatatcggtggctgttttggcggcgcacacgcggcgcatcaagcccaatatggtttgcagccctctttatgttttgcatctgaaaaattcctgatgattgaacaagtcagcaatcttgaggtgcatggcggcgcgtggggcttaggctcagttcaaggccataagcagctcgaagccgatcattggtatttcccgtgtcatttcaagggcgaccaagtgatggcggggtcgttaatggccgaaggctgtggtcaattactgcaattctttatgctacatattggtatgcacctcggtgttaaagatggtcgtttccaaccgctcgaaaacgcgtcacaaaaagtgcgttgtcgcggtcaagtgttgccgcaatcaggcctgctcacctatcgtatggaaatcactgaaatcggtatgagcccgcgcccgtatgctaaggcgaatatcgatattctgctcaatggtaaagtggttgtggacttccaaaaccttggggtgatgatcaaagaagaagccgaatgcacccgctaccttgcggataatgatgccagcacagctgacaatacgactaaaaatgctgccaaaaatgctgcttcggctgtgccgctagtttcgacaacacccgcatcgttcgccgcgccgttgatggcccagctgccagatttaactgcgccaaccaataaaggcgtagtgccgcttaagcatgtgcctgcgccgattgcgcaaacggattcaaagtacgccaaccgcgtgcccgataccctgccgttcacgccgtaccacatgttcgaatttgccacgggcgatatcgaaaactgcttcggccccgatttcagcatctatcgcggccttattccaccgcgcacgccttgcggtgatttacagcttaccacccgcgtcattgcgattgacggcaaacgcggcgagctgaaaaagccttcttcgtgtatcgccgaatacgaagtgcccgcaaacgcttggtattacgataaaaacagtcatcatgctgtgatgccctattcagtgctaatggaaatatcactgcagccaaatggctttatttcaggctatatgggcaccaccttgggcttccccggccaagagctgtttttccgtaacttagacggcagcggtaagctgctgcgccacgtggatttacgcggcaaaaccatagtgaacgactcacgtttgttatcgactgtgattgccggcagcaacatcatccagaatttcagcttcgagttaagctgcgatggcgagcctttctaccaaggtaaagcggtatttggttacttcaagggcgatgcgctgaaaaaccaactcggcatagacaacggcaagatcacacagccttggcatgtgcaaaatggcatagccgccgatagccaaatcaatctgttagataaacagcatcgcagctttaacgcgccagaaggtcagccgcattaccgtttagcgggcggtcagcttaactttatcgacaaggccgacatagtgaaagccggcggtaaagcgggccttggctatttatacgccgagcgcaccattgacccgagtgattggttcttccaattccacttccatcaagatccggtaatgccaggctcattaggggttgaagcgattatcgagctgatgcaaacctatgcgattgaccaagaccttggtgcgggctttaagagtccaaaattcggccagatattatcggatatcaaatggaagtatcgcggccaaatcaacccattaaacaaacagatgtcgctggatgtgcacattaccagcgtgacagacgacaatggcaaacgcatcattatgggcgatgccaacttgagtaaagatggtctgcgaatttatgaagtcaaagatatcgccatctgtattgaagaagcttaatcaccttgctcatttagaaagacgctaattacaaatactgtgtcgccttggccagaatgcctaaggcggcaataaaaagagaatacatatgacaagccatactctcgatcaatttaatagtaataacgaaaaactcagcccttggccgtggcaagtcaacgatgccgcgctgagctttgatatcgactcattaggcaaaaaactcaaagatttaagccaagcctgttacttagtgaatcacagtgaaaaaggcttaggcatagcgcaaacagccgaagtaaccacaagcgacagccaagcgccactaggctcacaccccgtcagcgcctttgcgcccgcccttggcacccaaagtttaggcgacagtaattttcgccgcgtacacggggttaaatacgcttactacgctggcgctatggctaacggtattgcctcagaagaactggttatcgcgctgggccaagcgggcattttgtgttcgtttggcgcggcggggttaatcccatcccgcgttgaagcggccattactcgcattcaagcggcgctgcctaatggtccttacgcctttaatttaattcacagcccaagcgagcccgcattagagcgcggcagtgttgagttgttcttaaaacataaagtgcgcacggtcgaagcctcggcatttttaggtttaacgccacaaatcgtctattaccgcgcagcaggtttgagccgcgacgcacatggcgacatcgtcattggcaacaaagtcatagccaaaatcagtcgcaccgaagtcgcgactaagtttatggagccggcgcctgccaaaattctgcagcaattagtcagtgaaggccttatcagccaagatcaaatggcgatggcgcaacttgtacccatggcggacgatatcacggccgaagccgattctggcggccataccgacaatcgtccactggtcacgctattgccgacgattttggcgctcaaagatgaaatccaagctaagtatcaatacaagacgcccatccgtgtgggagcaggcggcggcgttggtacccccgacgcagcattagccaccttcaacatgggcgcggcctttatcgtcacaggttcaatcaaccaagcgtgtgtggaggcgggcgcgagcgaacacacacgtaagttactcgccaccacagaaatggccgatgtgactatggcacccgccgccgatatgttcgaaatgggcgtgaaattacaagtggttaagcgtggcactctgttcccgatgcgcgccaataagctttatgaaatctacacccgttacgattcaattgatgccatccccgcggacgagcgtaaaaagctcgaagagcaagtgtttcgcgcatcactcgatgacatttgggcgggcactgtcgcccactttaacgagcgcgatcctaagcaaatcgagcgcgcactggataaccccaaacgtaagatggcgttgattttccgctggtatttgggtctgtctagccgctggtcgaacacaggtgaagtcggccgcgaaatggattaccaaatttgggcaggccctgcccttggcgcatttaatgcgtgggcaaaaggcagttatttagatgactataaagcccgtaatgcggtcgatttagccaaacatttaatggtgggcgcggcctatcaatcccggattaacttgctgttatcccaaggggttagcattccagttagcctgcaacgctggaaaccgctaaatcgtttttaacccctaacggactatcgagacatcaagtagggactcaactccctacttgatgcaatcttcttcactacctcgttaaatcgaagccgccgaccttattaccaatctaatcccttgtttgtgccaatgccgctaagcactgtcgctccagtggcacgccgtaaacccatccttgggggctcaacggaaaagtccctttttccgatgg

SEQ ID NO: 2 - DNA, 8170 bp, Shewanella baltica
atgagccataccccttctgtacctaattctgcgactgagtcaaaaaaagataaacgactcaacaaacgtttgaaagatatgcccgttgccatcgtcggcatggccagtatcttcgccaactcgcgttatttaaataaattttgggatttgatcagcgaaaaaatcgatgccattactgacatcccagacacccattggcgcgccgaagattactacgatgcagacaaaagcaaagccgataaaagctactgtaaacgcggtggttttttgcctgaagtcgacttcaacccaatggaatttggtctgccgcccaatattcttgagctaacagatacctcgcaactgctgtcgctgattgtcgccaaagaagtgctcgccgatgccaatctgcccaccgattatgatcgcgatcgtatcggcatcacgctaggcgtgggcggtggtcaaaaaatcagtcagagcttgaactcgcgcctgcaatatccagtattgaaaaaagtattcaaaagcagcggcctgagcgatgaagacagcgaaatgctgatcaaaaagttccaagaccaatatatccactgggaagaaaactctttcccagggtccttaggcaacgtgattgcaggccgtatcgccaaccgttttgattttggcggcatgaactgtgtggtcgatgctgcctgcgcgggctcacttgccgctatgcgtatggcgttgactgaactgaccgaaggtcgcagcgacatgatgatcaccggcggcgtctgtaccgacaactcgccgtccatgtatatgagcttctcaaaaacgcctgccttcactaccaatgaaaccattcaaccctttgatatcgattcaaagggcatgatgatcggcgaaggtattggcatggtagcacttaagcgccttgaagatgccgagcgcgatggcgaccggatttatgccgtcatcaaaggcgttggcgcctcatcggacggtaaatttaagagtatttatgcgccgcgccctgaaggccaagctaaagcattggagcgcgcctacgacgacgcgggttttgccccgcacagcattggcttagttgaagcccatggcacgggcactgccgcaggtgatgtggccgaattcaatggcttaaaatcggtatttgcccaaggcaacgacaccaatcaacatatcgcgttaggttcagtgaaatcccaagtgggccacactaaatcaaccgcaggtactgctggggtaatcaaagccgcgctggcgctgcaccacaaggtattacctgcgaccattaacgtcagcaagcctaatccaaaactgaatatcgaaagctcaccattctatttaaataccgaaacgcgcccttggctgcaacgcactgacggtacgccgcgccgtgctggcataagttcctttggttttggcggcactaacttccatctcgtattagaagaatacaaacccgagcacagccgtgacgagcaatatcgtcagcgcagtgtgccgcaaacgctattatttgccgccgccaataaagccgcgctgcttagcgagttaaaagctgcgctgagccaaagcttgaatacgaacgcgaataagagcagcgctgcgagcctgaacgctatcgctcagcaatatccgctacgcgcgctggcagacacagatgcccgtttaggttttgtggctaaggatatcgctcagctgcaagctcagttgaatcaagccatctctcatctagaaagcagtgcgcatttagaaagcgggctcctagataccagtgccagcgaggcgtggcagttaccttctgggatcaactaccgcgcaaaagctttagttgctaaagatgaatcaaagaaagtggccgctctgtttgccggtcaaggttcacagtacctgaacatgggccgcgaactcgcctgccatttccctgaaatgcgccaacaagtgatggccagcgataaggtatttgctcaccacggccaaacgccgttatccaacatcctttatcctattccggcattcgatgccgatgcggttaaagcccaagaagcggcgctgaccaataccctgttcgcccaaagcgccataggcgcggtttcaatggcgcaatattcactattaactcaggcaggttttgccccagatatggtggcgggtcacagctttggtgaattgtcagccctttgcgcggctggcgtgatttcgagtgttgattacgtcgaactggccttcgcccgtgggcacgccatggcgcaagtgccaagtgatactgacgctcaagtcgatttaggcaagatgtttgccatcatcctcaaacaaaagagtgatatcgatgcgcttaatcgctgtttagctcagtttgatggcgttaaaattgccaactacaacgcgccgacacaattagtcatcgcaggtggcacagagcaaactcagctggctgccaaagctattagcgaacagggctttaaagcgattgccctgcccgtctctggcgcgttccacacccccttggttgggcacgcacaaaagccatttgccaaggccattgataaagctaaattcagcgcgccttcaatcgcactttatgccaacggcacaggtcaattgcacccaagcgatggcaaagcgattaaagcagaattcaaacaacacatgctgcaatcggttcgttttagcgaacaactgcaggccatgtatgatgctggcgcccgtgtgtttgttgagtttggccctaagaacatactacaaaagctcgttgaaaataccttaagtgaacacttaaatgagctttgccttgtcagcatgaacccgaaccctaagggcgacagtgacagtcaattacgcttagccgctgtgcaactcgcggtagccggtgtggcgttaactgaggttgacccctatcaagccgtcacgtcacaggagattgccgagcgtgaagcgccatcggcgatgaacatcaaactcaccgccactaaccacatcagtgccgcaacccaagctaagatggcgaaatctctcgccacaggcagcgtgactagccaagtgcaatatgtggatcgcatcgttgaaacgattctcgaaaaagaagtcgaaaaaatcgttcagaaagaagtcatagtcgaaaaagtggttgagaaaataatagaagtggaagcgaatcaagtggcagctgttgaaatgaaacaaaaactcctaagcgtgacgcaaggcttaaatcaccaacaagcgacggcgcagatgagcccaagcacagcaaacgtcagcggcgatgcgttaacggcatttttcagcgcacaatctcaagcggcgcagttacatcaacagtttttagccatcccgcagcaatatggcgatactttcacgacactgatgacagaacaagccaagatggcgagccttggcattgcaatcccagaaagcctgcaacgctcgatggaaatgttccaccagcatcaagcgcaaactctgcaaagccacgccgaatttatgcaactgcaaagcagtagcagccaagcggccttggccatgttaaacaatgcgccgattaactttaccccagcggttgcaagtcagccacaagcaactgtacctgttgctcctgcacctgttgctgcatcgacagtggcacacaatgccgcaccagtcgcggctcaggctgttgcgacaagaccagcggttagcacacccgttcctcctgttgtgcaaaccgctcccgttgcttatgcgcctgctgcgacagtacaagttgcgccagcagctcctgccgcacccgcattagtcatgcctgctgtggtgatgccagaagtgacacctgtggcaccaacaatgaacggtttaagtgctgaaaaagtacagcaaacaatgatggctgtggtggctggcaagacaggctatccgactgaaatgctggagctcagcatggatatggaagccgatctcggtatcgattcaattaagcgcgttgaaatcttaggcacagtgcaagacgagctgccgagtttacctgaactcagcccagaagatttggctgagtgccgtaccttgggtgaaatcgtaagctatatgaatagcaaattacctgcagcgggaactattgcttcgacagcagcgccagttgctcaaaccattgccgctcctgcatctaacgctttaagcgctgcattagttcaacaaactatgatggctgtggtggccgacaagacaggctaccccactgaaatgctggaacttggcatggatatggaagccgatttaggtatcgattctattaagcgcgttgagattttaggcacagtgcaagacgaactgccgggtttacccgaacttagcccagaagatttggccgaatgtcgcaccttaggtgaaatcgttacctatatgaactctaaactgccagcagcgggttcagttgtagtttctacaacagcacaagccgcgcctgccgacagcggtttaagcgctgagttagtgcattcttctgaaatcgagagcaccatgatggctgtggtggccgacaagacaggctatccgactgaaatgctagaactgagcatggatatggaagccgatctcggtatcgactccatcaagcgcgttgaaatcctaggcacagtgcaagacgaactgccgggcttacctgaacttagcccagaagatttggccgagtgtcgtaccttgggtgaaatcgttagctatatgaatagcaaattaccagcagcaggagttattgcttcgacagctgcgccagttgctcaaaccattgcagctcctgccgcaatcggtttaagcgctgcattagtgcaacaaactatgatggctgtggtggctgacaagacaggttaccccactgaaatgctggaactcagcatggatatggaagccgatttaggtatcgattctatcaagcgcgttgaaattttaggcacagtgcaagacgaacttcctggcttacctgaactcaatcctgaagatctagccgagtgtcgcaccttaggtgaaatcgttgactacatgaacagcaaactacctgcggttggctcgacttcaactataacagcagcacaggtcactgttaccgctacaataaataacggcttaagtgctgaaaaaatccaacaaaccatgatgtcagtagtggccgacaagacgggctaccccactgaaatgctggagctcagcatggatatggaagccgatttaggtatcgattctatcaagcgcgttgaaatcctaggcacagtacaagacgaactgccgggtttacctgagctaaatcctgaagatctggccgagtgtcgcaccttaggtgaaatcgttatgctcttttcgcaagatttagggcaaggcaagctagaccagaataatgcggcgaatgtggtaaacgtcgctgtcgcagttgaaccgactatcgatttgctaccccatgatttaccacaacatgatttaccaccacacagtgaggtggtgctaaaaaagttgccagcggcggctgagctaacgcaactatcgccacagcaatcgtcaaagcaatcagcgcaacaagctcaaacacgcgtttttgctaaagatgcctgcattattattagcgatgatggtcacaatgctggcgtacttgccgagaaattgcatgctcaaggtctaacggttgcggttgtgcgctcgccagaaagccttgtagccagtgcatcaccgctcaatagccacattgccagcttcacattggcggcgattgacgatatcagcatcagcgtagtaattaatgagatcaaaaccttaggtcaagttgcaggctttattcatctgcaaccacaacataaaacctcagccgatgcgaaaggtttagtgttgtccagtgccgccaaagcgtcggtcgagcaagcgttcttgttcgccaagcacttacagccgcttttaacaacagcagcaatcgccaatactggcagcagctttatcagcgtcagtcgtatcgacggtggttttggttatcttaaccacagccaaattgcccgcagcgagttaaaccaagcggcattggcaggtctgactaaaaccttgagccacgagtggccaagcgtgcattgccgcgccttagatatcgcgccagcacttgatgccaagcaactggccaatgcggtcatagctgagttattcgcaaccgataagcttttagaagtcggagtgagtgaaagtggtgtgagtgaagctggcgcaactgaaacgctggcacgcgtgaccttagttgcaggcaaggcagatacccgccacggcgcagctaacttgaccagtgctgataaaattctagtcactggcggcgcaaaaggcgtgacctttgaatgtgccttgagtctggcaaaacgcagcaaggcgcactttatccttgccggcagaagcagccaacaaaccattcccgcttgggcacaaggtaaaaataacagcgaacttaaagccgcagctattgcccatattcaaaacttgggcgagaaacctacaccaaaacaagtagatgccttagtatggccagttcaaagcagcctagaaattgcagccgcactagaagcctttactgcagtaggcgcgagtgccgagtatttaagtctcgatgtaaacaatccagacgcgatcgccagcacgatagtgccgatcaccgagttatcacctattaccggcatcatccatggtgcgggcgtactcgccgataaacacatccaagacaaaaccttagatgagttagcacgggtgtatggcactaaggtcacaggcattagcaatctgcttgccgcactggatttagataaagtaaaactgattgccctgttctcttcggcggcgggtttttacggtaataccggccaaagcgattacgccatgtccaacgacatactcaacaaagccgcactgcaactggcgcagcaactgccaaatgccaaagtgatgagcttcgattggggtccgtgggacggcggcatggtgaatcctgcgctgaaaaagatgtttatggatcgcggtgtttatgtaatcccgctaaaagcaggtgctgagttatttgcgagccaattactgagcaacacaggcgcacaattgctggtcggtactgacatgcaaggcagcgcacctcatgacgacacgcctaacgaagtacaagaaactgaaggtagtaatctaaaaaagcctgaagcggatctgaccactgactcgtcggatccgcatgctttgcttaacgctttaaatccaagcgcagtaaatataagcgctgtaaaactgcagcgcacactggaccctaaggcaatgatctttattgaagatcactgcattaacggtaatcccgtattaccgactgtgtgcgcgattcagtggatgcgtgaagccgcctttgatgtgctcaagcaaccagtcaaagtgcaaagctacaagctgttgaagggcattattttcgacgctatgacgttagaaaacggcgcgcccatcacgctagaacttgagcttgcaccgattgcgttaacggataaagccgcgaaagacacagacgagtacttgagtggacaattcagtgccttaatcagctttgaaggtcgtccgcaatatcaagccatcttagtgattgatgatgctcctagtgataacttagccactaatagcaaagcaactgcctttgacgcgcatagcttggcaggattttctgccatcacaaccgcaagcagcctttacagcgacggcacgcttttccacggcccaagactgcaaggtattgagtctgtggtgaagtttgacgatgcgagcttagtcgccaaggtgagtcttccccatgttgcgttagcggactgcgggagttttgtgcccaatctggcccctaaaggctcacaagcttttgcagaagatttgttgctgcaggcgatgctggtatgggcaagactcaaatatggcgcggcgagtttgccatcaagcattggtgagtttatctcccatgcgccgtttgcctttggcgatacagggtatctagtgctcgaggtggtaaaacactctggccgtgcccttgaagccaatattgcactttatcatcaagatggccgcttgagctgcgagatgaacaacgccaaagtcaccatcagcaaaaaccttaacggtgcattcttagcgaataaagttgctaataaaagcattgaaagcgtggaggcaaaagtcgagt

SEQ ID NO: 3 - DNA, 2257 bp, Shewanella baltica
atgacccataacggccaagctgaaacgtcgatagctgacaaggctattgctgacaaaagcgccaagccactgcgtattgcagtgttgcttggcgatgcggtcaaccttgactctcactcagcccaagtattagggacgtttactgaacgtgaaagagttcaaatttgcgctgccgatgccaaccaatcgacagtacataggccgacagctcatgaacccaaagagcatgaaccgtcagtgtatgagcaaaggtcgctcacagccttgttaggccaagcaacaacagccattgagcaaggcaagctcgttgaacttaaatttgaggatggtaatctaccccagtatctgtatttgctcgatggtttacgcgccgccaaactgcgtcttcacgcccacgcgtttattgctggctttgccgctggcaatgaggttacagatgttgcaaatgcggcgactgttgcaaataaggcgttagcggcagcaaagcgcagcccagctcaaaccgttcaacatcagactgtcgccaacacactcaacgaggcatttgttgcgcttcgccaaggcgtaaccgccctcgccgcgcgaacacaagcgccgcttaaaggcactactggtataaaacagacaaacgacaccaatcatcaaactggctattggtttagcgaccaacatcaagcgcgggtgttgtgtctaaatcttgtagcaaagacgtcacatcaagcggatgagtctcgaaatctaagccaaagcctagtgctgacccaaggcacacaactcgccgcgcccaaagcccttgtcgatgaaaaccggctgtttgtgccgataagtagtgacagtattaatgagttaaaagcaaagctgtttcagttgcttagttcactagacattggcgcaccagacacgtcatctgcatcacatcaactcacattctggtttgaacgctacgatgccaacgcaccactggcattagtcttgatggcggcatcaagtgatgatctcaaacttgaagccaaagccatgcttacggcgcttgaaaatgatgctattcgtcatcatggtcaacactttaagacaccagcgggtagctgctttaccgctaagccgctaggggatgcgggactgacttttgtgtatcctggcgttggcacggtttacgccaatatgttcaataacttgcatgagtatttccccgcgctgtatcaccagttagaacgtgaaggcgatttaagcgccatgctgcaatcaccgcagatttatgcggcagatgttaaaaccgcggctggcatgaacctaagtcagcaagcgattagtggcgtgggggccagttatctattcactaaactgttgacccaagtctttaatattaagcctaaaatggcgctcggttactcaatgggcgaagcagccatgtgggccagcctagatgtgtggcaaacaccgcacgctatgattaacgccactgaaaatagcgatattttcaaccatgcgatttctggtgagctaactgctgtgcgccgagcatggcagcttgcggatgatgaagccatagtgtggaacagctttgtggtgcgcgctgatagccatgaaatcaaggcattattgccagagtttcctcgcgcctacttagccatcacccaaggtgatacttgtgtcattgcaggctgcgaaacaagctgtaaagccctgcttgccacgttaggcaaacgcgggattgccgccaatcgcgtcacggcaatgcataccgcgcctgccctgttagtccatgggcaagtacaagatttctatactcaagcgctaaaacctgaggcactggaacctgatgcgctgaaagcggcagcgcaagattcgtctgttcgctttattagcgcagcgcaaactgcgccagtaatcgtggatagccacagcattggccgcgcgattgccgataccttttgttcgccactcgactttagtgcgctcattcaaaatgccactgagcaaggcgcaaggctgtttgtcgaagtgggcgccgataggcaaaccagtacactcatagataaaatcagccatgcccacgcaagccaaagctctgcgaacgcggcgacagccgccattgcctgcaatgccaaaggtgccgacgcgatcaccagcttactcaagtgtttagctcagctaataagccacagagtgccgctttcgctcacgccgcttattcagccattaagtgctaacgcagcccctttatcatcagcagtatcaccaaaaggagaaccccagt

SEQ ID NO: 4 - DNA, 3367 bp, Shewanella baltica
atggcgagtattttgagcgccctgcttcagcttaaaaatcaagggcaattaaaaaaccaagcacagcaacaagctaatcaagtgcagcacgcgctcgttgccacgtttagccaaggtaaatgctcgcagttattgctcagtcaaagtgcgacgcaagcacacagtttgcagcaaaggcttgaacaagacttaacgctttctgagcaaaaacacttaattaaacaagtgacacttggtggccgcgatatctatcagcatatccttgatacgccgttggcggacatagatgcaataaagcaaaaagcccaagccatcactgcattgccaaagcgcagccaacgcaaacatttggcccaaatagcgagcaaagacacgagtggctttgcaacaagcagcccaaccacggctctacaaaaagagacattaagcagcatgccaattaatgccctaagcacgcccaatgacaacgcagctcaaacagagctaaaagacgctgcatttatacgtaatcagcaactcgcccgtgaagcccatttagcctttttacaaagccgtgcgcagggcctgaaactggccgatgccttgatgaaagcccagcttgccagcgagttagccgtcaatggccaagcgacgccggtacaacagcaagccactgttcaggcgccagtgtatgcatctgctcatacacctgagctagctccagttgttaactcagaggcaaacccagctgcgctttatccaaaccatgcaaaagtgcctctgtacacgccgccaacaccgataagcaagccttgcatttgggattatgcggatttagtcgagtacgccgaaggcgatattgctaacgtctttggccaagattacgccattatcgacagctattcgcgccgtgtacgcctgccaacgactgactatttgctggtatctcgggtaacgaaattaaacgcccagatgaaccaatatcagccttgcactatgaccacagaatacgacattcctgtggacgcgccgtatttggtcgatggccaaattccttgggccgtagcggttgaatcgggtcaatgcgacttgatgctgatcagctacttaggtatcgactttgaaaacaagggcgagcgcgtttatcgcctgctcgattgcaccctgaccttccttggcgacttaccgcgcggtggcgacaccctgcgctacgatatttcaatcaatcactttgcccgcaatggcgataccttgttgttcttcttctcctacgaatgtttcgtgggcgacaagctgatcctgaaaatggatggcggctgtgccggcttcttcaccgataaagaactagccgacggcaaaggcgttattcacaccgaagccgaaatcaaagcgcgcaacctcgccttgaacaatccgaataagccgcgctttaatccgttactcaactgcgcgcaaaaccaatttgattacagccaaatccataaactgctcggcgccgatatcggtggctgttttggcggcgcacacgcggcgcatcaagcccaatatggtttgcagccctctttatgttttgcatctgaaaaattcctgatgattgaacaagtcagcaatcttgaggtgcatggcggcgcgtggggcttaggctcagttcaaggccataagcagctcgaagccgatcattggtatttcccgtgtcatttcaagggcgaccaagtgatggcggggtcgttaatggccgaaggctgtggtcaattactgcaattctttatgctacatattggtatgcacctcggtgttaaagatggtcgtttccaaccgctcgaaaacgcgtcacaaaaagtgcgttgtcgcggtcaagtgttgccgcaatcaggcctgctcacctatcgtatggaaatcactgaaatcggtatgagcccgcgcccgtatgctaaggcgaatatcgatattctgctcaatggtaaagtggttgtggacttccaaaaccttggggtgatgatcaaagaagaagccgaatgcacccgctaccttgcggataatgatgccagcacagctgacaatacgactaaaaatgctgccaaaaatgctgcttcggctgtgccgctagtttcgacaacacccgcatcgttcgccgcgccgttgatggcccagctgccagatttaactgcgccaaccaataaaggcgtagtgccgcttaagcatgtgcctgcgccgattgcgcaaacggattcaaagtacgccaaccgcgtgcccgataccctgccgttcacgccgtaccacatgttcgaatttgccacgggcgatatcgaaaactgcttcggccccgatttcagcatctatcgcggccttattccaccgcgcacgccttgcggtgatttacagcttaccacccgcgtcattgcgattgacggcaaacgcggcgagctgaaaaagccttcttcgtgtatcgccgaatacgaagtgcccgcaaacgcttggtattacgataaaaacagtcatcatgctgtgatgccctattcagtgctaatggaaatatcactgcagccaaatggctttatttcaggctatatgggcaccaccttgggcttccccggccaagagctgtttttccgtaacttagacggcagcggtaagctgctgcgccacgtggatttacgcggcaaaaccatagtgaacgactcacgtttgttatcgactgtgattgccggcagcaacatcatccagaatttcagcttcgagttaagctgcgatggcgagcctttctaccaaggtaaagcggtatttggttacttcaagggcgatgcgctgaaaaaccaactcggcatagacaacggcaagatcacacagccttggcatgtgcaaaatggcatagccgccgatagccaaatcaatctgttagataaacagcatcgcagctttaacgcgccagaaggtcagccgcattaccgtttagcgggcggtcagcttaactttatcgacaaggccgacatagtgaaagccggcggtaaagcgggccttggctatttatacgccgagcgcaccattgacccgagtgattggttcttccaattccacttccatcaagatccggtaatgccaggctcattaggggttgaagcgattatcgagctgatgcaaacctatgcgattgaccaagaccttggtgcgggctttaagagtccaaaattcggccagatattatcggatatcaaatggaagtatcgcggccaaatcaacccattaaacaaacagatgtcgctggatgtgcacattaccagcgtgacagacgacaatggcaaacgcatcattatgggcgatgccaacttgagtaaagatggtctgcgaatttatgaagtcaaagatatcgccatctgtattgaagaagctt

SEQ ID NO: 5 - DNA, 1645 bp, Shewanella baltica
atgacaagccatactctcgatcaatttaatagtaataacgaaaaactcagcccttggccgtggcaagtcaacgatgccgcgctgagctttgatatcgactcattaggcaaaaaactcaaagatttaagccaagcctgttacttagtgaatcacagtgaaaaaggcttaggcatagcgcaaacagccgaagtaaccacaagcgacagccaagcgccactaggctcacaccccgtcagcgcctttgcgcccgcccttggcacccaaagtttaggcgacagtaattttcgccgcgtacacggggttaaatacgcttactacgctggcgctatggctaacggtattgcctcagaagaactggttatcgcgctgggccaagcgggcattttgtgttcgtttggcgcggcggggttaatcccatcccgcgttgaagcggccattactcgcattcaagcggcgctgcctaatggtccttacgcctttaatttaattcacagcccaagcgagcccgcattagagcgcggcagtgttgagttgttcttaaaacataaagtgcgcacggtcgaagcctcggcatttttaggtttaacgccacaaatcgtctattaccgcgcagcaggtttgagccgcgacgcacatggcgacatcgtcattggcaacaaagtcatagccaaaatcagtcgcaccgaagtcgcgactaagtttatggagccggcgcctgccaaaattctgcagcaattagtcagtgaaggccttatcagccaagatcaaatggcgatggcgcaacttgtacccatggcggacgatatcacggccgaagccgattctggcggccataccgacaatcgtccactggtcacgctattgccgacgattttggcgctcaaagatgaaatccaagctaagtatcaatacaagacgcccatccgtgtgggagcaggcggcggcgttggtacccccgacgcagcattagccaccttcaacatgggcgcggcctttatcgtcacaggttcaatcaaccaagcgtgtgtggaggcgggcgcgagcgaacacacacgtaagttactcgccaccacagaaatggccgatgtgactatggcacccgccgccgatatgttcgaaatgggcgtgaaattacaagtggttaagcgtggcactctgttcccgatgcgcgccaataagctttatgaaatctacacccgttacgattcaattgatgccatccccgcggacgagcgtaaaaagctcgaagagcaagtgtttcgcgcatcactcgatgacatttgggcgggcactgtcgcccactttaacgagcgcgatcctaagcaaatcgagcgcgcactggataaccccaaacgtaagatggcgttgattttccgctggtatttgggtctgtctagccgctggtcgaacacaggtgaagtcggccgcgaaatggattaccaaatttgggcaggccctgcccttggcgcatttaatgcgtgggcaaaaggcagttatttagatgactataaagcccgtaatgcggtcgatttagccaaacatttaatggtgggcgcggcctatcaatcccggattaacttgctgttatcccaaggggttagcattccagttagcctgcaacgctggaaaccgctaaatcgttttt

SEQ ID NO: 6 - DNA, 1004 bp, Shewanella baltica
aataatcagcaagtaagctctctatgctgctcaactgcgcggttagcgttatgggaagcgcggcattaccggcatgcgctaagctaatggcaaagcgatactcctcggtcaaacgaccaaaatgcgtttgccaatgcaaggagtcagctaaatccagtgtgtgtattgggatttgtagcgggacttgtaatggaatatgtggataaacattaagcattgaagctacgccgcaggcttgcctcaatccggcatcttggcatgataactcaagcgattctgccgccaattgctcaaacggcttaagctcaaaggcaaaggatttgagcgactgcgctaaacctaaaccagtcgccttaatataggactcctttagcgcccataaatcgaaaaagcgctgtcgctgactcgcttcatcggcgagagcgagcaaggctgcagtttcttggggagaaaaatagtgatttaggattggatagatatccgtcttcggccgcgacctttcaatatccaccccaaataaaccagactcaacgccatcaaactgagccacaccaatcaataaccaatcgccgctatggcttaggttaaactcgagaccagtttgctgccctaatgtcgccgttaaactcggtttgcccttagcgccatattcaaaacaccactcatgtggcgataaatcagcatagcgagataacacggtccgtagcgccgccctcacctgtaacccctttatttgcaccttgggatcgcgataacgcctgacctttgcgagttcatcatcacttaaccaagactcggcaagtgatgtttgcgccgctgaaatatcgttcagtggaataaaaaatagatccacattcacgccgtttttgctagatgttagagagtcggtcattcgagagcctagctgtacaaagccctgcggctgatgagttatcattggctggctttttggagagcagttagcaacattcttatctggttcattattgtctaggttcatcatt

(19) United States
(12) Patent Application Publication
(10) Pub. No.: US 2017/0209501 A1
(43) Pub. Date: Jul. 27, 2017

(54) PRODUCTION OF OMEGA 3 FATTY ACIDS BY RECOMBINANT ESCHERICHIA COLI NISSLE 1917

(71) Applicant: Micro-Omegapro Inc., Guelph (CA)

(72) Inventors: Mitra Amiri-Jami, Guelph (CA); Mansel W. Griffiths, Rockwood (CA)

(21) Appl. No.: 15/408,870

(22) Filed: Jan. 18, 2017

Related U.S. Application Data
(60) Provisional application No. 62/281,865, filed on Jan. 22, 2016.

(51) Int. Cl. A61K 35/74; C12N 15/52; A61K 31/202; A61K 45/06; A61K 9/00; C12N 15/70; C12N 9/10

(52) U.S. Cl. CPC A61K 35/74; C12N 15/70; C12N 15/52; C12N 9/1029; C12Y 203/01094; A61K 45/06; A61K 9/0053; A61K 31/202

(57) ABSTRACT

Described are recombinant Escherichia coli Nissle 1917 (“EcN”) cells transformed with genes that express proteins or polypeptides involved in omega 3 fatty acid biosynthesis. The recombinant EcN cells produce eicosapentaenoic acid (EPA) and/or docosahexaenoic acids (DHA). Associated compositions, methods and uses of the recombinant EcN cells are also provided.

### RELATED APPLICATIONS

This application claims priority to U.S. Provisional Application No. 62/281,865 filed Jan. 22, 2016, the entire contents of which are hereby incorporated by reference.

### INCORPORATION OF SEQUENCE LISTING

A computer readable form of the Sequence Listing “25674-P49600US01_SequenceListing.txt” (53,248 bytes), submitted via EFS-WEB and created on Jan. 18, 2017, is herein incorporated by reference.

### FIELD

The present disclosure relates to the production of omega 3 fatty acids and more specifically to recombinant Escherichia coli Nissle 1917 for the production of eicosapentaenoic acid (EPA) and/or docosahexaenoic acid (DHA).

### BACKGROUND

Eicosapentaenoic acid (EPA, 20:5n3) and docosahexaenoic acid (DHA, 22:6n3) are important omega 3 fatty acids (ω3FAs). In the past decade, EPA and DHA have been promoted as essential dietary components due to their wide ranging physiological effects and their impact on human health. Numerous studies have shown the involvement of EPA and DHA in fetal development (Ramakrishnan et al., 2010), immune function (Yaqoob & Calder, 2007), prevention of Alzheimer's disease (Tully et al., 2003), and protection against cardiovascular disease (Kris-Etherton et al., 2002; Swanson et al., 2012). Omega 3 fatty acids are known as essential fats and currently, fish and fish oil are the main dietary sources for EPA and DHA (Adarme-Vega et al., 2014). The increased awareness of the health benefits of EPA and DHA has led to greater use of fish oils in the pharmaceutical, nutraceutical and agricultural sectors. A higher demand for EPA and DHA will increase the strain on fish populations, which are already in decline, therefore recent investigations have focused on microorganisms as alternative sources of EPA/DHA.

Bacterial biosynthesis of EPA and DHA is limited to a small number of gram-negative marine bacteria such as Shewanella and Colwellia (Bowman et al., 1998). However, these marine bacteria cannot be used for EPA/DHA production due to difficulty in culturing them on an industrial scale. Currently, there is no recombinant source of EPA/DHA available for animal and human consumption. Therefore, in the last decade research has focused on the production of EPA/DHA by recombinant microorganisms such as E. coli, cyanobacteria or by plants (Abbadi et al. 2004; Lopez et al. 2013; Orikasa et al. 2004; Orikasa et al. 2009; Takeyama et al. 1997; Yu et al. 2000). Recently, a gene cluster has been isolated from a marine bacterium, Shewanella baltica MAC1, and cloned in laboratory E. coli strains and Lactococcus lactis subsp. cremoris MG1363 (Amiri-Jami & Griffiths 2010; Amiri-Jami et al., 2014). Sixteen genes were isolated from Shewanella baltica MAC1 of which five (pfaA, pfaB, pfaC, pfaD and pfaE) were shown to be responsible for both EPA and DHA production (Amiri-Jami & Griffiths 2010). Transformation of this gene cluster to different strains of E. coli and Lactococcus lactis MG1363 resulted in production of both EPA and DHA by these bacteria (Amiri-Jami & Griffiths 2010) and production was high in recombinant E. coli strains (Amiri-Jami et al., 2014). However, these strains may not be suitable for commercial production of EPA/DHA.

Escherichia coli Nissle 1917 (EcN) is one of the most studied probiotic bacteria. Probiotic bacteria are living microorganisms that are non-pathogenic and have beneficial effects on human health by improving the microbial balance of the indigenous microbiota (Troge et al., 2012). Several studies have shown that probiotics modulate immune responses (Oelschlaeger, 2010), act as an intestinal barrier (Ohland & Macnaughton, 2010), have an anti-inflammatory potential (Helwig et al., 2006), and are able to secrete bioactive molecules which down-regulate virulence gene expression of bacterial pathogens (Medellin-Pena et al., 2007; Bayoumi & Griffiths, 2012). Probiotic EcN 1917 was isolated by Alfred Nissle in 1917 during the First World War (Nissle, 1918). It is reported that EcN has anti-invasive effects against enteroinvasive bacterial pathogens through a secreted component (microcins) that does not rely on direct physical contact with the invading bacteria or host epithelial cells (Altenhoefer et al., 2004). Moreover, EcN has strong colonization properties and acts as a safe carrier for targeted delivery of recombinant proteins into the host intestinal mucosa (Westendorf et al., 2005). In addition, EcN 1917 has been used for several decades as a probiotic, affording protection against a variety of intestinal disorders such as inflammatory bowel disease (Hernando-Harder et al., 2008).

### SUMMARY

Omega 3 fatty acids, including eicosapentaenoic acid (EPA) and docosahexaenoic acid (DHA), have been reported to have beneficial effects on human health. However, there are relatively few sources of EPA and/or DHA suitable for commercial production. There is remains a need for novel sources of omega 3 fatty acids including those suitable for use in humans.

The naturally occurring probiotic bacterium Escherichia coli Nissle 1917 is unable to produce EPA or DHA. The inventors have surprisingly determined that E. coli Nissle 1917 transformed with a plasmid carrying a EPA/DHA gene cluster isolated from the marine bacterium Shewanella baltica MAC1comprising the genes pfaA, pfaB, pfaC, pfaD and pfaE is capable of producing significant quantities of omega 3 fatty acids. More specifically, as shown in the Examples transgenic E. coli Nissle produced EPA when grown at 10° C. (16.52±1.4 mg g⁻¹ cell dry weight), 15° C. (31.36±0.25 mg g⁻¹ cell dry weight), 20° C. (13.71±2.8 mg g⁻¹ cell dry weight), 25° C. (11.33±0.44 mg g⁻¹ cell dry weight) or 30° C. (0.668±0.073 mg g⁻¹ cell dry weight). Transcriptomic analysis using Reverse Transcription qPCR showed up-regulation of the entire gene cluster in E. coli Nissle. Among EPA/DHA genes, pfaB, pfaC and pfaD were over-expressed (expression ratios of 181.9, 39.86 and 131.61, respectively) as compared to pfaA (expression ratio of 3.40) and pfaE (expression ratio of 4.05). The EPA/DHA-producing probiotic E. coli Nissle may therefore be useful as a source for the commercial production of EPA and/or DHA.

In one embodiment, the recombinant EcN cells described herein may be cultured in bioreactors and EPA/DHA extracted from the culture in order to produce an alternative and safe source for EPA/DHA. Currently, many omega-3 supplements are sourced from fish, which may be retain an unpleasant smell and/or taste or contain lead or mercury contamination. In one embodiment, the EPA/DHA produced using the EcN cells and methods described herein is free of unpleasant smells and/or tastes associated with fish products and is free of lead or mercury contamination. The EPA/DHA produced using the EcN cells and methods described herein may be used in the production of pharmaceutical, nutraceutical, cosmetic and/or agricultural products that contain omega-3 fatty acids.

Accordingly, in one embodiment there is provided a recombinant Escherichia coli Nissle 1917 (EcN) cell or a variant thereof, comprising one or more genes selected from pfaA, pfaB, pfaC, pfaD and pfaE, wherein the cell produces one or more omega 3 fatty acids. In one embodiment, the cell comprises the genes pfaA, pfaB, pfaC, pfaD and pfaE. In one embodiment, the cell comprises genes having sequence identity, such as at least 80%, 90%, 95% or 99% sequence identity to SEQ ID NO: 2 (pfaA), SEQ ID NO: 3 (pfaB), SEQ ID NO: 4 (pfaC), SEQ ID NO: 5 (pfaD) and/or SEQ ID NO: 6 (pfaE). In one embodiment, the cell produces eicosapentaenoic acid (EPA) and/or docosahexaenoic acid (DHA).

In one embodiment, the cell is transformed with a nucleic acid molecule comprising a gene cluster comprising pfaA, pfaB, pfaC, pfaD and pfaE. In one embodiment, the cell comprises a nucleic acid molecule comprising a gene cluster comprising genes having sequence identity, such as at least 80%, 90%, 95% or 99% sequence identity to SEQ ID NO: 2 (pfaA), SEQ ID NO: 3 (pfaB), SEQ ID NO: 4 (pfaC), SEQ ID NO: 5 (pfaD) and SEQ ID NO: 6 (pfaE). In one embodiment, the nucleic acid molecule in a vector. Optionally, the cell is transformed with two or more nucleic acid molecules comprising the same or different genes selected from pfaA, pfaB, pfaC, pfaD and pfaE. In one embodiment, the cell comprises a gene cluster with at least 80%, 90%, 95% or 100% sequence identity to SEQ ID NO: 1, wherein the gene cluster comprises pfaA, pfaB, pfaC, pfaD and pfaE.

In one embodiment, the recombinant cell is a recombinant Escherichia coli Nissle 1917 (EcN) cell or a variant thereof. For example, in one embodiment, the EcN cell comprises a genome with at least 80%, 90%, 95%, 97%, 98%, 99% or 100% sequence identity to the EcN genome deposited under GenBank under accession no. CAPM00000000. EcN cells suitable for transformation with a nucleic acid molecule encoding the pfaA, pfaB, pfaC, pfaD and/or pfaE genes described herein include DSM 6601 in the German Collection for Microorganisms in Braunschweig, Germany or the probiotic E. coli Nissle bacteria available commercially as Mutaflor™.

The recombinant EcN cells described herein have been demonstrated to produce the omega 3 fatty acids EPA and DHA. For example, in one embodiment, the recombinant cells described herein produce more EPA relative to wild type EcN cells or relative to wild type S. baltica MAC1 cells. In one embodiment, the recombinant EcN cells described herein produce more EPA relative to wild type S. baltica MAC1 cells when cultured at 15° C.

In one embodiment, the recombinant EcN cells exhibit increased gene expression of the pfaA, pfaB, pfaC, pfaD and/or pfaE genes relative to wild type S. baltica MAC1. In one embodiment, the recombinant EcN cell comprises pfaB and expresses a higher level of pfaB relative to the level of pfaB expressed by wild type S. baltica MAC1 when cultured at 15° C.

In one embodiment, the recombinant EcN cells described herein produce omega 3 fatty acids. In one embodiment, the EcN cells produce at least 5, 10, 15, 20, 25 or 30 mg of eicosapentaenoic acid (EPA) per gram of cell dry weight (g⁻¹ of CDW).

In another aspect, there is provided a composition comprising the recombinant EcN cells described herein and a pharmaceutically acceptable carrier. In one embodiment, there is provided a composition comprising the recombinant EcN cells described herein and a culture media. Optionally, the EcN cells are freeze-dried or lyophilized either alone or in a composition. In one embodiment, the composition comprises one or more additional bacterial cells, optionally one or more probiotic bacterial cells or prebiotic chemicals.

In one aspect, there is provided the use of the recombinant cells and/or compositions as described herein as a probiotic and/or nutritional supplement in a subject in need thereof.

Also provided is a method for producing one or more omega 3 fatty acids in vivo comprising administering to the subject the recombinant EcN cells and/or a composition comprising the recombinant EcN cells as described herein. In one embodiment, there is provided a method for producing one or more omega 3 fatty acids in the gastrointestinal tract of a subject. In one embodiment, the omega 3 fatty acid is EPA and/or DHA. In one embodiment, the method comprises orally administering to the subject the recombinant cell or composition.

In another aspect, there is provided a method for the production of omega 3 fatty acids using the recombinant EcN cells described herein. In one embodiment, the omega 3 fatty acid is EPA and/or DHA. In one embodiment, the method comprises culturing one or more recombinant EcN cells of under conditions suitable for the production of omega 3 fatty acids. For example, in one embodiment, the recombinant EcN cells are cultured at a temperature between 5° C. and 30° C. In one embodiment, the recombinant EcN cells are cultured at a temperature between 10° C. and 25° C., between 10° C. and 20° C., between 13° C. and 17° C., or optionally about 15° C. In one embodiment, the method further comprises isolating one or more omega 3 unsaturated fatty acids from the cell culture.

In another aspect, there is provided an isolated nucleic acid molecule comprising a gene cluster comprising pfaA, pfaB, pfaC, pfaD and pfaE. In one embodiment, the nucleic acid molecule is a vector. In one embodiment, the nucleic acid molecule has at least 80%, 85%, 90%, 95%, 96%, 97%, 98%, 99% or 100% sequence identity to SEQ ID NO: 1.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows EPA production of gEcN harboring pfBS-PS (pCC1FOS+20 kbp EPA/DHA cluster). Genetically modified E. coli Nissle carrying the 20 kbp gene cluster was cultured in LB broth and grown at 15° C. Total fatty acid was extracted from cells and analyzed as described in the Examples. The data shown are the result of triplicate samples.

FIG. 2 shows a comparison of EPA production by gEcN and S. baltica MAC1 at different temperatures. The gEcN clone was grown at 10° C., 15° C., 20° C., 25° C., 30° C. in triplicates. Total fatty acids was extracted from freeze-dried cells and analyzed by GC as described in the Examples. The data shown are the means±the standard deviations of the result of three independent experiments.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

As used in the present disclosure, the singular forms “a”, “an” and “the” include plural references unless the content clearly dictates otherwise. For example, an embodiment including “a recombinant cell” should be understood to present certain aspects with multiple recombinant cells.

The term “and/or” as used herein means that the listed items are present, or used, individually or in combination. In effect, this term means that “at least one of” or “one or more” of the listed items is used or present.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “omega 3 fatty acids” as used herein refers to polyunsaturated fatty acids with a double bond at the third carbon atom from the end of the carbon chain. The term “unsaturated fatty acids” as used herein refers to carboxylic acid compounds containing long aliphatic tail chains in which there is at least one double bond.

The term “DHA” as used herein refers to docosahexaenoic acid, an omega 3 fatty acid. DHA is a carboxylic acid characterized by a chemical structure having a 22-carbon chain containing six cis double bonds.

The term “EPA” as used herein refers to eicosapentaenoic acid, an omega 3 fatty acid. EPA is a carboxylic acid characterized by a chemical structure having a 20-carbon chain containing five cis double bonds.

The term “vector” as used herein refers to a nucleic acid molecule that can be used to transfer genetic material into a host cell where it can be replicated and/or expressed. Optionally, the vector includes an origin of replication, a multicloning site and/or insert, and a selectable marker. For example, in one embodiment the vector may contain an insert comprising one or more genes selected from pfaA, pfaB, pfaC, pfaD and/or pfaE. In one embodiment, the vector contains a gene cluster comprising pfaA, pfaB, pfaC, pfaD and pfaE. Examples of vectors include, but are not limited to, plasmids, bacteriophage, modified viruses (e.g., replication defective retroviruses, adenoviruses or adeno-associated viruses), cosmids, and artificial chromosomes such as bacterial artificial chromosomes (BACs), so long as the vector is compatible with EcN.

The terms “Escherichia coli Nissle 1917” or “EcN” as used herein refer to a non-pathogenic gram-negative probiotic bacteria Escherichia coli strain that is capable of colonizing the human gut. In one embodiment, the EcN cells are of serotype O6:K5:H1. Examples of Escherichia coli Nissle 1917 bacteria include those available as DSM 6601 from the German Collection for Microorganisms in Braunschweig, Germany or commercially as the active component in Mutaflor® (Ardeypharm GmbH, Herdecke, Germany). EcN cells suitable for transformation with a nucleic acid molecule encoding the pfaA, pfaB, pfaC, pfaD and/or pfaE genes are also available from the Canadian Research Institute for Food Safety Culture Collection. Genetic variations of Escherichia coli Nissle 1917 are contemplated in the present disclosure. Accordingly, in one embodiment the recombinant EcN cell comprises a genome with at least 80%, 90%, 95%, 97%, 98%, 99%, 99.5% or 100% sequence identity to the EcN genome deposited under GenBank under accession no. CAPM00000000. EcN cells may also be identified using the Riboprinter™ microbial characterization system from DuPont. In one embodiment, the EcN cells exhibit at least a 85%, 90%, or 95% match with database entries for EcN cells based on genetic fingerprints.

### Nucleic Acid Molecules and Recombinant Cells

In one aspect, there is provided an isolated nucleic acid molecule comprising a gene cluster comprising pfaA, pfaB, pfaC, pfaD and pfaE. In one embodiment, the gene cluster is isolated from S. Baltica (MAC1). In one embodiment, the nucleic acid molecule has at least 80%, 85%, 90%, 95%, 96%, 97%, 98%, 99% or 100% sequence identity to SEQ ID NO: 1. In one embodiment, the nucleic acid molecule comprises or consists of the nucleic acid sequence of SEQ ID NO: 1 In one embodiment, the nucleic acid molecule is a vector. In one embodiment, the nucleic acid molecule is a vector comprising an insert comprising or consisting of SEQ ID NO: 1. In one embodiment, transformation of an EcN cell with the vector results in the production of omega 3 unsaturated fatty acids.

In one aspect, the present disclosure is directed towards a recombinant Escherichia coli Nissle 1917 (EcN) cell, or a variant thereof, transformed with a nucleic acid molecule containing one or more genes involved in the biosynthesis of omega 3 fatty acids. As demonstrated in the Examples, the inventors have determined that EcN cells transformed with pfaA, pfaB, pfaC, pfaD and pfaE are useful for the recombinant production of omega 3 fatty acids and in particular EPA.

Accordingly, in one embodiment there provided a recombinant EcN cell or variant thereof comprising pfaA (SEQ ID NO: 2), pfaB (SEQ ID NO: 3), pfaC (SEQ ID NO: 4), pfaD (SEQ ID NO: 5) and pfaE (SEQ ID NO: 6).

Optionally, the recombinant EcN cell comprises one or more genes with sequence identity to pfaA (SEQ ID NO: 2), pfaB (SEQ ID NO: 3), pfaC (SEQ ID NO: 4), pfaD (SEQ ID NO: 5) and/or pfaE (SEQ ID NO: 6). For example, in one embodiment, the cell comprises one or more nucleic acid sequences with at least 70%, 75%, 80%, 85%, 90%, 95%, 96%, 97%, 98%, 99% or 100% sequence identity to one or more of SEQ ID NO: 2, SEQ ID NO: 3, SEQ ID NO: 4, SEQ ID NO: 5 and SEQ ID NO: 6.

In one embodiment, the recombinant EcN cell comprises a sequence comprising a gene cluster that includes pfaA, pfaB, pfaC, pfaD and pfaE. As used herein, the term “gene cluster” refers to a continuous linear nucleic acid sequence comprising two or more genes. For example, in one embodiment the recombinant EcN cell comprises a nucleic acid with sequence identity to the gene cluster shown in SEQ ID NO: 1. In one embodiment, the recombinant EcN cell comprises a gene cluster with at least 80%, 85%, 90%, 95%, 96%, 97%, 98%, 99% or 100% sequence identity to SEQ ID NO:1. In one embodiment, the cell comprises a plurality of nucleic acids each encoding for a gene cluster that includes pfaA, pfaB, pfaC, pfaD and pfaE.

Sequence identity can be determined according to sequence alignment methods known in the art. Examples of these methods include computational methods such as those that make use of the BLAST algorithm, available online from the National Center for Biotechnology Information. Sequence identity is most preferably assessed by the algorithm of BLAST version 2.1 advanced search. BLAST is a series of programs that are available, for example, online from the National Institutes of Health. References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_141; Altschul, S. F., Madden, T. L., Schaffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

Percent sequence identity or homology between two sequences is determined by comparing a position in the first sequence with a corresponding position in the second sequence. When the compared positions are occupied by the same nucleotide or amino acid, as the case may be, the two sequences are conserved at that position. The degree of conservation between two sequences is often expressed as a percentage representing the ratio of the number of matching positions in the two sequences to the total number of positions compared.

The genes, gene clusters or variants thereof described herein involved in the biosynthesis of omega 3 fatty acids may be cloned into a vector, transformed into EcN cells and expressed in order to produce omega 3 fatty acids. Techniques for cloning nucleic acid molecules and the transformation and expression of vectors in bacterial cells such as EcN are known in the art. Examples of such techniques are described in Sambrook et al. Molecular Cloning: A Laboratory Manual, 4th edition. Cold Spring Harbor Laboratory Press, 2013, incorporated herein by reference. Nucleic acid molecules containing genes coding for one or more of pfaA, pfaB, pfaC, pfaD and pfaE may be cloned into one or more suitable expression vectors. Suitable expression vectors include, but are not limited to cosmids, plasmids, modified viruses (e.g., replication defective retroviruses, adenoviruses and adeno-associated viruses) or artificial chromosomes, so long as the vector is compatible with EcN.

In one embodiment, the vector may also contain a selectable marker gene that facilitates the selection of host cells transformed or transfected with a recombinant molecule of the disclosure. Examples of selectable marker genes are genes encoding a protein which confers resistance to certain drugs, such as G418 and hygromycin.

As used herein, the term “transformation” refers to a process for introducing exogenous nucleic acids into a bacterial cell. The terms “transformed,” “transgenic,” and “recombinant” refer to a host organism such as a bacterial cell, into which an exogenous nucleic acid molecule such as a plasmid or other vector has been introduced. In one embodiment, the recombinant EcN cells described herein may contain multiple copies of the pfaA, pfaB, pfaC, pfaD and/or pfaE genes or associated gene cluster. In one embodiment, the pfaA, pfaB, pfaC, pfaD and/or pfaE genes or associated gene cluster are present in the EcN cell in a copy number greater than 1 such that, on average, each cell contains at least one recombinant vector. In one embodiment, the recombinant EcN cell comprises a recombinant vector comprising multiple copies of each of the one or more genes associated with omega 3 fatty acid biosynthesis.

As used herein, the term “exogenous” refers to a nucleic acid molecule (for example, a circular plasmid DNA sequence), gene or protein that originates from a source foreign to the particular bacterial cell into which it is introduced. An exogenous nucleic acid molecule may be introduced into a bacterial cell either in a stable or transient fashion, in order to produce one or more RNA molecules and/or one or more polypeptide molecules. For example, the genes pfaA, pfaB, pfaC, pfaD and pfaE from S. baltica MAC1 described herein are exogenous when introduced into EcN cells.

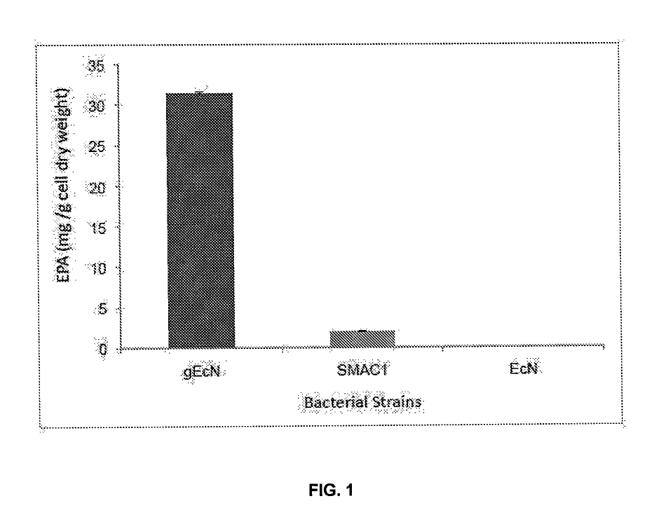

The recombinant EcN cells described herein are readily distinguished from wide type EcN cells. In one embodiment, recombinant EcN cells produce detectable levels of omega 3 fatty acids, such as EPA and/or DHA. As shown in FIG. 1, recombinant EcN cells (gEcN) produce significantly more EPA than S. baltica MAC1 cells (SMAC1) or EcN control cells (EcN). Wild type S. baltica cells are available, for example, from the Canadian Research Institute for Food Safety Culture Collection.

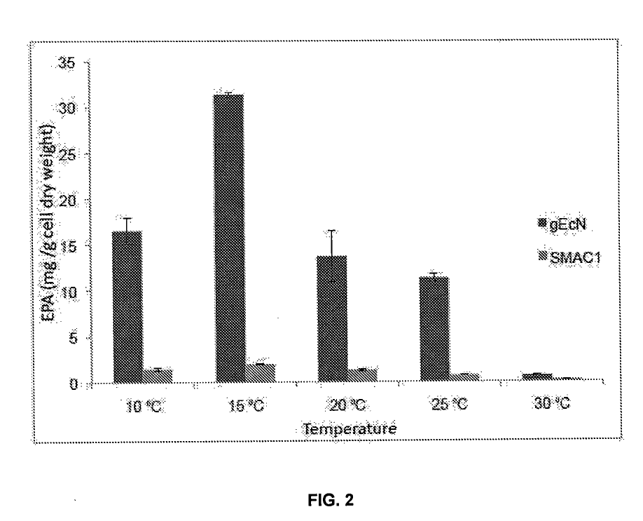

As shown in FIG. 2 the inventors have also determined that recombinant EcN cells produce higher levels of omega 3 fatty acids relative to wild type S. baltica MAC1 cells when cultured at a variety of temperatures. For example, recombinant EcN cells produce more EPA relative to S. baltica MAC1 cells when cultured at 10° C., 15° C., 20° C., 25° C. or 30° C. In one embodiment, the recombinant EcN cells described herein produce at least 5, 10, 15, 20, 25 or 30 mg of eicosapentaenoic acid (EPA) per gram of cell dry weight (g⁻¹ of CDW).

### Compositions

The present disclosure also provides compositions comprising the recombinant EcN cells as described herein that produce one or more omega 3 fatty acids.

In one embodiment, the compositions comprise recombinant EcN cells and a carrier, optionally a pharmaceutically acceptable carrier. Pharmaceutically acceptable carriers may be chosen to permit oral administration or administration by any other known route. Suitable carriers are described, for example, in Remington's Pharmaceutical Sciences (2003—20th edition) and in The United States Pharmacopeia: The National Formulary (USP 24 NF19) published in 1999. In one embodiment, the compositions described herein include recombinant EcN cells and an animal feed suitable for administration to animals such as poultry.

On this basis, the compositions include, albeit not exclusively, compositions containing the recombinant EcN cells in association with one or more acceptable vehicles, carriers or diluents. These compositions may be supplied, without limitation, as powders, caplets or tablets whereby the bacterial cells are in a dormant but alive state achieved through the use of lyophilization or freeze-drying. Other components that may be present in such compositions include water, surfactants (such as Tween), alcohols, polyols, glycerin and vegetable oils, for example. The lyophilized powder may be reconstituted with sterile water or another vehicle, carrier or diluent prior to administration to the patient. In one embodiment, the composition comprises recombinant EcN cells in association with animal feed, such as an animal feed suitable for farm animals. In one embodiment, the animal feed is for poultry.

Suitable pharmaceutically acceptable carriers include essentially chemically inert and nontoxic compositions that do not interfere with the effectiveness of the biological activity of the composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. In one embodiment, the compositions described herein contain a therapeutically effective amount of recombinant EcN cells that produce omega 3 fatty acids, together with a suitable amount of carrier.

Optionally, the composition may comprise one or more additional probiotic microorganisms such as Lactobacillus or Bifidobacterium. Alternatively or in addition, the composition may comprise one or more prebiotic chemicals. As used herein, the term “prebiotic chemicals” refers to a substance that stimulates the growth of beneficial bacteria such as probiotic bacteria. Prebiotics include, but are not limited to, fermentable carbohydrates such as oligosaccharides, galactans and beta-glucans.

Also provided are cell cultures comprising populations of recombinant EcN cells as described herein. For example, in one embodiment there is provided a cell culture comprising recombinant EcN cells that produce omega 3 fatty acids and culture media. In one embodiment, the culture media contains glucose. In one embodiment, the culture media is LB media or another culture media known in the art to support the growth of EcN cells.

### Uses and Methods

In one aspect, the present disclosure also relates to uses of the recombinant EcN cells and/or compositions as described herein.

In one embodiment, the recombinant EcN cells and compositions disclosed herein may be used as a probiotic. As used herein, “probiotic” refers to live, non-pathogenic microorganisms that beneficially affect their animal or human hosts. The administration of probiotics may assist in maintaining the natural balance of microflora in the intestines or may improve the properties of native microflora, or have other beneficial effects such as for the prevention or treatment of disease.

In another embodiment, the recombinant EcN cells and compositions disclosed herein may be used as a nutritional supplement. As used herein, “nutritional supplement” refers to a dietary addition intended to provide nutrients that may otherwise not be consumed in sufficient quantities, such as omega 3 fatty acids. In one embodiment, the recombinant EcN cells and compositions disclosed herein may be used as a nutritional supplement to increase the production of omega 3 fatty acids by an animal, such poultry.

The recombinant EcN cells and compositions disclosed herein may be used in vivo, such as in a subject in need thereof. In one embodiment, the EcN cells and compositions are for oral use. In one embodiment, the subject is a human. In one embodiment, the subject is an animal, such as a mammal. In one embodiment, the subject is a farm animal. In one embodiment, the farm animal is poultry. In one embodiment, the animal is a chicken.

In another embodiment, there is provided a method for producing omega 3 fatty acids in the gastrointestinal tract of a subject comprising administering to the subject a recombinant EcN cell or composition thereof as described herein. In one embodiment, the recombinant EcN cells or composition thereof is orally administered to the subject.

The recombinant EcN cells disclosed herein may be used for the production of omega 3 unsaturated fatty acids such as EPA and/or DHA. In one embodiment, the recombinant EcN cells may be used for the production of EPA.

In one aspect, the present disclosure provides methods for the production of omega 3 fatty acids in vitro. In one embodiment, there is provided a method for the production of an omega 3 fatty acid comprising culturing one or more recombinant EcN cells under conditions suitable for the production of omega 3 unsaturated fatty acids by the cells. As shown in FIG. 2, recombinant EcN cells produce EPA when cultured at a variety of different temperatures. Remarkably, the production of EPA has also been demonstrated to increase at around 15° C. relative to culturing the cells at 10° C. or 30° C.

Accordingly, in one embodiment, the methods described herein include culturing the recombinant EcN cells at a temperature between 5° C. and 30° C., between 10° C. and 25° C. or between 10° C. and 20° C. in order to produce omega 3 fatty acids. In a preferred embodiment, the methods described herein include culturing the recombinant EcN cells at a temperature between 13° C. and 17° C., optionally about 15° C.

The terms “produce” or “production” as used herein refers both to small-scale production and large-scale production of omega 3 unsaturated fatty acids such as EPA and DHA. Both small- and large-scale production may make use of bioreactors or incubators where the recombinant microorganisms are grown to a specific density in a first phase and the production of omega 3 fatty acids occurs in a second phase. Various culture conditions and culture media, such as Luria Bertani (LB) broth, are known in the art to allow for small- and large-scale fermentation of E. coli cultures such as EcN.

In one embodiment, the methods described herein include isolating or separating one or more omega 3 unsaturated fatty acids from the culture. Suitable isolation and separation techniques are well known in the art. For example, the harvested biomass may be centrifuged and dried (e.g. by freeze drying overnight, spray drying, tunnel drying, vacuum drying or other similar methods) and the fatty acids extracted thereafter (e.g. as described previously in Amiri-Jami & Griffiths, 2010).

The omega 3 unsaturated fatty acids produced using the materials and methods described herein may be used in a variety of applications. For example, in one embodiment, the omega 3 unsaturated fatty acids are for use in a nutritional supplement. In one embodiment, the omega 3 unsaturated fatty acids are for use in a cosmetic and/or skin care product. In one embodiment, the omega 3 unsaturated fatty acids produced using the methods described herein are useful for topical application to the skin and/or hair of a subject to improve the look or feel of the skin and/or hair of the subject. In one embodiment, the omega 3 unsaturated fatty acids are for use in an agricultural supplement or animal feed.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### EXAMPLES

### Example 1

### Cloning and Characterization of Recombinant EcN Cells for the Production of Omega 3 Fatty Acids

### Materials and Methods

Strains, plasmids and growth conditions: The bacterial strains and plasmids used in this Example are described in Table 1. EcN 1917 cells obtained from the culture collection of the Canadian Research Institute for Food Safety (CRIFS), University of Guelph, were purified several times by sub-culturing an isolated colony on fresh Luria Bertani (LB) Agar (Difco, Detroit, USA) incubated at 37° C. overnight. An isolated colony of transformant E. coli EPI300T1 was inoculated into a 50 mL sterile tube containing 10 mL LB broth supplemented with 12.5 μg mL-1 chloramphenicol (LBCm) and subsequently incubated at 37° C. overnight while shaking at 300 r.p.m for plasmid isolation. A single colony of purified EcN harboring pfBS-PS, EcN (as negative controls) and S. baltica MAC1 (as positive control) were transferred to 10 mL of LB broth supplemented with 12.5 μg mL-1 chloramphenicol (LBCm), LB broth and Marine broth (MB), respectively and were incubated at 37° C. (E. coli strains) and 30° C. (S. baltica MAC1) overnight. Sterile 250 mL flasks containing 200 mL LBCm, LB and MB were inoculated with 2 mL of each overnight culture and incubated at 10° C. for 8 days, 15° C. for 4 days, 20° C. for 2 days, 25° C. for 1 day or 30° C. for 1 day for fatty acid extraction.

Transformation of E. coli Nissle: E. coli Nissle was transformed with the pfBS-PS plasmid carrying the EPA/DHA gene cluster, previously isolated from a marine bacterium. Plasmid pfBS-PS was isolated from 3 mL overnight E. coli EPI300T1 culture using a QIAprep Spin Miniprep Kit (Qiagen,Toronto, Canada) as described by the manufacturer. The isolated pfBS-PS plasmid was digested with NotI-HF to confirm that the plasmid contained the large 20 kbp EPA/DHA gene cluster. EcN 1917 was transformed with pfBS-PS by electroporation using the GenePulser Xcell Electroporation System (Bio-Rad Laboratories, Mississauga, ON). For electroporation, the pfBS-PS was added to 50 μL of the ice-cold EcN cells and then transferred to an ice-cold 0.2 cM electroporation cuvette. A single pulse was applied at field strength of 2.5 kV, 200Ω resistance and 25 μF capacitance. Immediately, after electroporation, the cell suspension was mixed with 0.95 mL of S.O.C. medium and incubated at 37° C. for 1 h with shaking at 250 r.p.m. Appropriate dilutions of the cells were spread on LB plates containing 12.5 μg mL-1 chloramphenicol. Antibiotic resistant colonies were selected after 18 h incubation at 37° C. To confirm if selected EcN transformants contained the 20 kbp EPA/DHA gene cluster, colony PCR for pfaA and pfaD genes was performed using primers 5A and 8D as described previously (Amiri-Jami & Griffiths, 2010). Isolated plasmids from EcN clones positive for EPA/DHA genes and a negative control (plasmid without insert) were digested with NotI-HF. Digested and undigested plasmids were separated in 1% agarose gel.

Fatty acid analysis: The bacterial cells from 200 mL of EcN transformants, EcN (negative control), and S. baltica MAC1 (positive control) were harvested by centrifugation at 8000 r.p.m for 17 min at 4° C., then freeze-dried overnight. Total lipids from the freeze-dried cells were extracted as described previously (Amiri-Jami & Griffiths, 2010). The fatty acid methyl esters (FAMs) were analyzed using an automated Agilent 6890 Gas Chromatography (GC) system (Agilent, Palo Alto, USA) and a Varian 3800 GC/Saturn2000 ion trap mass spectrometer in external El mode (GC-MS) (Varian, Mississauga, Canada) as described previously (Amiri-Jami & Griffiths, 2010). Compounds were detected by comparison with relative retention time and mass spectra of pure EPA and DHA standards (Sigma Aldrich, Oakville, Canada).

RNA extraction and cDNA transcription: RNA was isolated from transformant EcN 1917 (positive for EPA/DHA production), EcN (negative control) and S. baltica MAC1 (positive control) grown in LBCm, LB and MB, respectively and incubated at 15° C. to an absorbance of 0.35 (A600). The total RNA was extracted using an RNA purification kit (Norgen bioTek, Thorold, ON, Canada) according to the manufacturer's instructions. DNA was eliminated from extracted total RNA using DNase I recombinant RNase-free (Roche Applied Science, Laval, QC, Canada) as follows: 20 μL of total RNA was incubated at 37° C. for 20 min with 10 U of DNase I, 10 U of RNase inhibitor, and 5 μL of incubation buffer in a total volume of 50 μL. The RNA was then purified using the cleanup kit (Qiagen, Ontario, Canada). Purified RNA was transcribed into cDNA using the TruScript™ First Strand cDNA Synthesis Kit (Norgen bioTek, Thorold, ON, Canada) according to the manufacturer's instructions. For control samples, reverse transcriptase was replaced by RNase-free water. The cDNA synthesis was performed in a Gradient Master-cycler (Eppendorf, Mississauga, ON, Canada) under the following conditions: 25° C. for 5 min, 50° C. for 60 min and 70° C. for 15 min. The cDNA was stored at −20° C. until further use.

Quantitative real-time PCR: The expression of EPA/DHA genes (pfaA, pfaB, pfaC, pfaD, pfaE) was determined by RT-qPCR. The primers used for RT-qPCR are described in Table 2. The housekeeping gene 16S rRNA was used for normalization of the expression as described by Pfaffl, 2001.

Real-time PCR was carried out in a ViiA™7 detection system (Applied Biosystems). The reaction mixture contained 1.5 μL cDNA, 1 μL of each primers (final concentration of 600 nM), 10 μL SYBR Select PCR Master Mix (Applied Biosystems, Carlsbad, Calif.), and 6.5 μL of DNase/RNase-free deionized water. The amplification conditions were as follows: 95° C. for 10 min, 40 cycles of 95° C. for 15 sec, annealing/extension at optimal temperature for each pair of primers (55° C. for pfa B-E genes, 58.3° C. for pfaA gene and 60° C. for 16s rRNA gene) for 1 min. The PCR conditions used for the pfaA gene were different from other genes; being 95° C. for 10 min followed by 40 cycles of 94° C. for 30 sec, 58.3° C. for 1 min and 72° C. for 1 min. Thermal cycling, fluorescent data collection, and data analyses were carried out by ViiA™ 7 Software according to the manufacturer's instructions.

Real-time PCR amplification efficiencies (Table 3) were calculated from the slope of the standard curve using the data collected from serial dilutions of the template DNA for each gene according to the equation described by Rasmussen (2001) and Pfaffl (2001):

E=10^{[-1/slope]}

The relative changes in gene expression were calculated as described previously (Pfaffl, 2001) using the formula:

Ratio=(E target)^{delta Ct target (control-sample)}/(E ref)^{delta Ct ref (control-sample)}

The cycle threshold (Ct) value is the PCR cycle at which an increase of fluorescence is first detected above the baseline signal. The experiment was performed in duplicate. A relative increase or decrease in transcription value of more than two-fold was considered as significant up- or down-regulation, respectively (Pfaffl, 2001).

### Results and Discussion

Recombinant production of EPA/DHA by E. coli Nissle 1917: E. coli Nissle 1917 was transformed with pfBS-PS carrying the 20 kbp EPA/DHA gene cluster. Chloramphenicol-resistant transformants were tested for pfaA and pfaD genes by PCR. Digestion of isolated plasmid DNA from the positive clone (gEcN) for pfaA and pfaD genes with Notl resulted in two bands of size 20 kbp and 8 kbp corresponding to the EPA/DHA gene cluster and plasmid pCC1FOS, respectively. Fatty acid methyl ester analysis of gEcN by GC showed two extra peaks at retention times corresponding to EPA and DHA standards. No extra peaks were detected in the fatty acid profile of the negative control (EcN). The EPA/DHA peaks detected by GC were further analyzed by mass spectrometry. The GC-MS results confirmed that the large extra peak (EPA) detected by GC corresponded to the molecular mass of EPA. However, the small peak identified for DHA by GC was not detected by GC-MS due to its very low concentration.

The transgenic E. coli Nissle produced EPA when grown at 10° C. (16.52±1.4 mg g-1 cell dry weight), 15° C. (31.36±0.25 mg g-1 cell dry weight), 20° C. (13.71±2.8 mg g-1 cell dry weight), 25° C. (11.33±0.44 mg g-1 cell dry weight) or 30° C. (0.668±0.073 mg g-1 cell dry weight). Although DHA was also produced at all these temperatures, it comprised less than 0.2% of total extracted fatty acids. Transcriptomic analysis using Reverse Transcription qPCR showed up-regulation of the entire gene cluster in E. coli Nissle. Among EPA/DHA genes, pfaB, pfaC and pfaD were over-expressed (expression ratio of 181.9, 39.86 and 131.61, respectively) as compared to pfaA (expression ratio of 3.40) and pfaE (expression ratio of 4.05). Accordingly, the EPA/DHA-producing probiotic E. coli Nissle may be used as a safe and economical source for the production of EPA and DHA.

Recombinant E. coli Nissle 1917 produced EPA at all tested temperatures and traces of DHA were observed following growth at 15° C. or 20° C. Transformant gEcN produced 31.36±0.25 mg EPA g-1 of cell dry weight (CDW) at 15° C., which is almost 16 times greater than EPA produced by the marine bacterium S. baltica MAC1 (1.96±0.05 mg EPA g-1 CDW) at the same temperature (FIG. 1). The amount of EPA produced by gEcN ranged from 0.668±0.073 to 31.36±0.25 mg EPA g-1 CDW when it was grown at different temperatures (FIG. 2). The level of EPA produced by gEcN was significantly higher at all temperatures tested compared to the strain from which the genes were originally cloned, S. baltica MAC1.

Transformation of the EPA/DHA gene cluster isolated from different marine bacteria to E. coli strains has been reported (Amiri-Jami et al., 2014; Amiri-Jami & Griffiths 2010; Orikasa et al., 2009; Okuyama et al., 2007). However, there is need for a safe, alternative and economical source for the industrial and pharmaceutical production of EPA and DHA. The EPA/DHA gene cluster has been transferred to Lactococcus lactis subsp. cremoris, a food grade bacterium, however production of EPA and DHA was very low (Amiri-Jami & Griffiths, 2014).

In contrast, when the large 20 kbp EPA/DHA gene cluster was transferred to E. coli Nissle 1917 (EcN), genetically modified EcN was able to produce high levels of EPA and traces of DHA following growth at different temperatures. EcN has been applied as a safe microorganism and a probiotic reagent against intestinal disorders for many decades. The safe human use of modified EcN to deliver nutrients such as beta-carotene (Miller et al., 2013), and proteins like defensin (Seo et al., 2012) and ferritin (Hill et al., 2011) has been reported.

Comparison of EPA/DHA genes Expression in gEcN and S. baltica MAC1: The expression of EPA/DHA genes was studied in gEcN and S. baltica MAC1 grown at 15° C. Wild-type E. coli Nissle 1917 was used as a negative control. The transcription of the EPA/DHA genes (pfaA, pfaB, pfaC, pfaD, pfaE) was investigated and shown to be significantly up-regulated in gEcN compared to S. baltica MAC1 (Table 3). Among all five genes, pfaA had the lowest (ratio of 3.40; Table 3) and pfaB had the highest (ratio of 181.9; Table 3) expression ratio. These results indicate that the meaningful increase in EPA levels by gEcN is due to changes in expression of all corresponding genes when compared to S. baltica MAC1. In addition, genes pfaB, pfaD and pfaC were highly over expressed.

The expression of EPA/DHA genes in gEcN was also compared to that of S. baltica MAC1. In S. baltica, MAC1 genes are located in the genomic DNA, while in gEcN, the genes are plasmid-mediated. These data show that there was a significant increase in expression of all five genes, responsible for the production of EPA/DHA in gEcN compared to S. baltica MAC1. This could be related to the higher copy number of the EPA/DHA genes when they are carried in a plasmid compared to when they are chromosomally mediated (one copy for each gene). Among the five genes, pfaB was highly up-regulated compared to other genes. In addition, it has been reported that pfaB gene encodes beta-hydroxyacyl-ACP dehydratase (HD), beta-ketoacyl-ACP synthase (KS) and acyltransferase (AT) (Amiri-Jami & Griffiths 2010). Allen & Bartlett (2002) have described the role of these domains in the synthesis of EPA in detail. The activities of the HD domains introduce multiple double bonds into a single acyl chain in the absence of desaturation reactions. Moreover, intermediates in the biosynthetic process are presumably bound to acyl carrier protein (ACP) domains as thioesters with AT domains being required for the loading of the starter and extender units while KS domains are involved in condensation reactions. Orikasa et al. (2009) reported that pfaB gene encodes the key enzyme determining the final product in EPA/DHA biosynthesis. This study revealed that up-regulation of pfaB domains might be responsible for higher production of EPA. However, the level of DHA production by gEcN remained low similar to that of S. baltica MAC1. Accordingly, the biosynthesis of DHA could be influenced by pfa gene products other than pfaB. In addition, it is possible that the host factor(s) might have some regulatory functions on the biosynthesis of EPA and DHA.

The present example describes the genetic engineering of a commercial probiotic bacterium with the ability to produce high levels of EPA at different temperatures in a simple medium. Since EcN is a normal and ecologically important inhabitant of the human and animal intestinal tracts, EPA/DHA-producing E. coli Nissle or fat isolated from gEcN may be a cost effective, sustainable and convenient source for industrial production of omega 3 fatty acids for human consumption and pharmaceutical applications. Moreover, recombinant EPA producing E. coli Nissle is also expected to be useful for studying the delivery and production of EPA in the human intestine and its immunostimulatory effects.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

All publications, accession numbers describing biological sequences, patents and patent applications are herein incorporated by reference in their entirety to the same extent as if each individual publication, accession number describing a biological sequence, patent or patent application was specifically and individually indicated to be incorporated by reference in its entirety.

### REFERENCES

1. Abbadi A, Domergue F, Bauer J, Napier J A, Welti R, Zahringer U, Cirpus P & Heinz E (2004) Biosynthesis of very-long-chain polyunsaturated fatty acids in transgenic oilseeds: constraints on their accumulation. Plant Cell 16:2734-2748.
2. Adarme-Vega T, Thomas-Hall S & Schenk P M (2014) Towards sustainable sources for omega-3 fatty acids production. Curr Opin Biotechnol 26: 14-18.
3. Allen E E & Bartlett D H (2002) Structure and regulation of the omega-3 polyunsaturated fatty acid synthase genes from the deep-sea bacterium. Photobacterium profundum. strain SS9 Microbiology 148: 1903-1913.
4. Altenhoefer A, Oswald S, Sonnenborn U, Enders C, Schulze J, Hacker J & Oelschlaeger T A (2004) The probiotic Escherichia coli strain Nissle 1917 interferes with invasion of human intestinal epithelial cells by different enteroinvasive bacterial pathogens. FEMS Immunol Med Microbiol 40: 223-229.
5. Amiri-Jami M & Griffiths M W (2010) Recombinant production of omega-3 fatty acids in Escherichia coli using a gene cluster isolated from Shewanella baltica MAC1. J Appl Microbiol 109: 1897-1905.
6. Amiri-Jami M, Lapointe G & Griffiths M W (2014) Engineering of EPA/DHA omega-3 fatty acid production by lactococcus lactis subsp. cremoris. MG1363 Appl Microbiol Biotechnol 98: 3071-80.
7. Bayoumi M A & Griffiths M W (2012) In vitro inhibition of expression of virulence genes responsible for colonization and systemic spread of enteric pathogens using Bifidobacterium bifidum secreted molecules. Int J Food Microbiol 156: 255-263.
8. Bowman J P, Gosink J J, McCammon S A, Lewis T E, Nichols D S, Nichols P D, Skerratt J H, Staley J T & McMeekin T A (1998) Colwellia demingiae sp. nov., Colwellia hornerae sp. nov., Colwellia rossensis and Colwellia psychrotopica sp. nov. psychrophilic Antarctic species with the ability to synthesize docosahexaenoic acid. Int J Syst Bacteriol 48:1171-1180.
9. Helwig U, Lammers K M, Rizzello F et al. (2006) Lactobacilli, Bifidobacteria and E. coli Nissle induce pro- and anti-inflammatory cytokines in peripheral blood mononuclear cells. World J Gastroenterol 12: 5978.
10. Hernando-Harder A C, von Buenau R, Nadarajah M, Singer M V & Harder H (2008) Influence of E. coli strain Nissle 1917 (EcN) on intestinal gas dynamics and abdominal sensation. Dig Dis Sci 53: 443-450.
11. Hill P J, Stritzker J, Scadeng M, Geissinger U, Haddad D, Basse-Lusebrink T, Gbureck U, Jakob P & Szalay A A (2011) Magnetic Resonance Imaging of Tumors Colonized with Bacterial Ferritin-Expressing Escherichia coli. PLoS One 6: e25409.
12. Huijsdens X, Linskens R, Mak M, Meuwissen S, Vandenbroucke-Grauls C & Savelkoul P (2002) Quantification of bacteria adherent to gastrointestinal mucosa by real-time PCR. J Clin Microbiol 40: 4423-4427.
13. Kris-Etherton P, Harris W S & Appel L J (2002) Fish consumption, fish oil, omega-3 fatty acids, and cardiovascular disease. Circulation 106: 2747-2757.
14. Lopez N R, Haslam R P, Usher S L, Napier J A & Sayanova O (2013)

Reconstitution of EPA and DHA biosynthesis in Arabidopsis: iterative metabolic engineering for the synthesis of n-3 LC-PUFAs in transgenic plants. Metab Eng 17:30-41.
15. Medellin-Pena M, Wang H, Johnson R, Anand S & Griffiths M W (2007) Probiotics Affect Virulence-Related Gene Expression in Escherichia coli O157: H7. Appl Environ Microbiol 73:4259-4267.Miller J, Harrison Miller J, Harrison M, D'Andrea A, Endsley A, Yin F, Kodukula K & Watson D (2013) β-Carotene Biosynthesis in Probiotic Bacteria. Probiotics & Antimicro Prot 5: 69-80.
16. Nissle A (1918) Die antagonistische Behandlung chronischer Darmstorungen mit Colibakterien. Med Klin 2: 29-30.
17. Oelschlaeger T A (2010) Mechanisms of probiotic actions—A review. Int J Med Microbiol 300: 57-62.
18. Ohland C L & Macnaughton W K (2010) Probiotic bacteria and intestinal epithelial barrier function. Am J Physiol Gastrointest liver Physiol 298: G807.
19. Okuyama H, Orikasa Y, Nishida T, Watanabe K, Morita N, Okuyama H, Orikasa Y, Nishida T, Watanabe K & Morita N (2007) Bacterial genes responsible for the biosynthesis of eicosapentaenoic and docosahexaenoic Acids and their Heterologous expression. Appl Environ Microbiol 73: 665.
20. Orikasa Y, Yamada A, Yu R, Ito Y, Nishida T, Yumoto I, Watanabe K & Okuyama H (2004) Characterization of the eicosapentaenoic acid biosynthesis gene cluster from Shewanella sp. strain SCRC-2738. Cell Mol Bio 50:625-630.
21. Orikasa Y, Tanaka M, Sugihara S et al. (2009) pfaB products determine the molecular species produced in bacterial polyunsaturated fatty acid biosynthesis. FEMS Microbiol Lett 295: 170-176.
22. Pfaffl M W (2001) A new mathematical model for relative quantification in real-time RT-PCR. Nucleic Acids Res 29: e45.
23. Ramakrishnan U, Stein AD, Parra-Cabrera S, Wang M, Imhoff-Kunsch B, Juarez-Marquez S, Rivera J & Martorell R (2010) Effects of docosahexaenoic acid supplementation during pregnancy on gestational age and size at birth: Randomized, double-blind, placebo-controlled trial in Mexico. Food Nutr Bull 31: S108-S116.
24. Rasmussen R (2001) Quantification on the LightCycler. Rapid Cycle Real-time PCR, Methods and Applications (Meuer S, Wittwer C & Nakagawara K, eds), pp. 21-34. Springer Press, Heidelberg.
25. Seo E J, Weibel S, Wehkamp J & Oelschlaeger T A (2012) Construction of recombinant E. coli Nissle 1917 (EcN) strains for the expression and secretion of defensins. Int J Med Microbiol 302: 276-287.
26. Swanson D, Block R & Mousa S A (2012) Omega-3 fatty acids EPA and DHA: health benefits throughout life. Adv Nutr (Bethesda, Md.) 3: 1-7.
27. Takeyama H, Takeda D, Yazawa K, Yamada A & Matsunaga T (1997) Expression of the eicosapentaenoic acid synthesis gene cluster from Shewanella sp. in a transgenic marine cyanobacterium, Synechococcus sp. Microbiology 143: 2725-2731.
28. Troge A, Scheppach W, Schroeder B O, Rund S A, Heuner K, Wehkamp J, Stange E F & Oelschlaeger T A (2012) More than a marine propeller—the flagellum of the probiotic Escherichia coli strain Nissle 1917 is the major adhesin mediating binding to human mucus. Int J Med Microbiol 302: 304-314.
29. Tully A M, Roche H M, Doyle R, Fallon C, Bruce I, Lawlor B, Coakley D & Gibney M J (2003) Low serum cholesteryl ester-docosahexaenoic acid levels in Alzheimer's disease: a case-control study. Br J Nutr 89: 483.
30. Westendorf A M, Gunzer F, Deppenmeier S, Tapadar D, Hunger J K, Schmidt M A, Buer J & Bruder D (2005) Intestinal immunity of Escherichia coli Nissle 1917: a safe carrier for therapeutic molecules. FEMS Immunol Med Microbiol 43: 373-384.
31. Yagoob P & Calder P C (2007) Fatty acids and immune function: new insights into mechanisms. Br J Nutr 98: 41.
32. Yu R, Yamada A, Watanabe K, Yazawa K, Takeyama H, Matsunaga T & Kurane R (2000) Production of eicosapentaenoic acid by a recombinant marine cyanobacterium, Synechococcus sp. Lipids 35:1061-1064.

TABLE 1
Bacterial strains and plasmids used in this study

| Source | Description | Strain or plasmid |
|---|---|---|
| CRIFSCC* | EPA+, DHA+, marine bacterium | S. baltica MAC1 |
| CRIFSCC | EPA− , DHA− bacteria | E. coli Nissle 1917 |
| CRIFSCC | pfBS-PS (pCC1FOS + EpA/DHA | E. coli EPI300T1 |
| | gene cluster) | |
| This study | pfBS-PS | gEcN |
| | pCC1FOS + 20 kbp EPA/DHA | PfBS-PS plasmid |
| CRIFSCC | cluster | |

*Canadian Research Institute for Food Safety Culture Collection

TABLE 2
Target genes and primers used for RT-qPCR

| | Primer pairs (forward | Product | Annealing | Reference |
|---|---|---|---|---|
| Gene | and reverse) and (SEQ ID NO) | (bp) | temp(° C.) | |
| A | F: GGTGTSGGYGGTGGTCAR (7) | 1800 | 58.3 | (Amiri-Jami |
| | R: CTCACCRAARCTRTGRCC (8) | | | & Griffiths, |
| | | | | 2010) |
| B | F: TCACCAGTTAGAACGTGAAG (9) | 110 | 55 | This study |
| | R: TAATCGCTTGCTGACTTAGG (10) | | | |
| C | F: CGACTGACTATTTGCTGGTA (11) | 101 | 55 | This study |
| | R: GTCCACAGGAATGTCGTATT (12) | | | |
| D | F: GAAATGGCCGATGTGACTAT (13) | 131 | 55 | This study |
| | R: TCGTAACGGGTGTAGATTTC (14) | | | |
| E | F: CCAATCAATAACCAATCGCC (15) | 146 | 55 | This study |
| | R: GTGTTATCTCGCTATGCTGA (16) | | | |
| 16S rRNA | F: CATGCCGCGTGTATGAAGAA (17) | 96 | 60 | (Huijsdens |
| housekeeping | R: CGGGTAACGTCAATGAGCAAA | | | et al., |
| | (18) | | | 2002) |
| Universal 16S | F: ACTCCTACGGGAGGCAGCAGT | 199 | 60 | |
| rRNA (MAC1) | (19) | | | |
| | R: GTATTACCGCGGCTGCTGGCAC | | | |
| | (20) | | | |

TABLE 3
Expression ratio of pfaA-E genes of gEcN normalized to the expression
of 16S- rRNA gene and compared to S. baltica MAC1

| | PCR | Cₜ | Cₜ | Expression |
|---|---|---|---|---|
| Gene | Efficiency | value/gEcN | value/S.MAC1 | Ratio |
| pfaA | 2.02 | 32.71 ± 0.35 | 35.53 ± 0.89 | 3.40 |
| pfaB | 1.8 | 24.05 ± 0.005 | 34.19 ± 1.13 | 181.90 |
| pfaC | 1.74 | 23.14 ± 0.19 | 31.16 ± 0.32 | 39.86 |
| pfaD | 1.81 | 24.40 ± 0.54 | 33.90 ± 1.15 | 131.61 |
| pfaE | 1.83 | 34.18 ± 0.06 | 37.75 ± 0.005 | 4.05 |
| 16s rRNA | 1.92 | 4.10 ± 0.06 | 5.26 ± 0.03 | NA* |

*Not Applicable
Cₜ: cycle threshold

SEQUENCE LISTING

21Kb EPA/DHA gene cluster SEQ ID NO: 1
CAGAGCACCACTATGCCTTCAAGCCTTCGCCTTGTCTAA
AGCGCGTTGAACTCCCGCTGAATGAACAGATATTTAAT
ACGATTGGTATTAAACTGGCTGATAACAAGATAACGCGC
AGAAAGAATTATGGCTAAACGCAATCGCAGATCTTAGC
GGTTTAGGTGAGTTTACTGGCGCGAAGAAAGCACTCTCT
TTCGCCGTTAATAATCAGCAAGTAAGCTCTCTATGCTG
CTCAACTGCGCGGTTAGCGTTATGGGAAGCGCGGCATTA
CCGGCATGCGCTAAGCTAATGGCAAAGCGATACTCCTC
GGTCAAACGACCAAAATGCGTTTGCCAATGCAAGGAGTC
AGCTAAATCCAGTGTGTGTATTGGGATTTGTAGCGGGA
CTTGTAATGGAATATGTGGATAAACATTAAGCATTGAAG
CTACGCCGCAGGCTTGCCTCAATCCGGCATCTTGGCAT
GATAACTCAAGCGATTCTGCCGCCAATTGCTCAAACGGC
TTAAGCTCAAAGGCAAAGGATTTGAGCGACTGCGCTAA
ACCTAAACCAGTCGCCTTAATATAGGACTCCTTTAGCGC
CCATAAATCGAAAAAGCGCTGTCGCTGACTCGCTTCAT
CGGCGAGAGCGAGCAAGGCTGCAGTTTCTTGGGGAGAAA
AATAGTGATTTAGGATTGGATAGATATCCGTCTTCGGC
CGCGACCTTTCAATATCCACCCCAAATAAACCAGACTCA
ACGCCATCAAACTGAGCCACACCAATCAATAACCAATC
GCCGCTATGGCTTAGGTTAAACTCGAGACCAGTTTGCTG
CCCTAATGTCGCCGTTAAACTCGGTTTGCCCTTAGCGC
CATATTCAAAACACCACTCATGTGGCGATAAATCAGCAT
AGCGAGATAACACGGTCCGTAGCGCCGCCCTCACCTGT
AACCCCTTTATTTGCACCTTGGGATCGCGATAACGCCTG
ACCTTTGCGAGTTCATCATCACTTAACCAAGACTCGGC
AAGTGATGTTTGCGCCGCTGAAATATCGTTCAGTGGAAT
AAAAAATAGATCCACATTCACGCCGTTTTTGCTAGATG
TTAGAGAGTCGGTCATTCGAGAGCCTAGCTGTACAAAGC
CCTGCGGCTGATGAGTTATCATTGGCTGGCTTTTTGGA
GAGCAGTTAGCAACATTCTTATCTGGTTCATTATTGTCT
AGGTTCATCATTATCTGAGTTCATTATGCATCTTAAAT
AGTTCCAACTAAGCCCTATTTCAATTCTTAGCCGCACTC
TAATAAGAGCAATTCTTAGAACGACTATTAACTCTATC
CTAAGCGCAAAATTTTGCTGCAATTGCGCCAAGGTTCCC
CCCCAAAAAAAATTAGCAGGATTAGCCACACAGCCTCA
TTCATGACTAAAGGGACGCAAGCCAATATTGGGCTATTT
TACCCCGATAACTCAAAAAGCTGCCTTCATCGCCGCTA
CAAATATAAATCTTCCACTATCAAAGTATTAAACAGCCC
AAGCGGCCAGCGAAAACCCAACATAACGCACAGAAAAT
ACCATTACTCAACGAAAGCCAATAAATTCCAGTCCCAGA
TGATTGTATCGACCCGAAACCTCAGGTAATCTGCATCC
CCATTAAGTGACACAGATGATAAGCACAGGTTTTAAGAT
GAGTGTCACGACATTAGTCATAAATGTCAGTCGTGACT
CAGCCAAAATCGCTTATCAGTCCCAATAGATTATGTCGG
ATGATAATTATTTTTAATGGCAACTCGCGACTCTCAAT
CTAAAGCGCCGTCATACGCCCCCAAGAGCACGACTCCAG
TGGCGCAAAAAGACAGCACACCTCGGCATCGCAACGCC
ACCACTACGCCTGAAATGCGACTCTTTATCCAACAATCA
GATCTGAGCGTGAGCCAGCTGGCAAAGATTTTAAATAT
TACCGAGGCAACGGTGCGTAAGTGGCGTAAGCGTGAGTC
CATCAGCAACAGTTCGAACACGCCCCACCATCTCAACA
CGACGCTCACGCCGATGGAAGAATACGTGGTGGTGGGAT
TACGTTACCAACTCAGACTCACCTTAGATAGATTGCTC
CACGTCACCCAAACCTACATTAATCCCAATGTGTCGCGC
TCCGGCCTTGCCCGTTGCTTAAAGCGCTACGGCATATC
GCGGCTCGATGAGTTTGAAGCACCACAAGTACCCGAACG
CTATTTTAATCAATTACCTGTGACTCAAGGCAGCGATA
TCCAAACCTATACGGTCAACCCCGAAACCTTGGCCAAAG
CCCTCGCATTGCCGAGTACCGACGGCAATACTGTGGTG
CAAGTGGTATCACTCACCATACCGCCGCAGCTTACAGAG
CAAGCTCCAAGCTCAGTGCTATTAGGCATAGATACGGC
AAGTGATTGGATTTACCTCGATATTTATCAAGACAGCAA
TACTCAAGCGACCAATAGATATATCGCCTATGTCCTCC
GACATGGGCCGTTTCATTTACGAAAGTTGCTCGTTCGCA
ACTATCACACCTTCTTAGCCCGTTTTCCCGGTGCCCAT
GGCACGCCAAAGACAAACGCGGCTGAACCCCAAAACAAG
GTCACCGTATCCAGGTCGACCCGTGGAGACTCTTTATG
AGCCATACCCCTTCTGTACCTAATTCTGCGACTGAGTCA
AAAAAAGATAAACGACTCAACAAACGTTTGAAAGATAT
GCCCGTTGCCATCGTCGGCATGGCCAGTATCTTCGCCAA
CTCGCGTTATTTAAATAAATTTTGGGATTTGATCAGCG
AAAAAATCGATGCCATTACTGACATCCCAGACACCCATT
GGCGCGCCGAAGATTACTACGATGCAGACAAAAGCAAA
GCCGATAAAAGCTACTGTAAACGCGGTGGTTTTTTGCCT
GAAGTCGACTTCAACCCAATGGAATTTGGTCTGCCGCC
CAATATTCTTGAGCTAACAGATACCTCGCAACTGCTGTC
GCTGATTGTCGCCAAAGAAGTGCTCGCCGATGCCAATC
TGCCCACCGATTATGATCGCGATCGTATCGGCATCACGC
TAGGCGTGGGCGGTGGTCAAAAAATCAGTCAGAGCTTG
AACTCGCGCCTGCAATATCCAGTATTGAAAAAAGTATTC
AAAAGCAGCGGCCTGAGCGATGAAGACAGCGAAATGCT
GATCAAAAAGTTCCAAGACCAATATATCCACTGGGAAGA
AAACTCTTTCCCAGGGTCCTTAGGCAACGTGATTGCAG
GCCGTATCGCCAACCGTTTTGATTTTGGCGGCATGAACT
GTGTGGTCGATGCTGCCTGCGCGGGCTCACTTGCCGCT
ATGCGTATGGCGTTGACTGAACTGACCGAAGGTCGCAGC
GACATGATGATCACCGGCGGCGTCTGTACCGACAACTC
GCCGTCCATGTATATGAGCTTCTCAAAAACGCCTGCCTT
CACTACCAATGAAACCATTCAACCCTTTGATATCGATT
CAAAGGGCATGATGATCGGCGAAGGTATTGGCATGGTAG
CACTTAAGCGCCTTGAAGATGCCGAGCGCGATGGCGAC
CGGATTTATGCCGTCATCAAAGGCGTTGGCGCCTCATCG
GACGGTAAATTTAAGAGTATTTATGCGCCGCGCCCTGA
AGGCCAAGCTAAAGCATTGGAGCGCGCCTACGACGACGC
GGGTTTTGCCCCGCACAGCATTGGCTTAGTTGAAGCCC
ATGGCACGGGCACTGCCGCAGGTGATGTGGCCGAATTCA
ATGGCTTAAAATCGGTATTTGCCCAAGGCAACGACACC
AATCAACATATCGCGTTAGGTTCAGTGAAATCCCAAGTG
GGCCACACTAAATCAACCGCAGGTACTGCTGGGGTAAT
CAAAGCCGCGCTGGCGCTGCACCACAAGGTATTACCTGC
GACCATTAACGTCAGCAAGCCTAATCCAAAACTGAATA
TCGAAAGCTCACCATTCTATTTAAATACCGAAACGCGCC
CTTGGCTGCAACGCACTGACGGTACGCCGCGCCGTGCT
GGCATAAGTTCCTTTGGTTTTGGCGGCACTAACTTCCAT
CTCGTATTAGAAGAATACAAACCCGAGCACAGCCGTGA
CGAGCAATATCGTCAGCGCAGTGTGCCGCAAACGCTATT
ATTTGCCGCCGCCAATAAAGCCGCGCTGCTTAGCGAGT
TAAAAGCTGCGCTGAGCCAAAGCTTGAATACGAACGCGA
ATAAGAGCAGCGCTGCGAGCCTGAACGCTATCGCTCAG
CAATATCCGCTACGCGCGCTGGCAGACACAGATGCCCGT
TTAGGTTTTGTGGCTAAGGATATCGCTCAGCTGCAAGC
TCAGTTGAATCAAGCCATCTCTCATCTAGAAAGCAGTGC
GCATTTAGAAAGCGGGCTCCTAGATACCAGTGCCAGCG
AGGCGTGGCAGTTACCTTCTGGGATCAACTACCGCGCAA
AAGCTTTAGTTGCTAAAGATGAATCAAAGAAAGTGGCC
GCTCTGTTTGCCGGTCAAGGTTCACAGTACCTGAACATG
GGCCGCGAACTCGCCTGCCATTTCCCTGAAATGCGCCA
ACAAGTGATGGCCAGCGATAAGGTATTTGCTCACCACGG
CCAAACGCCGTTATCCAACATCCTTTATCCTATTCCGG
CATTCGATGCCGATGCGGTTAAAGCCCAAGAAGCGGCGC
TGACCAATACCCTGTTCGCCCAAAGCGCCATAGGCGCG
GTTTCAATGGCGCAATATTCACTATTAACTCAGGCAGGT
TTTGCCCCAGATATGGTGGCGGGTCACAGCTTTGGTGA
ATTGTCAGCCCTTTGCGCGGCTGGCGTGATTTCGAGTGT
TGATTACGTCGAACTGGCCTTCGCCCGTGGGCACGCCA
TGGCGCAAGTGCCAAGTGATACTGACGCTCAAGTCGATT
TAGGCAAGATGTTTGCCATCATCCTCAAACAAAAGAGT
GATATCGATGCGCTTAATCGCTGTTTAGCTCAGTTTGAT
GGCGTTAAAATTGCCAACTACAACGCGCCGACACAATT
AGTCATCGCAGGTGGCACAGAGCAAACTCAGCTGGCTGC
CAAAGCTATTAGCGAACAGGGCTTTAAAGCGATTGCCC
TGCCCGTCTCTGGCGCGTTCCACACCCCCTTGGTTGGGC
ACGCACAAAAGCCATTTGCCAAGGCCATTGATAAAGCT
AAATTCAGCGCGCCTTCAATCGCACTTTATGCCAACGGC
ACAGGTCAATTGCACCCAAGCGATGGCAAAGCGATTAA
AGCAGAATTCAAACAACACATGCTGCAATCGGTTCGTTT
TAGCGAACAACTGCAGGCCATGTATGATGCTGGCGCCC
GTGTGTTTGTTGAGTTTGGCCCTAAGAACATACTACAAA
AGCTCGTTGAAAATACCTTAAGTGAACACTTAAATGAG
CTTTGCCTTGTCAGCATGAACCCGAACCCTAAGGGCGAC
AGTGACAGTCAATTACGCTTAGCCGCTGTGCAACTCGC
GGTAGCCGGTGTGGCGTTAACTGAGGTTGACCCCTATCA
AGCCGTCACGTCACAGGAGATTGCCGAGCGTGAAGCGC
CATCGGCGATGAACATCAAACTCACCGCCACTAACCACA
TCAGTGCCGCAACCCAAGCTAAGATGGCGAAATCTCTC
GCCACAGGCAGCGTGACTAGCCAAGTGCAATATGTGGAT
CGCATCGTTGAAACGATTCTCGAAAAAGAAGTCGAAAA
AATCGTTCAGAAAGAAGTCATAGTCGAAAAAGTGGTTGA
GAAAATAATAGAAGTGGAAGCGAATCAAGTGGCAGCTG
TTGAAATGAAACAAAAACTCCTAAGCGTGACGCAAGGCT
TAAATCACCAACAAGCGACGGCGCAGATGAGCCCAAGC
ACAGCAAACGTCAGCGGCGATGCGTTAACGGCATTTTTC
AGCGCACAATCTCAAGCGGCGCAGTTACATCAACAGTT
TTTAGCCATCCCGCAGCAATATGGCGATACTTTCACGAC
ACTGATGACAGAACAAGCCAAGATGGCGAGCCTTGGCA
TTGCAATCCCAGAAAGCCTGCAACGCTCGATGGAAATGT
TCCACCAGCATCAAGCGCAAACTCTGCAAAGCCACGCC
GAATTTATGCAACTGCAAAGCAGTAGCAGCCAAGCGGCC
TTGGCCATGTTAAACAATGCGCCGATTAACTTTACCCC
AGCGGTTGCAAGTCAGCCACAAGCAACTGTACCTGTTGC
TCCTGCACCTGTTGCTGCATCGACAGTGGCACACAATG
CCGCACCAGTCGCGGCTCAGGCTGTTGCGACAAGACCAG
CGGTTAGCACACCCGTTCCTCCTGTTGTGCAAACCGCT
CCCGTTGCTTATGCGCCTGCTGCGACAGTACAAGTTGCG
CCAGCAGCTCCTGCCGCACCCGCATTAGTCATGCCTGC
TGTGGTGATGCCAGAAGTGACACCTGTGGCACCAACAAT
GAACGGTTTAAGTGCTGAAAAAGTACAGCAAACAATGA
TGGCTGTGGTGGCTGGCAAGACAGGCTATCCGACTGAAA
TGCTGGAGCTCAGCATGGATATGGAAGCCGATCTCGGT
ATCGATTCAATTAAGCGCGTTGAAATCTTAGGCACAGTG
CAAGACGAGCTGCCGAGTTTACCTGAACTCAGCCCAGA
AGATTTGGCTGAGTGCCGTACCTTGGGTGAAATCGTAAG
CTATATGAATAGCAAATTACCTGCAGCGGGAACTATTG
CTTCGACAGCAGCGCCAGTTGCTCAAACCATTGCCGCTC
CTGCATCTAACGCTTTAAGCGCTGCATTAGTTCAACAA
ACTATGATGGCTGTGGTGGCCGACAAGACAGGCTACCCC
ACTGAAATGCTGGAACTTGGCATGGATATGGAAGCCGA
TTTAGGTATCGATTCTATTAAGCGCGTTGAGATTTTAGG
CACAGTGCAAGACGAACTGCCGGGTTTACCCGAACTTA
GCCCAGAAGATTTGGCCGAATGTCGCACCTTAGGTGAAA
TCGTTACCTATATGAACTCTAAACTGCCAGCAGCGGGT
TCAGTTGTAGTTTCTACAACAGCACAAGCCGCGCCTGCC
GACAGCGGTTTAAGCGCTGAGTTAGTGCATTCTTCTGA
AATCGAGAGCACCATGATGGCTGTGGTGGCCGACAAGAC
AGGCTATCCGACTGAAATGCTAGAACTGAGCATGGATA
TGGAAGCCGATCTCGGTATCGACTCCATCAAGCGCGTTG
AAATCCTAGGCACAGTGCAAGACGAACTGCCGGGCTTA
CCTGAACTTAGCCCAGAAGATTTGGCCGAGTGTCGTACC
TTGGGTGAAATCGTTAGCTATATGAATAGCAAATTACC
AGCAGCAGGAGTTATTGCTTCGACAGCTGCGCCAGTTGC
TCAAACCATTGCAGCTCCTGCCGCAATCGGTTTAAGCG
CTGCATTAGTGCAACAAACTATGATGGCTGTGGTGGCTG
ACAAGACAGGTTACCCCACTGAAATGCTGGAACTCAGC
ATGGATATGGAAGCCGATTTAGGTATCGATTCTATCAAG
CGCGTTGAAATTTTAGGCACAGTGCAAGACGAACTTCC
TGGCTTACCTGAACTCAATCCTGAAGATCTAGCCGAGTG
TCGCACCTTAGGTGAAATCGTTGACTACATGAACAGCA
AACTACCTGCGGTTGGCTCGACTTCAACTATAACAGCAG
CACAGGTCACTGTTACCGCTACAATAAATAACGGCTTA
AGTGCTGAAAAAATCCAACAAACCATGATGTCAGTAGTG
GCCGACAAGACGGGCTACCCCACTGAAATGCTGGAGCT
CAGCATGGATATGGAAGCCGATTTAGGTATCGATTCTAT
CAAGCGCGTTGAAATCCTAGGCACAGTACAAGACGAAC
TGCCGGGTTTACCTGAGCTAAATCCTGAAGATCTGGCCG
AGTGTCGCACCTTAGGTGAAATCGTTATGCTCTTTTCG
CAAGATTTAGGGCAAGGCAAGCTAGACCAGAATAATGCG
GCGAATGTGGTAAACGTCGCTGTCGCAGTTGAACCGAC
TATCGATTTGCTACCCCATGATTTACCACAACATGATTT
ACCACCACACAGTGAGGTGGTGCTAAAAAAGTTGCCAG
CGGCGGCTGAGCTAACGCAACTATCGCCACAGCAATCGT
CAAAGCAATCAGCGCAACAAGCTCAAACACGCGTTTTT
GCTAAAGATGCCTGCATTATTATTAGCGATGATGGTCAC
AATGCTGGCGTACTTGCCGAGAAATTGCATGCTCAAGG
TCTAACGGTTGCGGTTGTGCGCTCGCCAGAAAGCCTTGT
AGCCAGTGCATCACCGCTCAATAGCCACATTGCCAGCT
TCACATTGGCGGCGATTGACGATATCAGCATCAGCGTAG
TAATTAATGAGATCAAAACCTTAGGTCAAGTTGCAGGC
TTTATTCATCTGCAACCACAACATAAAACCTCAGCCGAT
GCGAAAGGTTTAGTGTTGTCCAGTGCCGCCAAAGCGTC
GGTCGAGCAAGCGTTCTTGTTCGCCAAGCACTTACAGCC
GCTTTTAACAACAGCAGCAATCGCCAATACTGGCAGCA
GCTTTATCAGCGTCAGTCGTATCGACGGTGGTTTTGGTT
ATCTTAACCACAGCCAAATTGCCCGCAGCGAGTTAAAC
CAAGCGGCATTGGCAGGTCTGACTAAAACCTTGAGCCAC
GAGTGGCCAAGCGTGCATTGCCGCGCCTTAGATATCGC
GCCAGCACTTGATGCCAAGCAACTGGCCAATGCGGTCAT
AGCTGAGTTATTCGCAACCGATAAGCTTTTAGAAGTCG
GAGTGAGTGAAAGTGGTGTGAGTGAAGCTGGCGCAACTG
AAACGCTGGCACGCGTGACCTTAGTTGCAGGCAAGGCA
GATACCCGCCACGGCGCAGCTAACTTGACCAGTGCTGAT
AAAATTCTAGTCACTGGCGGCGCAAAAGGCGTGACCTT
TGAATGTGCCTTGAGTCTGGCAAAACGCAGCAAGGCGCA
CTTTATCCTTGCCGGCAGAAGCAGCCAACAAACCATTC
CCGCTTGGGCACAAGGTAAAAATAACAGCGAACTTAAAG
CCGCAGCTATTGCCCATATTCAAAACTTGGGCGAGAAA
CCTACACCAAAACAAGTAGATGCCTTAGTATGGCCAGTT
CAAAGCAGCCTAGAAATTGCAGCCGCACTAGAAGCCTT
TACTGCAGTAGGCGCGAGTGCCGAGTATTTAAGTCTCGA
TGTAAACAATCCAGACGCGATCGCCAGCACGATAGTGC
CGATCACCGAGTTATCACCTATTACCGGCATCATCCATG
GTGCGGGCGTACTCGCCGATAAACACATCCAAGACAAA
ACCTTAGATGAGTTAGCACGGGTGTATGGCACTAAGGTC
ACAGGCATTAGCAATCTGCTTGCCGCACTGGATTTAGA
TAAAGTAAAACTGATTGCCCTGTTCTCTTCGGCGGCGGG
TTTTTACGGTAATACCGGCCAAAGCGATTACGCCATGT
CCAACGACATACTCAACAAAGCCGCACTGCAACTGGCGC
AGCAACTGCCAAATGCCAAAGTGATGAGCTTCGATTGG
GGTCCGTGGGACGGCGGCATGGTGAATCCTGCGCTGAAA
AAGATGTTTATGGATCGCGGTGTTTATGTAATCCCGCT
AAAAGCAGGTGCTGAGTTATTTGCGAGCCAATTACTGAG
CAACACAGGCGCACAATTGCTGGTCGGTACTGACATGC
AAGGCAGCGCACCTCATGACGACACGCCTAACGAAGTAC
AAGAAACTGAAGGTAGTAATCTAAAAAAGCCTGAAGCG
GATCTGACCACTGACTCGTCGGATCCGCATGCTTTGCTT
AACGCTTTAAATCCAAGCGCAGTAAATATAAGCGCTGT
AAAACTGCAGCGCACACTGGACCCTAAGGCAATGATCTT
TATTGAAGATCACTGCATTAACGGTAATCCCGTATTAC
CGACTGTGTGCGCGATTCAGTGGATGCGTGAAGCCGCCT
TTGATGTGCTCAAGCAACCAGTCAAAGTGCAAAGCTAC
AAGCTGTTGAAGGGCATTATTTTCGACGCTATGACGTTA
GAAAACGGCGCGCCCATCACGCTAGAACTTGAGCTTGC
ACCGATTGCGTTAACGGATAAAGCCGCGAAAGACACAGA
CGAGTACTTGAGTGGACAATTCAGTGCCTTAATCAGCT
TTGAAGGTCGTCCGCAATATCAAGCCATCTTAGTGATTG
ATGATGCTCCTAGTGATAACTTAGCCACTAATAGCAAA
GCAACTGCCTTTGACGCGCATAGCTTGGCAGGATTTTCT
GCCATCACAACCGCAAGCAGCCTTTACAGCGACGGCAC
GCTTTTCCACGGCCCAAGACTGCAAGGTATTGAGTCTGT
GGTGAAGTTTGACGATGCGAGCTTAGTCGCCAAGGTGA
GTCTTCCCCATGTTGCGTTAGCGGACTGCGGGAGTTTTG
TGCCCAATCTGGCCCCTAAAGGCTCACAAGCTTTTGCA
GAAGATTTGTTGCTGCAGGCGATGCTGGTATGGGCAAGA
CTCAAATATGGCGCGGCGAGTTTGCCATCAAGCATTGG
TGAGTTTATCTCCCATGCGCCGTTTGCCTTTGGCGATAC
AGGGTATCTAGTGCTCGAGGTGGTAAAACACTCTGGCC
GTGCCCTTGAAGCCAATATTGCACTTTATCATCAAGATG
GCCGCTTGAGCTGCGAGATGAACAACGCCAAAGTCACC
ATCAGCAAAAACCTTAACGGTGCATTCTTAGCGAATAAA
GTTGCTAATAAAAGCATTGAAAGCGTGGAGGCAAAAGT
CGAGTGAACTTAGCCTATCCTTTAGCAATGACCCATAAC
GGCCAAGCTGAAACGTCGATAGCTGACAAGGCTATTGC
TGACAAAAGCGCCAAGCCACTGCGTATTGCAGTGTTGCT
TGGCGATGCGGTCAACCTTGACTCTCACTCAGCCCAAG
TATTAGGGACGTTTACTGAACGTGAAAGAGTTCAAATTT
GCGCTGCCGATGCCAACCAATCGACAGTACATAGGCCG
ACAGCTCATGAACCCAAAGAGCATGAACCGTCAGTGTAT
GAGCAAAGGTCGCTCACAGCCTTGTTAGGCCAAGCAAC
AACAGCCATTGAGCAAGGCAAGCTCGTTGAACTTAAATT
TGAGGATGGTAATCTACCCCAGTATCTGTATTTGCTCG
ATGGTTTACGCGCCGCCAAACTGCGTCTTCACGCCCACG
CGTTTATTGCTGGCTTTGCCGCTGGCAATGAGGTTACA
GATGTTGCAAATGCGGCGACTGTTGCAAATAAGGCGTTA
GCGGCAGCAAAGCGCAGCCCAGCTCAAACCGTTCAACA
TCAGACTGTCGCCAACACACTCAACGAGGCATTTGTTGC
GCTTCGCCAAGGCGTAACCGCCCTCGCCGCGCGAACAC
AAGCGCCGCTTAAAGGCACTACTGGTATAAAACAGACAA
ACGACACCAATCATCAAACTGGCTATTGGTTTAGCGAC
CAACATCAAGCGCGGGTGTTGTGTCTAAATCTTGTAGCA
AAGACGTCACATCAAGCGGATGAGTCTCGAAATCTAAG
CCAAAGCCTAGTGCTGACCCAAGGCACACAACTCGCCGC
GCCCAAAGCCCTTGTCGATGAAAACCGGCTGTTTGTGC
CGATAAGTAGTGACAGTATTAATGAGTTAAAAGCAAAGC
TGTTTCAGTTGCTTAGTTCACTAGACATTGGCGCACCA
GACACGTCATCTGCATCACATCAACTCACATTCTGGTTT
GAACGCTACGATGCCAACGCACCACTGGCATTAGTCTT
GATGGCGGCATCAAGTGATGATCTCAAACTTGAAGCCAA
AGCCATGCTTACGGCGCTTGAAAATGATGCTATTCGTC
ATCATGGTCAACACTTTAAGACACCAGCGGGTAGCTGCT
TTACCGCTAAGCCGCTAGGGGATGCGGGACTGACTTTT
GTGTATCCTGGCGTTGGCACGGTTTACGCCAATATGTTC
AATAACTTGCATGAGTATTTCCCCGCGCTGTATCACCA
GTTAGAACGTGAAGGCGATTTAAGCGCCATGCTGCAATC
ACCGCAGATTTATGCGGCAGATGTTAAAACCGCGGCTG
GCATGAACCTAAGTCAGCAAGCGATTAGTGGCGTGGGGG
CCAGTTATCTATTCACTAAACTGTTGACCCAAGTCTTT
AATATTAAGCCTAAAATGGCGCTCGGTTACTCAATGGGC
GAAGCAGCCATGTGGGCCAGCCTAGATGTGTGGCAAAC
ACCGCACGCTATGATTAACGCCACTGAAAATAGCGATAT
TTTCAACCATGCGATTTCTGGTGAGCTAACTGCTGTGC
GCCGAGCATGGCAGCTTGCGGATGATGAAGCCATAGTGT
GGAACAGCTTTGTGGTGCGCGCTGATAGCCATGAAATC
AAGGCATTATTGCCAGAGTTTCCTCGCGCCTACTTAGCC
ATCACCCAAGGTGATACTTGTGTCATTGCAGGCTGCGA
AACAAGCTGTAAAGCCCTGCTTGCCACGTTAGGCAAACG
CGGGATTGCCGCCAATCGCGTCACGGCAATGCATACCG
CGCCTGCCCTGTTAGTCCATGGGCAAGTACAAGATTTCT
ATACTCAAGCGCTAAAACCTGAGGCACTGGAACCTGAT
GCGCTGAAAGCGGCAGCGCAAGATTCGTCTGTTCGCTTT
ATTAGCGCAGCGCAAACTGCGCCAGTAATCGTGGATAG
CCACAGCATTGGCCGCGCGATTGCCGATACCTTTTGTTC
GCCACTCGACTTTAGTGCGCTCATTCAAAATGCCACTG
AGCAAGGCGCAAGGCTGTTTGTCGAAGTGGGCGCCGATA
GGCAAACCAGTACACTCATAGATAAAATCAGCCATGCC
CACGCAAGCCAAAGCTCTGCGAACGCGGCGACAGCCGCC
ATTGCCTGCAATGCCAAAGGTGCCGACGCGATCACCAG
CTTACTCAAGTGTTTAGCTCAGCTAATAAGCCACAGAGT
GCCGCTTTCGCTCACGCCGCTTATTCAGCCATTAAGTG
CTAACGCAGCCCCTTTATCATCAGCAGTATCACCAAAAG
GAGAACCCCAGTGAGTTCTCAGCATTCCCCTACGATTG
ATAAAACTACCGTGCCTACCATTGCGTCAAACCGCGCAT
CAAAAAGCGCGTCAAAAATTGCGATCGTCGGCCTCGCG
ACTCAGTATCCTGACGCCGATAATCCTCAAACCTTTTGG
CAAAATCTGCTGGATAAAAAAGACTCTCGCAGTCAAAT
TAGCCGCGAGAAGCTCAATGCCAATCCCGCCGATTACCA
AGGTGTGCAGGGTCAATCTGACCGTTTTTACTGTGATA
AAGGCGGCTACATCCAACACTTCCAGTTTGATGCCAAGG
GTTATCAACTGCCTGAGTCCGCCTTTGACGGTTTAGAT
GAAAGCTTTTTATGGGCACTCGATTGCAGTCGCAAAGCC
CTCCAAGACGCAGGGATTGCCCCAAGCGATGCCGTACT
GGCGCGCACAGGTATCGTGATGGGAACCTTGTCGTTCCC
CACAGCCCGCTCCAACGAATTATTTTTGCCTCTGTATC
ATCAAACCGTTGAAAAGGCGCTGCAAAACAAACTGAATC
AAAGCACTTTTCAGCTGGCTGATTTTAACCAAGCTCAC
GCCGACAAAGCATTAAACGTCGAGCAAGCATTAAACGTC
GCCAATGGCGCCGTTGCCCACACAGCTTCAAAGCTAGT
CAGCGATGCACTCGGTTTAGGTGGCACTCAGTTAAGCCT
GGACGCTGCCTGCGCTAGCTCGGTTTACGCACTTAAAC
TCGCCTGCGATTACCTGACCACAGGCAAGGCCGATATGA
TGCTTGCTGGCGCGGTATCGGGCGCAGATCCCTTCTTT
ATCAACATGGGATTCTCGATTTTTCACGCCTATCCTGAT
CATGGAATCTCAGCACCCTTTGATAGCAACAGCAAGGG
CTTATTCGCCGGAGAAGGCGCAGGGGTGTTAGTATTAAA
ACGCTTAGAAGATGCCGAGCGCGATGGCGATAACATCT
ATGCCGTCGTCAGCGGCATTGGTTTGTCGAACGACGGTA
AAGGCCAGTTTGTATTAAGCCCCAACAGCAAGGGCCAA
GTGCAAGCTTTCGAGCGCGCCTATAGTGCGGCAAACACA
CTGCCCGCCAATATCGAAGTGATTGAATGCCACGCCAC
CGGCACGCCGCTTGGGGATAAGGTCGAACTCGCCTCGAT
GGAACGTTTCTTCGAGGACAAACTCGCGGGCTCTGCAG
TGCCGCTGATCGGTTCGGCAAAATCCAACTTAGGCCATT
TGCTCACAGCCGCAGGCATGCCGGGGATCATGAAGATG
ATTTTTGCCATGCGCTCGGGTCGACTGCCGCCAAGTATT
AACTTATCAGCGCCGATATCCTCGCCTAAGGGCTTGTT
TAGCGAAAAGAATCTGCCAACAGAATTACATGCTTGGCC
CGATAAAGCCGGAAACTCCCGCCGCCACGCCGGTGTTT
CCGTGTTTGGTTTTGGCGGCTGTAACGCGCATTTGTTGC
TGGAATCCTATGTTGCCAATACAAACAAAAAGAATGAA
CAAGCCGCAGCTGCTGTAAGTTATCAGCACACGCCATTA
AATATCATTGGCTTAGCGTCGCACTTCGGCCCTTTATC
CTCCATTAATGCACTGGATAGCACGATTCAAGCTCGGCA
ACATGCCTTTATCCCGCTGCCCGCTAAACGCTGGAAAG
GCTTAGACAAACACCCTGATATTCTGGCCAACGTTGGTT
TAAGCGGAACTGGCCTCGCAGCCGCACCACAGGGCGCG
TATATCGACCAGTTCGATTTTGACTTCCTGCGTTTTAAA
GTGCCGCCCAATGAAGATGACCGCCTGATCTCCCAGCA
ACTGCTGTTGATCAAAGTGGCAGATGAAGCGATTCGTGA
TGCCAACCTTAAGCCCGGTGGCAAGGTTGCTGTATTAG
TGGCGATGGAAACTGAACTCGAATTACATCAGTTCCGTG
GCCGCGTAAATCTGCACACGCAACTGGCAGACAGTCTT
AAAAAGCAAGGGATCACCCTCACACAAGCCGAGTATCTC
GCCCTTGAAAAAATCGCCATGGACAGTGTGCTCGATGC
CGCCAAGCTGAACCAATACACCAGTTTTATTGGCAACAT
CATGGCGTCACGCATCGCATCCCTTTGGGACTTTAATG
GCCCCGCCTTTACCATTTCGGCGGCGGAACAATCGGTCG
CCCGCTGTATCGATGTGGCCGAAAACCTCTTGTCCCAA
GAATCCTTAGATGCCGTAGTGATCGCCGCCGTCGATTTA
AGTGGCAGTCTAGAGCAAGTGATCCTCAAAAACGCCGT
ATCACCAGTGGCATTTAATGCCACTGACACTGGTTGGAA
AGTCGGTGAAGGTGCAGGCGCACTGGTGCTAACTGCTG
AAAATTCAAATACTAATGCTCTACTTAATAATGCCAATA
GCAACAGCTATGGTCACATCAGCGGCCAAGTATTTGGC
GCGATTTGTGACATGCAAGGTAACAGCAACACAGCGCGT
ATTTGCGATGACTTACTAACCCAAGCCAAGGTGAATAG
CAGCCAGATTAGCTTGATTGAAACCAGTATTGCGGTTGA
GCAACTTGCCGATTCAGAGCTGGTACTCAATACCCTGC
TGCCGAGTGTGAACCAGCGCAGCCAAGCCGCTGATACCC
TAGGCCACAATTTGCCGCAGCGGGAATGGCGAGTATTT
TGAGCGCCCTGCTTCAGCTTAAAAATCAAGGGCAATTAA
AAAACCAAGCACAGCAACAAGCTAATCAAGTGCAGCAC
GCGCTCGTTGCCACGTTTAGCCAAGGTAAATGCTCGCAG
TTATTGCTCAGTCAAAGTGCGACGCAAGCACACAGTTT
GCAGCAAAGGCTTGAACAAGACTTAACGCTTTCTGAGCA
AAAACACTTAATTAAACAAGTGACACTTGGTGGCCGCG
ATATCTATCAGCATATCCTTGATACGCCGTTGGCGGACA
TAGATGCAATAAAGCAAAAAGCCCAAGCCATCACTGCA
TTGCCAAAGCGCAGCCAACGCAAACATTTGGCCCAAATA
GCGAGCAAAGACACGAGTGGCTTTGCAACAAGCAGCCC
AACCACGGCTCTACAAAAAGAGACATTAAGCAGCATGCC
AATTAATGCCCTAAGCACGCCCAATGACAACGCAGCTC
AAACAGAGCTAAAAGACGCTGCATTTATACGTAATCAGC
AACTCGCCCGTGAAGCCCATTTAGCCTTTTTACAAAGC
CGTGCGCAGGGCCTGAAACTGGCCGATGCCTTGATGAAA
GCCCAGCTTGCCAGCGAGTTAGCCGTCAATGGCCAAGC
GACGCCGGTACAACAGCAAGCCACTGTTCAGGCGCCAGT
GTATGCATCTGCTCATACACCTGAGCTAGCTCCAGTTG
TTAACTCAGAGGCAAACCCAGCTGCGCTTTATCCAAACC
ATGCAAAAGTGCCTCTGTACACGCCGCCAACACCGATA
AGCAAGCCTTGCATTTGGGATTATGCGGATTTAGTCGAG
TACGCCGAAGGCGATATTGCTAACGTCTTTGGCCAAGA
TTACGCCATTATCGACAGCTATTCGCGCCGTGTACGCCT
GCCAACGACTGACTATTTGCTGGTATCTCGGGTAACGA
AATTAAACGCCCAGATGAACCAATATCAGCCTTGCACTA
TGACCACAGAATACGACATTCCTGTGGACGCGCCGTAT
TTGGTCGATGGCCAAATTCCTTGGGCCGTAGCGGTTGAA
TCGGGTCAATGCGACTTGATGCTGATCAGCTACTTAGG
TATCGACTTTGAAAACAAGGGCGAGCGCGTTTATCGCCT
GCTCGATTGCACCCTGACCTTCCTTGGCGACTTACCGC
GCGGTGGCGACACCCTGCGCTACGATATTTCAATCAATC
ACTTTGCCCGCAATGGCGATACCTTGTTGTTCTTCTTC
TCCTACGAATGTTTCGTGGGCGACAAGCTGATCCTGAAA
ATGGATGGCGGCTGTGCCGGCTTCTTCACCGATAAAGA
ACTAGCCGACGGCAAAGGCGTTATTCACACCGAAGCCGA
AATCAAAGCGCGCAACCTCGCCTTGAACAATCCGAATA
AGCCGCGCTTTAATCCGTTACTCAACTGCGCGCAAAACC
AATTTGATTACAGCCAAATCCATAAACTGCTCGGCGCC
GATATCGGTGGCTGTTTTGGCGGCGCACACGCGGCGCAT
CAAGCCCAATATGGTTTGCAGCCCTCTTTATGTTTTGC
ATCTGAAAAATTCCTGATGATTGAACAAGTCAGCAATCT
TGAGGTGCATGGCGGCGCGTGGGGCTTAGGCTCAGTTC
AAGGCCATAAGCAGCTCGAAGCCGATCATTGGTATTTCC
CGTGTCATTTCAAGGGCGACCAAGTGATGGCGGGGTCG
TTAATGGCCGAAGGCTGTGGTCAATTACTGCAATTCTTT
ATGCTACATATTGGTATGCACCTCGGTGTTAAAGATGG
TCGTTTCCAACCGCTCGAAAACGCGTCACAAAAAGTGCG
TTGTCGCGGTCAAGTGTTGCCGCAATCAGGCCTGCTCA
CCTATCGTATGGAAATCACTGAAATCGGTATGAGCCCGC
GCCCGTATGCTAAGGCGAATATCGATATTCTGCTCAAT
GGTAAAGTGGTTGTGGACTTCCAAAACCTTGGGGTGATG
ATCAAAGAAGAAGCCGAATGCACCCGCTACCTTGCGGA
TAATGATGCCAGCACAGCTGACAATACGACTAAAAATGC
TGCCAAAAATGCTGCTTCGGCTGTGCCGCTAGTTTCGA
CAACACCCGCATCGTTCGCCGCGCCGTTGATGGCCCAGC
TGCCAGATTTAACTGCGCCAACCAATAAAGGCGTAGTG
CCGCTTAAGCATGTGCCTGCGCCGATTGCGCAAACGGAT
TCAAAGTACGCCAACCGCGTGCCCGATACCCTGCCGTT
CACGCCGTACCACATGTTCGAATTTGCCACGGGCGATAT
CGAAAACTGCTTCGGCCCCGATTTCAGCATCTATCGCG
GCCTTATTCCACCGCGCACGCCTTGCGGTGATTTACAGC
TTACCACCCGCGTCATTGCGATTGACGGCAAACGCGGC
GAGCTGAAAAAGCCTTCTTCGTGTATCGCCGAATACGAA
GTGCCCGCAAACGCTTGGTATTACGATAAAAACAGTCA
TCATGCTGTGATGCCCTATTCAGTGCTAATGGAAATATC
ACTGCAGCCAAATGGCTTTATTTCAGGCTATATGGGCA
CCACCTTGGGCTTCCCCGGCCAAGAGCTGTTTTTCCGTA
ACTTAGACGGCAGCGGTAAGCTGCTGCGCCACGTGGAT
TTACGCGGCAAAACCATAGTGAACGACTCACGTTTGTTA
TCGACTGTGATTGCCGGCAGCAACATCATCCAGAATTT
CAGCTTCGAGTTAAGCTGCGATGGCGAGCCTTTCTACCA
AGGTAAAGCGGTATTTGGTTACTTCAAGGGCGATGCGC
TGAAAAACCAACTCGGCATAGACAACGGCAAGATCACAC
AGCCTTGGCATGTGCAAAATGGCATAGCCGCCGATAGC
CAAATCAATCTGTTAGATAAACAGCATCGCAGCTTTAAC
GCGCCAGAAGGTCAGCCGCATTACCGTTTAGCGGGCGG
TCAGCTTAACTTTATCGACAAGGCCGACATAGTGAAAGC
CGGCGGTAAAGCGGGCCTTGGCTATTTATACGCCGAGC
GCACCATTGACCCGAGTGATTGGTTCTTCCAATTCCACT
TCCATCAAGATCCGGTAATGCCAGGCTCATTAGGGGTT
GAAGCGATTATCGAGCTGATGCAAACCTATGCGATTGAC
CAAGACCTTGGTGCGGGCTTTAAGAGTCCAAAATTCGG
CCAGATATTATCGGATATCAAATGGAAGTATCGCGGCCA
AATCAACCCATTAAACAAACAGATGTCGCTGGATGTGC
ACATTACCAGCGTGACAGACGACAATGGCAAACGCATCA
TTATGGGCGATGCCAACTTGAGTAAAGATGGTCTGCGA
ATTTATGAAGTCAAAGATATCGCCATCTGTATTGAAGAA
GCTTAATCACCTTGCTCATTTAGAAAGACGCTAATTAC
AAATACTGTGTCGCCTTGGCCAGAATGCCTAAGGCGGCA
ATAAAAAGAGAATACATATGACAAGCCATACTCTCGAT
CAATTTAATAGTAATAACGAAAAACTCAGCCCTTGGCCG
TGGCAAGTCAACGATGCCGCGCTGAGCTTTGATATCGA
CTCATTAGGCAAAAAACTCAAAGATTTAAGCCAAGCCTG
TTACTTAGTGAATCACAGTGAAAAAGGCTTAGGCATAG
CGCAAACAGCCGAAGTAACCACAAGCGACAGCCAAGCGC
CACTAGGCTCACACCCCGTCAGCGCCTTTGCGCCCGCC
CTTGGCACCCAAAGTTTAGGCGACAGTAATTTTCGCCGC
GTACACGGGGTTAAATACGCTTACTACGCTGGCGCTAT
GGCTAACGGTATTGCCTCAGAAGAACTGGTTATCGCGCT
GGGCCAAGCGGGCATTTTGTGTTCGTTTGGCGCGGCGG
GGTTAATCCCATCCCGCGTTGAAGCGGCCATTACTCGCA
TTCAAGCGGCGCTGCCTAATGGTCCTTACGCCTTTAAT
TTAATTCACAGCCCAAGCGAGCCCGCATTAGAGCGCGGC
AGTGTTGAGTTGTTCTTAAAACATAAAGTGCGCACGGT
CGAAGCCTCGGCATTTTTAGGTTTAACGCCACAAATCGT
CTATTACCGCGCAGCAGGTTTGAGCCGCGACGCACATG
GCGACATCGTCATTGGCAACAAAGTCATAGCCAAAATCA
GTCGCACCGAAGTCGCGACTAAGTTTATGGAGCCGGCG
CCTGCCAAAATTCTGCAGCAATTAGTCAGTGAAGGCCTT
ATCAGCCAAGATCAAATGGCGATGGCGCAACTTGTACC
CATGGCGGACGATATCACGGCCGAAGCCGATTCTGGCGG
CCATACCGACAATCGTCCACTGGTCACGCTATTGCCGA
CGATTTTGGCGCTCAAAGATGAAATCCAAGCTAAGTATC
AATACAAGACGCCCATCCGTGTGGGAGCAGGCGGCGGC
GTTGGTACCCCCGACGCAGCATTAGCCACCTTCAACATG
GGCGCGGCCTTTATCGTCACAGGTTCAATCAACCAAGC
GTGTGTGGAGGCGGGCGCGAGCGAACACACACGTAAGTT
ACTCGCCACCACAGAAATGGCCGATGTGACTATGGCAC
CCGCCGCCGATATGTTCGAAATGGGCGTGAAATTACAAG
TGGTTAAGCGTGGCACTCTGTTCCCGATGCGCGCCAAT
AAGCTTTATGAAATCTACACCCGTTACGATTCAATTGAT
GCCATCCCCGCGGACGAGCGTAAAAAGCTCGAAGAGCA
AGTGTTTCGCGCATCACTCGATGACATTTGGGCGGGCAC
TGTCGCCCACTTTAACGAGCGCGATCCTAAGCAAATCG
AGCGCGCACTGGATAACCCCAAACGTAAGATGGCGTTGA
TTTTCCGCTGGTATTTGGGTCTGTCTAGCCGCTGGTCG
AACACAGGTGAAGTCGGCCGCGAAATGGATTACCAAATT
TGGGCAGGCCCTGCCCTTGGCGCATTTAATGCGTGGGC
AAAAGGCAGTTATTTAGATGACTATAAAGCCCGTAATGC
GGTCGATTTAGCCAAACATTTAATGGTGGGCGCGGCCT
ATCAATCCCGGATTAACTTGCTGTTATCCCAAGGGGTTA
GCATTCCAGTTAGCCTGCAACGCTGGAAACCGCTAAAT
CGTTTTTAACCCCTAACGGACTATCGAGACATCAAGTAG
GGACTCAACTCCCTACTTGATGCAATCTTCTTCACTAC
CTCGTTAAATCGAAGCCGCCGACCTTATTACCAATCTAA
TCCCTTGTTTGTGCCAATGCCGCTAAGCACTGTCGCTC
CAGTGGCACGCCGTAAACCCATCCTTGGGGGCTCAACGG
AAAAGTCCCTTTTTCCGATGG (SEQ ID NO: 1)
pfaA gene sequence SEQ ID NO: 2
ATGAGCCATACCCCTTCTGTACCTAATTCTGCGACTGAG
TCAAAAAAAGATAAACGACTCAACAAACGTTTGAAAGA
TATGCCCGTTGCCATCGTCGGCATGGCCAGTATCTTCGC
CAACTCGCGTTATTTAAATAAATTTTGGGATTTGATCA
GCGAAAAAATCGATGCCATTACTGACATCCCAGACACCC
ATTGGCGCGCCGAAGATTACTACGATGCAGACAAAAGC
AAAGCCGATAAAAGCTACTGTAAACGCGGTGGTTTTTTG
CCTGAAGTCGACTTCAACCCAATGGAATTTGGTCTGCC
GCCCAATATTCTTGAGCTAACAGATACCTCGCAACTGCT
GTCGCTGATTGTCGCCAAAGAAGTGCTCGCCGATGCCA
ATCTGCCCACCGATTATGATCGCGATCGTATCGGCATCA
CGCTAGGCGTGGGCGGTGGTCAAAAAATCAGTCAGAGC
TTGAACTCGCGCCTGCAATATCCAGTATTGAAAAAAGTA
TTCAAAAGCAGCGGCCTGAGCGATGAAGACAGCGAAAT
GCTGATCAAAAAGTTCCAAGACCAATATATCCACTGGGA
AGAAAACTCTTTCCCAGGGTCCTTAGGCAACGTGATTG
CAGGCCGTATCGCCAACCGTTTTGATTTTGGCGGCATGA
ACTGTGTGGTCGATGCTGCCTGCGCGGGCTCACTTGCC
GCTATGCGTATGGCGTTGACTGAACTGACCGAAGGTCGC
AGCGACATGATGATCACCGGCGGCGTCTGTACCGACAA
CTCGCCGTCCATGTATATGAGCTTCTCAAAAACGCCTGC
CTTCACTACCAATGAAACCATTCAACCCTTTGATATCG
ATTCAAAGGGCATGATGATCGGCGAAGGTATTGGCATGG
TAGCACTTAAGCGCCTTGAAGATGCCGAGCGCGATGGC
GACCGGATTTATGCCGTCATCAAAGGCGTTGGCGCCTCA
TCGGACGGTAAATTTAAGAGTATTTATGCGCCGCGCCC
TGAAGGCCAAGCTAAAGCATTGGAGCGCGCCTACGACGA
CGCGGGTTTTGCCCCGCACAGCATTGGCTTAGTTGAAG
CCCATGGCACGGGCACTGCCGCAGGTGATGTGGCCGAAT
TCAATGGCTTAAAATCGGTATTTGCCCAAGGCAACGAC
ACCAATCAACATATCGCGTTAGGTTCAGTGAAATCCCAA
GTGGGCCACACTAAATCAACCGCAGGTACTGCTGGGGT
AATCAAAGCCGCGCTGGCGCTGCACCACAAGGTATTACC
TGCGACCATTAACGTCAGCAAGCCTAATCCAAAACTGA
ATATCGAAAGCTCACCATTCTATTTAAATACCGAAACGC
GCCCTTGGCTGCAACGCACTGACGGTACGCCGCGCCGT
GCTGGCATAAGTTCCTTTGGTTTTGGCGGCACTAACTTC
CATCTCGTATTAGAAGAATACAAACCCGAGCACAGCCG
TGACGAGCAATATCGTCAGCGCAGTGTGCCGCAAACGCT
ATTATTTGCCGCCGCCAATAAAGCCGCGCTGCTTAGCG
AGTTAAAAGCTGCGCTGAGCCAAAGCTTGAATACGAACG
CGAATAAGAGCAGCGCTGCGAGCCTGAACGCTATCGCT
CAGCAATATCCGCTACGCGCGCTGGCAGACACAGATGCC
CGTTTAGGTTTTGTGGCTAAGGATATCGCTCAGCTGCA
AGCTCAGTTGAATCAAGCCATCTCTCATCTAGAAAGCAG
TGCGCATTTAGAAAGCGGGCTCCTAGATACCAGTGCCA
GCGAGGCGTGGCAGTTACCTTCTGGGATCAACTACCGCG
CAAAAGCTTTAGTTGCTAAAGATGAATCAAAGAAAGTG
GCCGCTCTGTTTGCCGGTCAAGGTTCACAGTACCTGAAC
ATGGGCCGCGAACTCGCCTGCCATTTCCCTGAAATGCG
CCAACAAGTGATGGCCAGCGATAAGGTATTTGCTCACCA
CGGCCAAACGCCGTTATCCAACATCCTTTATCCTATTC
CGGCATTCGATGCCGATGCGGTTAAAGCCCAAGAAGCGG
CGCTGACCAATACCCTGTTCGCCCAAAGCGCCATAGGC
GCGGTTTCAATGGCGCAATATTCACTATTAACTCAGGCA
GGTTTTGCCCCAGATATGGTGGCGGGTCACAGCTTTGG
TGAATTGTCAGCCCTTTGCGCGGCTGGCGTGATTTCGAG
TGTTGATTACGTCGAACTGGCCTTCGCCCGTGGGCACG
CCATGGCGCAAGTGCCAAGTGATACTGACGCTCAAGTCG
ATTTAGGCAAGATGTTTGCCATCATCCTCAAACAAAAG
AGTGATATCGATGCGCTTAATCGCTGTTTAGCTCAGTTT
GATGGCGTTAAAATTGCCAACTACAACGCGCCGACACA
ATTAGTCATCGCAGGTGGCACAGAGCAAACTCAGCTGGC
TGCCAAAGCTATTAGCGAACAGGGCTTTAAAGCGATTG
CCCTGCCCGTCTCTGGCGCGTTCCACACCCCCTTGGTTG
GGCACGCACAAAAGCCATTTGCCAAGGCCATTGATAAA
GCTAAATTCAGCGCGCCTTCAATCGCACTTTATGCCAAC
GGCACAGGTCAATTGCACCCAAGCGATGGCAAAGCGAT
TAAAGCAGAATTCAAACAACACATGCTGCAATCGGTTCG
TTTTAGCGAACAACTGCAGGCCATGTATGATGCTGGCG
CCCGTGTGTTTGTTGAGTTTGGCCCTAAGAACATACTAC
AAAAGCTCGTTGAAAATACCTTAAGTGAACACTTAAAT
GAGCTTTGCCTTGTCAGCATGAACCCGAACCCTAAGGGC
GACAGTGACAGTCAATTACGCTTAGCCGCTGTGCAACT
CGCGGTAGCCGGTGTGGCGTTAACTGAGGTTGACCCCTA
TCAAGCCGTCACGTCACAGGAGATTGCCGAGCGTGAAG
CGCCATCGGCGATGAACATCAAACTCACCGCCACTAACC
ACATCAGTGCCGCAACCCAAGCTAAGATGGCGAAATCT
CTCGCCACAGGCAGCGTGACTAGCCAAGTGCAATATGTG
GATCGCATCGTTGAAACGATTCTCGAAAAAGAAGTCGA
AAAAATCGTTCAGAAAGAAGTCATAGTCGAAAAAGTGGT
TGAGAAAATAATAGAAGTGGAAGCGAATCAAGTGGCAG
CTGTTGAAATGAAACAAAAACTCCTAAGCGTGACGCAAG
GCTTAAATCACCAACAAGCGACGGCGCAGATGAGCCCA
AGCACAGCAAACGTCAGCGGCGATGCGTTAACGGCATTT
TTCAGCGCACAATCTCAAGCGGCGCAGTTACATCAACA
GTTTTTAGCCATCCCGCAGCAATATGGCGATACTTTCAC
GACACTGATGACAGAACAAGCCAAGATGGCGAGCCTTG
GCATTGCAATCCCAGAAAGCCTGCAACGCTCGATGGAAA
TGTTCCACCAGCATCAAGCGCAAACTCTGCAAAGCCAC
GCCGAATTTATGCAACTGCAAAGCAGTAGCAGCCAAGCG
GCCTTGGCCATGTTAAACAATGCGCCGATTAACTTTAC
CCCAGCGGTTGCAAGTCAGCCACAAGCAACTGTACCTGT
TGCTCCTGCACCTGTTGCTGCATCGACAGTGGCACACA
ATGCCGCACCAGTCGCGGCTCAGGCTGTTGCGACAAGAC
CAGCGGTTAGCACACCCGTTCCTCCTGTTGTGCAAACC
GCTCCCGTTGCTTATGCGCCTGCTGCGACAGTACAAGTT
GCGCCAGCAGCTCCTGCCGCACCCGCATTAGTCATGCC
TGCTGTGGTGATGCCAGAAGTGACACCTGTGGCACCAAC
AATGAACGGTTTAAGTGCTGAAAAAGTACAGCAAACAA
TGATGGCTGTGGTGGCTGGCAAGACAGGCTATCCGACTG
AAATGCTGGAGCTCAGCATGGATATGGAAGCCGATCTC
GGTATCGATTCAATTAAGCGCGTTGAAATCTTAGGCACA
GTGCAAGACGAGCTGCCGAGTTTACCTGAACTCAGCCC
AGAAGATTTGGCTGAGTGCCGTACCTTGGGTGAAATCGT
AAGCTATATGAATAGCAAATTACCTGCAGCGGGAACTA
TTGCTTCGACAGCAGCGCCAGTTGCTCAAACCATTGCCG
CTCCTGCATCTAACGCTTTAAGCGCTGCATTAGTTCAA
CAAACTATGATGGCTGTGGTGGCCGACAAGACAGGCTAC
CCCACTGAAATGCTGGAACTTGGCATGGATATGGAAGC
CGATTTAGGTATCGATTCTATTAAGCGCGTTGAGATTTT
AGGCACAGTGCAAGACGAACTGCCGGGTTTACCCGAAC
TTAGCCCAGAAGATTTGGCCGAATGTCGCACCTTAGGTG
AAATCGTTACCTATATGAACTCTAAACTGCCAGCAGCG
GGTTCAGTTGTAGTTTCTACAACAGCACAAGCCGCGCCT
GCCGACAGCGGTTTAAGCGCTGAGTTAGTGCATTCTTC
TGAAATCGAGAGCACCATGATGGCTGTGGTGGCCGACAA
GACAGGCTATCCGACTGAAATGCTAGAACTGAGCATGG
ATATGGAAGCCGATCTCGGTATCGACTCCATCAAGCGCG
TTGAAATCCTAGGCACAGTGCAAGACGAACTGCCGGGC
TTACCTGAACTTAGCCCAGAAGATTTGGCCGAGTGTCGT
ACCTTGGGTGAAATCGTTAGCTATATGAATAGCAAATT
ACCAGCAGCAGGAGTTATTGCTTCGACAGCTGCGCCAGT
TGCTCAAACCATTGCAGCTCCTGCCGCAATCGGTTTAA
GCGCTGCATTAGTGCAACAAACTATGATGGCTGTGGTGG
CTGACAAGACAGGTTACCCCACTGAAATGCTGGAACTC
AGCATGGATATGGAAGCCGATTTAGGTATCGATTCTATC
AAGCGCGTTGAAATTTTAGGCACAGTGCAAGACGAACT
TCCTGGCTTACCTGAACTCAATCCTGAAGATCTAGCCGA
GTGTCGCACCTTAGGTGAAATCGTTGACTACATGAACA
GCAAACTACCTGCGGTTGGCTCGACTTCAACTATAACAG
CAGCACAGGTCACTGTTACCGCTACAATAAATAACGGC
TTAAGTGCTGAAAAAATCCAACAAACCATGATGTCAGTA
GTGGCCGACAAGACGGGCTACCCCACTGAAATGCTGGA
GCTCAGCATGGATATGGAAGCCGATTTAGGTATCGATTC
TATCAAGCGCGTTGAAATCCTAGGCACAGTACAAGACG
AACTGCCGGGTTTACCTGAGCTAAATCCTGAAGATCTGG
CCGAGTGTCGCACCTTAGGTGAAATCGTTATGCTCTTT
TCGCAAGATTTAGGGCAAGGCAAGCTAGACCAGAATAAT
GCGGCGAATGTGGTAAACGTCGCTGTCGCAGTTGAACC
GACTATCGATTTGCTACCCCATGATTTACCACAACATGA
TTTACCACCACACAGTGAGGTGGTGCTAAAAAAGTTGC
CAGCGGCGGCTGAGCTAACGCAACTATCGCCACAGCAAT
CGTCAAAGCAATCAGCGCAACAAGCTCAAACACGCGTT
TTTGCTAAAGATGCCTGCATTATTATTAGCGATGATGGT
CACAATGCTGGCGTACTTGCCGAGAAATTGCATGCTCA
AGGTCTAACGGTTGCGGTTGTGCGCTCGCCAGAAAGCCT
TGTAGCCAGTGCATCACCGCTCAATAGCCACATTGCCA
GCTTCACATTGGCGGCGATTGACGATATCAGCATCAGCG
TAGTAATTAATGAGATCAAAACCTTAGGTCAAGTTGCA
GGCTTTATTCATCTGCAACCACAACATAAAACCTCAGCC
GATGCGAAAGGTTTAGTGTTGTCCAGTGCCGCCAAAGC
GTCGGTCGAGCAAGCGTTCTTGTTCGCCAAGCACTTACA
GCCGCTTTTAACAACAGCAGCAATCGCCAATACTGGCA
GCAGCTTTATCAGCGTCAGTCGTATCGACGGTGGTTTTG
GTTATCTTAACCACAGCCAAATTGCCCGCAGCGAGTTA
AACCAAGCGGCATTGGCAGGTCTGACTAAAACCTTGAGC
CACGAGTGGCCAAGCGTGCATTGCCGCGCCTTAGATAT
CGCGCCAGCACTTGATGCCAAGCAACTGGCCAATGCGGT
CATAGCTGAGTTATTCGCAACCGATAAGCTTTTAGAAG
TCGGAGTGAGTGAAAGTGGTGTGAGTGAAGCTGGCGCAA
CTGAAACGCTGGCACGCGTGACCTTAGTTGCAGGCAAG
GCAGATACCCGCCACGGCGCAGCTAACTTGACCAGTGCT
GATAAAATTCTAGTCACTGGCGGCGCAAAAGGCGTGAC
CTTTGAATGTGCCTTGAGTCTGGCAAAACGCAGCAAGGC
GCACTTTATCCTTGCCGGCAGAAGCAGCCAACAAACCA
TTCCCGCTTGGGCACAAGGTAAAAATAACAGCGAACTTA
AAGCCGCAGCTATTGCCCATATTCAAAACTTGGGCGAG
AAACCTACACCAAAACAAGTAGATGCCTTAGTATGGCCA
GTTCAAAGCAGCCTAGAAATTGCAGCCGCACTAGAAGC
CTTTACTGCAGTAGGCGCGAGTGCCGAGTATTTAAGTCT
CGATGTAAACAATCCAGACGCGATCGCCAGCACGATAG
TGCCGATCACCGAGTTATCACCTATTACCGGCATCATCC
ATGGTGCGGGCGTACTCGCCGATAAACACATCCAAGAC
AAAACCTTAGATGAGTTAGCACGGGTGTATGGCACTAAG
GTCACAGGCATTAGCAATCTGCTTGCCGCACTGGATTT
AGATAAAGTAAAACTGATTGCCCTGTTCTCTTCGGCGGC
GGGTTTTTACGGTAATACCGGCCAAAGCGATTACGCCA
TGTCCAACGACATACTCAACAAAGCCGCACTGCAACTGG
CGCAGCAACTGCCAAATGCCAAAGTGATGAGCTTCGAT
TGGGGTCCGTGGGACGGCGGCATGGTGAATCCTGCGCTG
AAAAAGATGTTTATGGATCGCGGTGTTTATGTAATCCC
GCTAAAAGCAGGTGCTGAGTTATTTGCGAGCCAATTACT
GAGCAACACAGGCGCACAATTGCTGGTCGGTACTGACA
TGCAAGGCAGCGCACCTCATGACGACACGCCTAACGAAG
TACAAGAAACTGAAGGTAGTAATCTAAAAAAGCCTGAA
GCGGATCTGACCACTGACTCGTCGGATCCGCATGCTTTG
CTTAACGCTTTAAATCCAAGCGCAGTAAATATAAGCGC
TGTAAAACTGCAGCGCACACTGGACCCTAAGGCAATGAT
CTTTATTGAAGATCACTGCATTAACGGTAATCCCGTAT
TACCGACTGTGTGCGCGATTCAGTGGATGCGTGAAGCCG
CCTTTGATGTGCTCAAGCAACCAGTCAAAGTGCAAAGC
TACAAGCTGTTGAAGGGCATTATTTTCGACGCTATGACG
TTAGAAAACGGCGCGCCCATCACGCTAGAACTTGAGCT
TGCACCGATTGCGTTAACGGATAAAGCCGCGAAAGACAC
AGACGAGTACTTGAGTGGACAATTCAGTGCCTTAATCA
GCTTTGAAGGTCGTCCGCAATATCAAGCCATCTTAGTGA
TTGATGATGCTCCTAGTGATAACTTAGCCACTAATAGC
AAAGCAACTGCCTTTGACGCGCATAGCTTGGCAGGATTT
TCTGCCATCACAACCGCAAGCAGCCTTTACAGCGACGG
CACGCTTTTCCACGGCCCAAGACTGCAAGGTATTGAGTC
TGTGGTGAAGTTTGACGATGCGAGCTTAGTCGCCAAGG
TGAGTCTTCCCCATGTTGCGTTAGCGGACTGCGGGAGTT
TTGTGCCCAATCTGGCCCCTAAAGGCTCACAAGCTTTT
GCAGAAGATTTGTTGCTGCAGGCGATGCTGGTATGGGCA
AGACTCAAATATGGCGCGGCGAGTTTGCCATCAAGCAT
TGGTGAGTTTATCTCCCATGCGCCGTTTGCCTTTGGCGA
TACAGGGTATCTAGTGCTCGAGGTGGTAAAACACTCTG
GCCGTGCCCTTGAAGCCAATATTGCACTTTATCATCAAG
ATGGCCGCTTGAGCTGCGAGATGAACAACGCCAAAGTC
ACCATCAGCAAAAACCTTAACGGTGCATTCTTAGCGAAT
AAAGTTGCTAATAAAAGCATTGAAAGCGTGGAGGCAAA
AGTCGAGT (SEQ ID NO: 2)
pfaB gene sequence SEQ ID NO: 3
ATGACCCATAACGGCCAAGCTGAAACGTCGATAGCTGAC
AAGGCTATTGCTGACAAAAGCGCCAAGCCACTGCGTAT
TGCAGTGTTGCTTGGCGATGCGGTCAACCTTGACTCTCA
CTCAGCCCAAGTATTAGGGACGTTTACTGAACGTGAAA
GAGTTCAAATTTGCGCTGCCGATGCCAACCAATCGACAG
TACATAGGCCGACAGCTCATGAACCCAAAGAGCATGAA
CCGTCAGTGTATGAGCAAAGGTCGCTCACAGCCTTGTTA
GGCCAAGCAACAACAGCCATTGAGCAAGGCAAGCTCGT
TGAACTTAAATTTGAGGATGGTAATCTACCCCAGTATCT
GTATTTGCTCGATGGTTTACGCGCCGCCAAACTGCGTC
TTCACGCCCACGCGTTTATTGCTGGCTTTGCCGCTGGCA
ATGAGGTTACAGATGTTGCAAATGCGGCGACTGTTGCA
AATAAGGCGTTAGCGGCAGCAAAGCGCAGCCCAGCTCAA
ACCGTTCAACATCAGACTGTCGCCAACACACTCAACGA
GGCATTTGTTGCGCTTCGCCAAGGCGTAACCGCCCTCGC
CGCGCGAACACAAGCGCCGCTTAAAGGCACTACTGGTA
TAAAACAGACAAACGACACCAATCATCAAACTGGCTATT
GGTTTAGCGACCAACATCAAGCGCGGGTGTTGTGTCTA
AATCTTGTAGCAAAGACGTCACATCAAGCGGATGAGTCT
CGAAATCTAAGCCAAAGCCTAGTGCTGACCCAAGGCAC
ACAACTCGCCGCGCCCAAAGCCCTTGTCGATGAAAACCG
GCTGTTTGTGCCGATAAGTAGTGACAGTATTAATGAGT
TAAAAGCAAAGCTGTTTCAGTTGCTTAGTTCACTAGACA
TTGGCGCACCAGACACGTCATCTGCATCACATCAACTC
ACATTCTGGTTTGAACGCTACGATGCCAACGCACCACTG
GCATTAGTCTTGATGGCGGCATCAAGTGATGATCTCAA
ACTTGAAGCCAAAGCCATGCTTACGGCGCTTGAAAATGA
TGCTATTCGTCATCATGGTCAACACTTTAAGACACCAG
CGGGTAGCTGCTTTACCGCTAAGCCGCTAGGGGATGCGG
GACTGACTTTTGTGTATCCTGGCGTTGGCACGGTTTAC
GCCAATATGTTCAATAACTTGCATGAGTATTTCCCCGCG
CTGTATCACCAGTTAGAACGTGAAGGCGATTTAAGCGC
CATGCTGCAATCACCGCAGATTTATGCGGCAGATGTTAA
AACCGCGGCTGGCATGAACCTAAGTCAGCAAGCGATTA
GTGGCGTGGGGGCCAGTTATCTATTCACTAAACTGTTGA
CCCAAGTCTTTAATATTAAGCCTAAAATGGCGCTCGGT
TACTCAATGGGCGAAGCAGCCATGTGGGCCAGCCTAGAT
GTGTGGCAAACACCGCACGCTATGATTAACGCCACTGA
AAATAGCGATATTTTCAACCATGCGATTTCTGGTGAGCT
AACTGCTGTGCGCCGAGCATGGCAGCTTGCGGATGATG
AAGCCATAGTGTGGAACAGCTTTGTGGTGCGCGCTGATA
GCCATGAAATCAAGGCATTATTGCCAGAGTTTCCTCGC
GCCTACTTAGCCATCACCCAAGGTGATACTTGTGTCATT
GCAGGCTGCGAAACAAGCTGTAAAGCCCTGCTTGCCAC
GTTAGGCAAACGCGGGATTGCCGCCAATCGCGTCACGGC
AATGCATACCGCGCCTGCCCTGTTAGTCCATGGGCAAG
TACAAGATTTCTATACTCAAGCGCTAAAACCTGAGGCAC
TGGAACCTGATGCGCTGAAAGCGGCAGCGCAAGATTCG
TCTGTTCGCTTTATTAGCGCAGCGCAAACTGCGCCAGTA
ATCGTGGATAGCCACAGCATTGGCCGCGCGATTGCCGA
TACCTTTTGTTCGCCACTCGACTTTAGTGCGCTCATTCA
AAATGCCACTGAGCAAGGCGCAAGGCTGTTTGTCGAAG
TGGGCGCCGATAGGCAAACCAGTACACTCATAGATAAAA
TCAGCCATGCCCACGCAAGCCAAAGCTCTGCGAACGCG
GCGACAGCCGCCATTGCCTGCAATGCCAAAGGTGCCGAC
GCGATCACCAGCTTACTCAAGTGTTTAGCTCAGCTAAT
AAGCCACAGAGTGCCGCTTTCGCTCACGCCGCTTATTCA
GCCATTAAGTGCTAACGCAGCCCCTTTATCATCAGCAG
TATCACCAAAAGGAGAACCCCAGT (SEQ ID NO: 3)
pfaC gene sequence SEQ ID NO: 4
ATGGCGAGTATTTTGAGCGCCCTGCTTCAGCTTAAAAAT
CAAGGGCAATTAAAAAACCAAGCACAGCAACAAGCTAA
TCAAGTGCAGCACGCGCTCGTTGCCACGTTTAGCCAAGG
TAAATGCTCGCAGTTATTGCTCAGTCAAAGTGCGACGC
AAGCACACAGTTTGCAGCAAAGGCTTGAACAAGACTTAA
CGCTTTCTGAGCAAAAACACTTAATTAAACAAGTGACA
CTTGGTGGCCGCGATATCTATCAGCATATCCTTGATACG
CCGTTGGCGGACATAGATGCAATAAAGCAAAAAGCCCA
AGCCATCACTGCATTGCCAAAGCGCAGCCAACGCAAACA
TTTGGCCCAAATAGCGAGCAAAGACACGAGTGGCTTTG
CAACAAGCAGCCCAACCACGGCTCTACAAAAAGAGACAT
TAAGCAGCATGCCAATTAATGCCCTAAGCACGCCCAAT
GACAACGCAGCTCAAACAGAGCTAAAAGACGCTGCATTT
ATACGTAATCAGCAACTCGCCCGTGAAGCCCATTTAGC
CTTTTTACAAAGCCGTGCGCAGGGCCTGAAACTGGCCGA
TGCCTTGATGAAAGCCCAGCTTGCCAGCGAGTTAGCCG
TCAATGGCCAAGCGACGCCGGTACAACAGCAAGCCACTG
TTCAGGCGCCAGTGTATGCATCTGCTCATACACCTGAG
CTAGCTCCAGTTGTTAACTCAGAGGCAAACCCAGCTGCG
CTTTATCCAAACCATGCAAAAGTGCCTCTGTACACGCC
GCCAACACCGATAAGCAAGCCTTGCATTTGGGATTATGC
GGATTTAGTCGAGTACGCCGAAGGCGATATTGCTAACG
TCTTTGGCCAAGATTACGCCATTATCGACAGCTATTCGC
GCCGTGTACGCCTGCCAACGACTGACTATTTGCTGGTA
TCTCGGGTAACGAAATTAAACGCCCAGATGAACCAATAT
CAGCCTTGCACTATGACCACAGAATACGACATTCCTGT
GGACGCGCCGTATTTGGTCGATGGCCAAATTCCTTGGGC
CGTAGCGGTTGAATCGGGTCAATGCGACTTGATGCTGA
TCAGCTACTTAGGTATCGACTTTGAAAACAAGGGCGAGC
GCGTTTATCGCCTGCTCGATTGCACCCTGACCTTCCTT
GGCGACTTACCGCGCGGTGGCGACACCCTGCGCTACGAT
ATTTCAATCAATCACTTTGCCCGCAATGGCGATACCTT
GTTGTTCTTCTTCTCCTACGAATGTTTCGTGGGCGACAA
GCTGATCCTGAAAATGGATGGCGGCTGTGCCGGCTTCT
TCACCGATAAAGAACTAGCCGACGGCAAAGGCGTTATTC
ACACCGAAGCCGAAATCAAAGCGCGCAACCTCGCCTTG
AACAATCCGAATAAGCCGCGCTTTAATCCGTTACTCAAC
TGCGCGCAAAACCAATTTGATTACAGCCAAATCCATAA
ACTGCTCGGCGCCGATATCGGTGGCTGTTTTGGCGGCGC
ACACGCGGCGCATCAAGCCCAATATGGTTTGCAGCCCT
CTTTATGTTTTGCATCTGAAAAATTCCTGATGATTGAAC
AAGTCAGCAATCTTGAGGTGCATGGCGGCGCGTGGGGC
TTAGGCTCAGTTCAAGGCCATAAGCAGCTCGAAGCCGAT
CATTGGTATTTCCCGTGTCATTTCAAGGGCGACCAAGT
GATGGCGGGGTCGTTAATGGCCGAAGGCTGTGGTCAATT
ACTGCAATTCTTTATGCTACATATTGGTATGCACCTCG
GTGTTAAAGATGGTCGTTTCCAACCGCTCGAAAACGCGT
CACAAAAAGTGCGTTGTCGCGGTCAAGTGTTGCCGCAA
TCAGGCCTGCTCACCTATCGTATGGAAATCACTGAAATC
GGTATGAGCCCGCGCCCGTATGCTAAGGCGAATATCGA
TATTCTGCTCAATGGTAAAGTGGTTGTGGACTTCCAAAA
CCTTGGGGTGATGATCAAAGAAGAAGCCGAATGCACCC
GCTACCTTGCGGATAATGATGCCAGCACAGCTGACAATA
CGACTAAAAATGCTGCCAAAAATGCTGCTTCGGCTGTG
CCGCTAGTTTCGACAACACCCGCATCGTTCGCCGCGCCG
TTGATGGCCCAGCTGCCAGATTTAACTGCGCCAACCAA
TAAAGGCGTAGTGCCGCTTAAGCATGTGCCTGCGCCGAT
TGCGCAAACGGATTCAAAGTACGCCAACCGCGTGCCCG
ATACCCTGCCGTTCACGCCGTACCACATGTTCGAATTTG
CCACGGGCGATATCGAAAACTGCTTCGGCCCCGATTTC
AGCATCTATCGCGGCCTTATTCCACCGCGCACGCCTTGC
GGTGATTTACAGCTTACCACCCGCGTCATTGCGATTGA
CGGCAAACGCGGCGAGCTGAAAAAGCCTTCTTCGTGTAT
CGCCGAATACGAAGTGCCCGCAAACGCTTGGTATTACG
ATAAAAACAGTCATCATGCTGTGATGCCCTATTCAGTGC
TAATGGAAATATCACTGCAGCCAAATGGCTTTATTTCA
GGCTATATGGGCACCACCTTGGGCTTCCCCGGCCAAGAG
CTGTTTTTCCGTAACTTAGACGGCAGCGGTAAGCTGCT
GCGCCACGTGGATTTACGCGGCAAAACCATAGTGAACGA
CTCACGTTTGTTATCGACTGTGATTGCCGGCAGCAACA
TCATCCAGAATTTCAGCTTCGAGTTAAGCTGCGATGGCG
AGCCTTTCTACCAAGGTAAAGCGGTATTTGGTTACTTC
AAGGGCGATGCGCTGAAAAACCAACTCGGCATAGACAAC
GGCAAGATCACACAGCCTTGGCATGTGCAAAATGGCAT
AGCCGCCGATAGCCAAATCAATCTGTTAGATAAACAGCA
TCGCAGCTTTAACGCGCCAGAAGGTCAGCCGCATTACC
GTTTAGCGGGCGGTCAGCTTAACTTTATCGACAAGGCCG
ACATAGTGAAAGCCGGCGGTAAAGCGGGCCTTGGCTAT
TTATACGCCGAGCGCACCATTGACCCGAGTGATTGGTTC
TTCCAATTCCACTTCCATCAAGATCCGGTAATGCCAGG
CTCATTAGGGGTTGAAGCGATTATCGAGCTGATGCAAAC
CTATGCGATTGACCAAGACCTTGGTGCGGGCTTTAAGA
GTCCAAAATTCGGCCAGATATTATCGGATATCAAATGGA
AGTATCGCGGCCAAATCAACCCATTAAACAAACAGATG
TCGCTGGATGTGCACATTACCAGCGTGACAGACGACAAT
GGCAAACGCATCATTATGGGCGATGCCAACTTGAGTAA
AGATGGTCTGCGAATTTATGAAGTCAAAGATATCGCCAT
CTGTATTGAAGAAGCTT (SEQ ID NO: 4)
pfaD gene sequence SEQ ID NO: 5
ATGACAAGCCATACTCTCGATCAATTTAATAGTAATAAC
GAAAAACTCAGCCCTTGGCCGTGGCAAGTCAACGATGC
CGCGCTGAGCTTTGATATCGACTCATTAGGCAAAAAACT
CAAAGATTTAAGCCAAGCCTGTTACTTAGTGAATCACA
GTGAAAAAGGCTTAGGCATAGCGCAAACAGCCGAAGTAA
CCACAAGCGACAGCCAAGCGCCACTAGGCTCACACCCC
GTCAGCGCCTTTGCGCCCGCCCTTGGCACCCAAAGTTTA
GGCGACAGTAATTTTCGCCGCGTACACGGGGTTAAATA
CGCTTACTACGCTGGCGCTATGGCTAACGGTATTGCCTC
AGAAGAACTGGTTATCGCGCTGGGCCAAGCGGGCATTT
TGTGTTCGTTTGGCGCGGCGGGGTTAATCCCATCCCGCG
TTGAAGCGGCCATTACTCGCATTCAAGCGGCGCTGCCT
AATGGTCCTTACGCCTTTAATTTAATTCACAGCCCAAGC
GAGCCCGCATTAGAGCGCGGCAGTGTTGAGTTGTTCTT
AAAACATAAAGTGCGCACGGTCGAAGCCTCGGCATTTTT
AGGTTTAACGCCACAAATCGTCTATTACCGCGCAGCAG
GTTTGAGCCGCGACGCACATGGCGACATCGTCATTGGCA
ACAAAGTCATAGCCAAAATCAGTCGCACCGAAGTCGCG
ACTAAGTTTATGGAGCCGGCGCCTGCCAAAATTCTGCAG
CAATTAGTCAGTGAAGGCCTTATCAGCCAAGATCAAAT
GGCGATGGCGCAACTTGTACCCATGGCGGACGATATCAC
GGCCGAAGCCGATTCTGGCGGCCATACCGACAATCGTC
CACTGGTCACGCTATTGCCGACGATTTTGGCGCTCAAAG
ATGAAATCCAAGCTAAGTATCAATACAAGACGCCCATC
CGTGTGGGAGCAGGCGGCGGCGTTGGTACCCCCGACGCA
GCATTAGCCACCTTCAACATGGGCGCGGCCTTTATCGT
CACAGGTTCAATCAACCAAGCGTGTGTGGAGGCGGGCGC
GAGCGAACACACACGTAAGTTACTCGCCACCACAGAAA
TGGCCGATGTGACTATGGCACCCGCCGCCGATATGTTCG
AAATGGGCGTGAAATTACAAGTGGTTAAGCGTGGCACT
CTGTTCCCGATGCGCGCCAATAAGCTTTATGAAATCTAC
ACCCGTTACGATTCAATTGATGCCATCCCCGCGGACGA
GCGTAAAAAGCTCGAAGAGCAAGTGTTTCGCGCATCACT
CGATGACATTTGGGCGGGCACTGTCGCCCACTTTAACG
AGCGCGATCCTAAGCAAATCGAGCGCGCACTGGATAACC
CCAAACGTAAGATGGCGTTGATTTTCCGCTGGTATTTG
GGTCTGTCTAGCCGCTGGTCGAACACAGGTGAAGTCGGC
CGCGAAATGGATTACCAAATTTGGGCAGGCCCTGCCCT
TGGCGCATTTAATGCGTGGGCAAAAGGCAGTTATTTAGA
TGACTATAAAGCCCGTAATGCGGTCGATTTAGCCAAAC
ATTTAATGGTGGGCGCGGCCTATCAATCCCGGATTAACT
TGCTGTTATCCCAAGGGGTTAGCATTCCAGTTAGCCTG
CAACGCTGGAAACCGCTAAATCGTTTTT
(SEQ ID NO: 5)
pfaE gene sequence SEQ ID NO: 6
AATAATCAGCAAGTAAGCTCTCTATGCTGCTCAACTGCG
CGGTTAGCGTTATGGGAAGCGCGGCATTACCGGCATGC
GCTAAGCTAATGGCAAAGCGATACTCCTCGGTCAAACGA
CCAAAATGCGTTTGCCAATGCAAGGAGTCAGCTAAATC
CAGTGTGTGTATTGGGATTTGTAGCGGGACTTGTAATGG
AATATGTGGATAAACATTAAGCATTGAAGCTACGCCGC
AGGCTTGCCTCAATCCGGCATCTTGGCATGATAACTCAA
GCGATTCTGCCGCCAATTGCTCAAACGGCTTAAGCTCA
AAGGCAAAGGATTTGAGCGACTGCGCTAAACCTAAACCA
GTCGCCTTAATATAGGACTCCTTTAGCGCCCATAAATC
GAAAAAGCGCTGTCGCTGACTCGCTTCATCGGCGAGAGC
GAGCAAGGCTGCAGTTTCTTGGGGAGAAAAATAGTGAT
TTAGGATTGGATAGATATCCGTCTTCGGCCGCGACCTTT
CAATATCCACCCCAAATAAACCAGACTCAACGCCATCA
AACTGAGCCACACCAATCAATAACCAATCGCCGCTATGG
CTTAGGTTAAACTCGAGACCAGTTTGCTGCCCTAATGT
CGCCGTTAAACTCGGTTTGCCCTTAGCGCCATATTCAAA
ACACCACTCATGTGGCGATAAATCAGCATAGCGAGATA
ACACGGTCCGTAGCGCCGCCCTCACCTGTAACCCCTTTA
TTTGCACCTTGGGATCGCGATAACGCCTGACCTTTGCG
AGTTCATCATCACTTAACCAAGACTCGGCAAGTGATGTT
TGCGCCGCTGAAATATCGTTCAGTGGAATAAAAAATAG
ATCCACATTCACGCCGTTTTTGCTAGATGTTAGAGAGTC
GGTCATTCGAGAGCCTAGCTGTACAAAGCCCTGCGGCT
GATGAGTTATCATTGGCTGGCTTTTTGGAGAGCAGTTAG
CAACATTCTTATCTGGTTCATTATTGTCTAGGTTCATC
ATT (SEQ ID NO: 6)

1. A recombinant Escherichia coli Nissle 1917 (EcN) cell or a variant thereof, comprising genes encoding for pfaA, pfaB, pfaC, pfaD and pfaE, wherein the cell produces one or more omega 3 fatty acids.

2. The recombinant cell of claim 1, wherein the cell comprises a pfaA gene with at least 90% sequence identity to SEQ ID NO: 2, a pfaB gene with at least 90% sequence identity to SEQ ID NO: 3, a pfaC gene with at least 90% sequence identity to SEQ ID NO: 4, a pfaD gene with at least 90% sequence identity to SEQ ID NO: 5 and a pfaE gene with at least 90% sequence identity to SEQ ID NO: 6.

3. The recombinant cell of claim 1, wherein the cell produces eicosapentaenoic acid (EPA) and/or docosahexaenoic acid (DHA).

4. The recombinant cell of claim 3, wherein the cell produces EPA.

5. The recombinant cell of claim 1, wherein the cell is transformed with a vector comprising one or more genes selected from pfaA, pfaB, pfaC, pfaD and pfaE.

6. The recombinant cell of claim 5, wherein the one or more genes are present in the cell in a copy number greater than one.

7. The recombinant cell of claim 1, wherein the cell comprises a gene cluster with at least 90% sequence identity to SEQ ID NO: 1, wherein the gene cluster comprises pfaA, pfaB, pfaC, pfaD and pfaE.

8. The recombinant cell of claim 1, wherein the cell comprises a vector comprising a gene cluster comprising the nucleic acid sequence of SEQ ID NO:1.

9. The recombinant cell of claim 1, wherein the EcN variant comprises a genome with at least 90% sequence identity to the EcN genome deposited under GenBank under accession no. CAPM00000000.

10. (canceled)

11. The recombinant cell of claim 1, wherein the cell produces more eicosapentaenoic acid (EPA) relative to wild type S. baltica MAC1 when cultured at 15° C.

12. (canceled)

13. The recombinant cell of claim 1, wherein the cell produces at least 10 mg of eicosapentaenoic acid (EPA) per gram of cell dry weight (g⁻¹ of CDW) when cultured at 15° C.

14. A composition comprising the recombinant cell of claim 1 and a pharmaceutically acceptable carrier or a culture media.

15. The composition of claim 14, further comprising one or more prebiotics or probiotics.

16.-18. (canceled)

19. A method for producing eicosapentaenoic acid (EPA) in the gastrointestinal tract of a subject comprising orally administering to the subject the recombinant cell of claim 1.

20. (canceled)

21. The method of claim 19, wherein the subject is a human or farm animal.

22. A method for the production of omega 3 unsaturated fatty acids, comprising culturing one or more recombinant cells of claim 1 under conditions suitable for the production of omega 3 unsaturated fatty acids.

23. The method of claim 22, wherein the recombinant cell is cultured at a temperature between 5° C. and 30° C.

24. The method of claim 22, wherein the recombinant cell is cultured at a temperature between 10° C. and 25° C.

25. (canceled)

26. (canceled)

27. The method of claim 22, wherein the one or more omega 3 unsaturated fatty acids comprises eicosapentaenoic acid (EPA) and/or docosahexaenoic acid (DHA).

28. The method of claim 22, further comprising isolating the one or more omega 3 unsaturated fatty acids from the cell culture.

29.-33. (canceled)
